(19) United States
(12) Patent Application Publication
(10) Pub. No.: US 2006/0084136 A1
(43) Pub. Date: Apr. 20, 2006

(54) Production of fusion proteins by cell-free protein synthesis

(75) Inventors: Wieslaw Antoni Kudlicki, Carlsbad, CA (US); Julia Fletcher, Vista, CA (US); Federico Katzen, Carlsbad, CA (US); Robert P. Bennett, Encinitas, CA (US)

Correspondence Address: STERNE, KESSLER, GOLDSTEIN & FOX PLLC, 1100 NEW YORK AVENUE, N.W., WASHINGTON, DC, 20005, US

(73) Assignee: Invitrogen Corporation, Carlsbad, CA (US)

(21) Appl. No.: 11/181,023

(22) Filed: Jul. 14, 2005

Related U.S. Application Data
(60) Provisional application No. 60/587,583, filed on Jul. 14, 2004.

(51) Int. Cl. C12P 21/06

(52) U.S. Cl. 435/68.1

(57) ABSTRACT

The present invention relates to in vitro protein synthesis (IVPS) systems, particularly such systems using suppressor tRNAs and rare codon tRNAs to extend translation of an open reading frame into fusion protein elements, thereby generating fusion proteins in vitro. The invention also provides methods, compositions and kits using the IVPS systems of the invention, and proteins produced using the methods, compositions, kits and IVPS systems of the invention.

### CROSS REFERENCE TO RELATED APPLICATIONS

This application claims the benefit of the filing date of U.S. Provisional Application No. 60/587,583, filed Jul. 14, 2004, the disclosure of which is incorporated by reference herein in its entirety.

### BACKGROUND OF THE INVENTION

1. Field of the Invention

The present invention is in the fields of molecular biology, cell biology and protein chemistry. More specifically, the invention relates to in vitro transcription and translation systems, particularly such systems using suppressor tRNAs to site-specifically incorporate natural, unnatural or chemically modified amino acids at stop codons. The invention also provides methods for expressing peptides, polypeptides and proteins using such in vitro transcription and translations systems; compositions and kits useful in such methods; and peptides, polypeptides and proteins produced using such methods, compositions and kits.

2. Related Art

The acronym “IVTT” as used herein refers to in vitro transcription and translation. Prokaryotic cell-free systems are considered “coupled” because transcription (DNA→mRNA) and translation (mRNA→protein) occur simultaneously after the addition of DNA to the extract. However, mRNA can be used as a template for protein synthesis in E. coli extracts; in this instance, there is no requirement for transcription. Both of these systems are referred to herein as in vitro protein synthesis (IVPS) systems, but it is understood that an IVTT system is but one non-limiting type of IVPS system.

Prokaryotic IVPS systems are best exemplified by E. coli S30 cell-free extracts, which were first described by Zubay (Ann. Rev. Genet. 7:267, 1973). Commonly used eukaryotic IVPS systems include rabbit reticulocyte lysates and wheat germ extracts. Rabbit reticulocyte lysate was described by Pelham and Jackson (Eur. J. Biochem. 67:247, 1976), and wheat germ extract was described by Roberts and Paterson (Proc. Natl. Acad. Sci. USA 70:2330, 1973).

A reading frame is the nucleotide sequence of an mRNA that directly encodes a protein. A triplet codon in the mRNA encodes one amino acid in the corresponding protein. During protein synthesis, transfer RNA (tRNA) is covalently linked to (“charged with”) an amino acid. The tRNA portion of the amino acid/tRNA molecule comprises an anti-codon, which is the reverse complement of a codon and which serves to guide a charged tRNA to its correct position in a growing polypeptide chain.

A reading frame begins with an initiator codon (typically, ATG in DNA or AUG in mRNA), which encodes the first, N-terminal amino acid of a protein (typically Met or f-Met), and ends with a stop codon which does not encode an amino acid and thus serves to terminate protein synthesis. Typically, a stop codon is selected from the group consisting of: (1) TAG in DNA or UAG in mRNA; (2) TAA in DNA or UAA in mRNA; and (3) TGA in DNA or UGA in mRNA. See Stryer, L., Biochemistry, 3^{rd} Ed., New York: W.H. Freeman and Co., pp. 104-108 (1988).

However, in vivo genetic studies have identified mutations that result in a tRNA molecule that has an anti-codon sequence that is the reverse complement of a stop codon. These tRNAs thus lead to the incorporation of an amino acid at the C-terminal position of a protein where, normally, the stop codon would end the protein. If there are no other obstacles to translation, protein synthesis may continue and add more amino acids to the C-terminal protein. Such mutant tRNAs were first identified by mutations that add stop codons that were introduced into genes of interest; the mutant tRNA allowed the full-length protein to be expressed, thereby “suppressing” the effect of the loss of the full-length protein. Accordingly, they are known as suppressor mutations. Certain suppressor mutations, termed “amber mutations,” suppress TAG/UAG stop codons. Other suppressor mutations, termed “ochre mutations,” suppress TAA/UAA stop codons. Still other suppressor mutations, termed “opal mutations,” suppress TGA/UGA stop codons. See Darnell, J., et al., Molecular Cell Biology, New York: Scientific American Books, Inc., pp. 121-124 (1986).

### BRIEF SUMMARY OF THE INVENTION

The present invention is generally directed to systems for in vitro transcription and translation, and compositions and methods therefor.

In certain aspects, the invention provides an in vitro protein synthesis (IVPS) composition that includes an extract of a cell or organism supplemented with one or more exogenously added tRNAs. In this and other embodiments, the tRNA genes produce tRNA molecules selected from the group consisting of rare codon tRNA molecules, suppressor tRNA molecules, mutant tRNA molecules and non-endogenous tRNA molecules.

In one aspect of the invention, supplemented tRNAs are suppressor tRNAs. In this case, the added suppressor tRNAs promote readthrough of one or more stop codons in a nucleic acid template used for protein synthesis. In some embodiments, the suppressor tRNAs can be used to incorporate modified or nonnaturally-occurring amino acids into a protein.

In other preferred embodiments, one or more tRNAs added to an in vitro protein synthesis system allows readthrough from one open reading frame (ORF) into a second open reading frame of a nucleic acid template, where the two open reading frames are linked in a nucleic acid construct by a sequence encoding a stop codon. Thus, use of the translation system permits the production of a protein of interest in unfused and fusion protein form without the need for generating separate constructs. A protein of interest in unfused form and as a fusion protein with, for example, a reporter protein or peptide, can be synthesized in vitro, in parallel if desired (see FIG. 3 for example), from the same construct.

The ability of a translation system having added suppressor tRNA to allow readthrough of a stop codon can be greatly enhanced by the addition of a release factor (RF) inhibitor to the in vitro translation reaction. The inventors have found that suppression of translation termination at a stop codon can be so efficient using added tRNA and an RF antibody that fusion proteins resulting from stop codon suppression are the majority of synthesized protein from such reactions. Thus, the invention includes in vitro protein synthesis systems and methods in which both suppressor tRNAs and an inhibitor of an RF are added to a cell extract for stop codon suppression during in vitro translation.

In addition, the invention includes IVPS systems in which translation of proteins is enhanced by the addition of tRNAs that recognize “rare” codons. In this aspect, the invention provides methods for increasing protein production in cases where the gene of interest includes one or more codons that are infrequently used in the cell or organism from which the translation extract was obtained. In this case supplementing the in vitro translation system with tRNAs that recognize such rare codons can greatly increase the yield of protein synthesis.

The invention also provides methods of synthesizing proteins in vitro, comprising contacting a nucleic acid molecule (e.g., a DNA molecule or an RNA molecule, and preferably a messenger RNA (“mRNA”) molecule) with at least one of the compositions of the present invention, under conditions favoring the transcription and/or translation of the nucleic acid molecule, thereby synthesizing a protein, peptide or polypeptide that is encoded by the nucleic acid molecule. Additional such methods of the invention further comprise separating or isolating the protein (or peptide or polypeptide) from the compositions of the invention following in vitro transcription and/or translation of the nucleic acid molecule. In additional embodiments, the invention also provides proteins, peptides and polypeptides synthesized by such methods of the invention. In related embodiments, the invention also provides arrays comprising one or more, two or more, three or more, etc., of the proteins, peptides or polypeptides synthesized by the methods of the present invention, wherein the proteins, peptides or polypeptides are immobilized into an ordered array on discrete sites on a solid support, such as a glass slide, a microchip, a microtiter plate, a chromatography support, a nanotube, and the like.

The invention also provides kits comprising the in vitro protein synthesis compositions of the present invention. Certain such kits, or alternative embodiments, may comprise solid supports such as microtiter plates comprising, or at least partially coated by, the in vitro protein synthesis compositions of the invention. In other embodiments, the invention provides instruments, such as electronic detection instruments like plate readers, comprising the microtiter plates or arrays of the invention.

### BRIEF DESCRIPTION OF THE DRAWINGS

These and other objects and advantages of the present invention will be apparent upon consideration of the following detailed description, taken in conjunction with the accompanying drawings, in which:

FIG. 1. Scheme in which a protein is labeled by suppression, which results in the incorporation of a detectably labeled amino acid at a codon that would normally function as a stop codon.

FIG. 2. One version of a scheme of the invention, wherein suppression of a stop codon using a natural (untagged) amino acid results in read-through translation, thereby creating a fusion protein comprising the protein of interest and one or more fusion protein elements. Three such elements are shown (label, protease cleavage site, affinity tag).

FIG. 3. Scheme for parallel preparation of wildtype (untagged) and fusion (tagged) forms of a protein of interest starting from one culture of cells.

FIG. 4. Vector maps of pACYCtRNA3 (FIG. 4A; 3 tRNA genes), and pACYCtRNA6 (FIG. 4B; 6 tRNA genes).

FIG. 5. Q sepharose column analysis of tRNA. FIG. 5A: Chromatograph of elution peaks from 10ml Q-sepharose column. FIG. 5B: 10% TBE-Urea gel of column fractions run across column peaks. Lanes 1-10 and 13 in the gel depicted in FIG. 5B correspond to peak E12-15 & F1-F7, respectively, on the chromatograph shown in FIG. 5A; Lanes 14-21 correspond to peaks F8-F15; Lanes 11 and 22 are Roche tRNA; Lanes 12 and 23 are 10 bp DNA ladder. FIG. 5C: 10% TBE-Urea gel of purified tRNA compared to Roche tRNA. Lane 1, BL21 cells before induction; Lane 2, BL21 cells after induction with IPTG; Lane 3, 300 ng Roche tRNA; Lane 4, 3 mg Roche tRNA; Lane 5, 300 ng Rare tRNA; Lane 6, 3mg Rare tRNA.

FIG. 6. Comparison of the addition of Roche and BL21 Star™ tRNAs in Expressway™ Linear reactions. FIG. 6A: An autoradiograph of a 4-20% tris/glycine gradient gel of b-gal from 1 ml from 50 ml Expressway™ Linear reactions. Lane 1, 8mg Roche tRNA; Lane 2, 12 μg Roche tRNA; Lane 3, 16 μg Roche tRNA, Lane 4, 8 μg BL21 tRNA; Lane 5, 12 mg of BL21 tRNA; Lane 6, 16 μg of BL21 tRNA; Lane 7, no tRNA; Lane 8, no DNA. FIG. 6B: A graph of b-gal expression from reactions depicted and described in FIG. 6A. FIG. 6C: A graph of expression from linear GFP in 50 ml Expressway™ Linear reactions after the addition of 14 μg each of Roche and BL21 tRNA.

FIG. 7. Comparison of the addition of Roche and Rare tRNAs in Expressway™ Plus reactions with Sso SSB expression. FIG. 7A: An autoradiograph of a 4-12% bis/tris gradient gel of Sso SSB expression from 1 ml from 50 μl Expressway™ Plus reactions. Lane 1, no tRNA; Lane 2, Roche tRNA; Lane 3, RarepACYCtRNA6; Lane 4, no DNA. FIG. 7B: The coomassie stain of the gel depicted in FIG. 7A. FIG. 7C: A graph of Sso SSB expression from the reactions above.

FIG. 8. Diagrams of the pEXP4-DEST vector (FIG. 8A) and the pEXP4/ORF-TAG expression vector (FIG. 8B).

FIG. 9. Purification of IVT (in vitro transcribed) stRNA. FIG. 9A:

Chromatograph of column fractions from a Q sepharose column loaded with IVT stRNA. FIG. 9B: 10% TBE-urea gel of column fractions from the chromatograph in FIG. 9A. FIG. 9C: 10% TBE-urea gel of different preparations of stRNA (Lane 1, FPLC IVT stRNA; Lane 2 GelPur. IVT stRNA; Lane 3, LiCl2 IVT stRNA; Lane 4, Total stRNA; L=100 bp DNA ladder.

FIG. 10. Titration of suppressor tRNA. FIG. 10A: In-gel detection of Lumio™-tagged similar to creatine kinase (SCK) protein on a 4-12% NuPage Bis/Tris gel from reactions containing titrated stRNA and SCK in pEXP4 (Lanes 2-7, FPLC IVT stRNA; Lanes 9-14 Gel Pur. IVT stRNA; Lanes 17-22, Total stRNA; Lanes 1, 8, 16 no stRNA; Lanes 15, 23 no DNA: L=Benchmark™ Fluorescent markers). Molecular weights are indicated to the side of the gels. FIG. 10B: Phosphorimage of gel in FIG. 10A, tagged and untagged proteins are indicated. FIG. 10C: Graph of SCK protein yield from stRNA titration reactions shown in FIGS. 10A and 10B as determined by ³⁵S-methionine incorporation. FIG. 10D: A table of read-through percentages from the stRNA titration reactions shown in FIGS. 10A and 10B as determined by phosphorimage analysis.

FIG. 11. Titration of purified RF1 antibody. FIG. 11A: In-gel detection of Lumio™-tagged creatine kinase B (CKB) protein on a 4-12% NuPage Bis/Tris gel from reactions containing titrated RF1 antibody, Total stRNA (10 μg) and CKB in pEXP4 (Lane 4=no stRNA; Lane 5=no DNA. FIG. 11B: Phosphorimage of gel in FIG. 10A, tagged and untagged proteins are indicated (L=Benchmark™ fluorescent marker). FIG. 11C: Graph of CKB protein yield from reactions shown in FIGS. 11A and 11B as determined by ³⁵S-methionine incorporation (gel lane numbers are indicated on graph bars). FIG. 11D: Table of percent read-through from reactions shown in FIGS. 11A and 11B as determined by phosphorimage analysis.

FIG. 12. Representative Lumio™ Detection and Autoradiograph of pEXP4-Human ORF Clones: Gel Pur. IVT stRNA and Purified RF1 Antibody Addition. FIG. 12A: In-gel detection of Lumio™-tagged proteins of pEXP4-Human ORF clones with the addition of IVT stRNA (10 g) and RF1 AB (8 μg) (CKB=Creatine Kinase Brain; cDPK=cAMP Dependent Protein Kinase; SCK=Similar to Creatine Kinase; CKE1=Casein Kinase Epsilon 1; STPK=Serine/Threonine Protein Kinase). FIG. 12B: Phosphorimage of gel in FIG. 12A (L=Benchmark fluorescent marker).

FIG. 13. Real-time detection of pEXP4-Human ORF clones with and without the addition of IVT stRNA and RF1 Antibody. FIG. 13A: Plot of relative fluorescence from a pEXP4-SCK clones in reactions with and without the addition of IVT stRNA (10 μg). FIG. 13B: Plot of relative fluorescence from a pEXP4-SCK clones in reactions with and without the addition of IVT stRNA and RF1 antibody (8 μg).

FIG. 14. Comparison of % read-through: Freeze/Thaw and Addition of RF1 Antibody. FIG. 14A: In-gel detection of Lumio™-tagged cAMP Dependent Protein Kinase run on a 4-12% NuPage Bis/Tris gel.

FIG. 14B: Phosphorimage of reactions shown in FIG. 14A (L, Benchmark fluorescent marker). FIG. 14C: Table of % read-through from reactions shown in FIG. 14B as determined by phosphorimage analysis.

FIG. 15. Western blot analysis of pEXP4 human ORF clones after in-gel detection with Lumio™. FIG. 15A: In-gel detection of Lumio™-tagged pEXP4 ORFs on a 4-12% NuPage Bis/Tris gel in reactions with and without suptRNA. Lanes 1 and 2, creatine kinase B; Lanes 3 and 4, cAMP dependent protein kinase; Lanes 5 and 6, similar to creatine kinase; Lanes 7 and 8, casein kinase epsilon 1; Lanes 9 and 10, serine/threonine protein kinase; Lane 11 no DNA. FIG. 15B: Western blot of gel shown in FIG. 1 5A probed with an Anti-His (C-terminal) HRP-linked antibody (Invitrogen) and developed with ECL (Amersham).

### DETAILED DESCRIPTION OF THE INVENTION

I. Definitions

In the description that follows, a number of terms used in recombinant nucleic acid technology are utilized extensively. In order to provide a clear and more consistent understanding of the specification and claims, including the scope to be given such terms, the following definitions are provided.

Amino Acids: As used herein, the following is the set of 20 naturally occurring amino acids commonly found in proteins and the one and three letter codes associated with each amino acid:

TABLE 1

Naturally Occurring Amino Acids
and the Genetic Code

| | 3-Letter | 1-Letter | |
|---|---|---|---|
| Full name | Code | Code | Standard Codons¹ |
| Alanine | Ala | A | GCU, GCC, GCA, |
| | | | GCG |
| Arginine | Arg | R | CGU, CGC, CGA, |
| | | | CGG, AGA, AGG |
| Asparagine | Asn | N | AAU, AAC |
| Aspartic Acid | Asp | D | GAU, GAC |
| Cysteine | Cys | C | UGU, UGC |
| Glutamine | Gln | Q | CAA, CAG |
| Glutamic Acid | Glu | E | GAA, GAG |
| Glycine | Gly | G | CGU, CGC, CGA, |
| | | | CGG |
| Histidine | His | H | CAU, CAC |
| Isoleucine | Ile | I | AUU, AUC, AUA |
| Leucine | Leu | L | UUA, UUG, CUU, |
| | | | CUC, CUA, CUG |
| Lysine | Lys | K | AAA, AAG |
| Methionine | Met | M | AUG |
| Phenylalanine | Phe | F | UUU, UUC |
| Proline | Pro | P | CCU, CCC, CCA, |
| | | | CCG |
| Serine | Ser | S | UCU, UCC, UCA, |
| | | | UCG, AGU, AGC |
| Threonine | Thr | T | ACU, ACC, ACA, |
| | | | ACG |
| Tryptophan | Trp | W | UGG |
| Tyrosine | Tyr | Y | UAU, UAC |
| Valine | Val | V | GUU, GUC, GUA, |
| | | | GUG |

¹Codons are depicted in this table as they appear in mRNA. Corresponding codons in DNA molecules would substitute a thymidine (T) nucleotide for any uracil (U) nucleotide in the RNA sequence.

Gene: As used herein, the term “gene” refers to a nucleic acid that contains information necessary for expression of a polypeptide, protein, or untranslated RNA (e.g., rRNA, tRNA, anti-sense RNA). When the gene encodes a protein, it includes the promoter and the structural gene open reading frame sequence (ORF), as well as other sequences involved in expression of the protein. When the gene encodes an untranslated RNA, it includes the promoter and the nucleic acid that encodes the untranslated RNA.

Structural Gene: As used herein, the phrase “structural gene” refers to a nucleic acid that is transcribed into messenger RNA that is then translated into a sequence of amino acids characteristic of a specific polypeptide.

IVT: The terms “in vitro transcription” (IVT) and “cell-free transcription” are used interchangeably herein and are intended to refer to any method for cell-free synthesis of RNA from DNA without synthesis of protein from the RNA. A preferred RNA is messenger RNA (mRNA), which encodes proteins.

IVTT: The terms “in vitro transcription-translation” (IVTT), “cell-free transcription-translation”, “DNA template-driven in vitro protein synthesis” and “DNA template-driven cell-free protein synthesis” are used interchangeably herein and are intended to refer to any method for cell-free synthesis of mRNA from DNA (transcription) and of protein from mRNA (translation).

IVPS: The terms “in vitro protein synthesis” (IVPS), “in vitro translation”, “cell-free translation”, and “cell-free protein synthesis” are used interchangeably herein and are intended to refer to any method for cell-free synthesis of a protein. IVPS may or may not include transcription in the same reaction as translation. “RNA template-driven in vitro protein synthesis”, “RNA template-driven cell-free protein synthesis” are included as non-limitng examples of IVPS, as is IVTT.

Detectably labeled: The terms “detectably labeled” and “labeled” are used interchangeably herein and are intended to refer to situations in which a molecule (e.g., a nucleic acid molecule, protein, nucleotide, amino acid, and the like) have been tagged with another moiety or molecule that produces a signal capable of being detected by any number of detection means, such as by instrumentation, eye, photography, radiography, and the like. In such situations, molecules can be tagged (or “labeled”) with the molecule or moiety producing the signal (the “label” or “detectable label”) by any number of art-known methods, including covalent or ionic coupling, aggregation, affinity coupling (including, e.g., using primary and/or secondary antibodies, either or both of which may comprise a detectable label), and the like. Suitable detectable labels for use in preparing labeled or detectably labeled molecules in accordance with the invention include, for example, radioactive isotope labels, fluorescent labels, chemiluminescent labels, bioluminescent labels and enzyme labels, and others that will be familiar to those of ordinary skill in the art.

Host: As used herein, the term “host” refers to any prokaryotic or eukaryotic (e.g., mammalian, insect, yeast, plant, avian, animal, etc.) organism that is a recipient of a replicable expression vector, cloning vector or any nucleic acid molecule. The nucleic acid molecule may contain, but is not limited to, a sequence of interest, a transcriptional regulatory sequence (such as a promoter, enhancer, repressor, and the like) and/or an origin of replication. As used herein, the terms “host,” “host cell,” “recombinant host” and “recombinant host cell” may be used interchangeably. For examples of such hosts, see Sambrook, et al., Molecular Cloning: A Laboratory Manual, Cold Spring Harbor Laboratory, Cold Spring Harbor, N.Y.

Transcriptional Regulatory Sequence: As used herein, the phrase “transcriptional regulatory sequence” refers to a functional stretch of nucleotides contained on a nucleic acid molecule, in any configuration or geometry, that act to regulate the transcription of (1) one or more structural genes (e.g., two, three, four, five, seven, ten, etc.) into messenger RNA or (2) one or more genes into untranslated RNA. Examples of transcriptional regulatory sequences include, but are not limited to, promoters, enhancers, repressors, operators (e.g., the tet operator), and the like.

Promoter: As used herein, a promoter is an example of a transcriptional regulatory sequence, and is specifically a nucleic acid generally described as the 5′-region of a gene located proximal to the start codon or nucleic acid that encodes untranslated RNA. The transcription of an adjacent nucleic acid segment is initiated at or near the promoter. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions.

Repression Cassette: As used herein, the phrase “repression cassette” refers to a nucleic acid segment that contains a repressor or a selectable marker present in the subcloning vector.

Primer: As used herein, the term “primer” refers to a single stranded or double stranded oligonucleotide that is extended by covalent bonding of nucleotide monomers during amplification or polymerization of a nucleic acid molecule (e.g., a DNA molecule). In one aspect, the primer may be a sequencing primer (for example, a universal sequencing primer). In another aspect, the primer may comprise a recombination site or portion thereof.

Template: As used herein, the term “template” refers to a double stranded or single stranded nucleic acid molecule that is to be amplified, synthesized or sequenced. In the case of a double-stranded DNA molecule, denaturation of its strands to form a first and a second strand may be performed before these molecules may be amplified, synthesized or sequenced, or the double stranded molecule may be used directly as a template. For single stranded templates, a primer complementary to at least a portion of the template hybridizes under appropriate conditions and one or more polypeptides having polymerase activity (e.g., two, three, four, five, or seven DNA polymerases and/or reverse transcriptases) may then synthesize a molecule complementary to all or a portion of the template. Alternatively, for double stranded templates, one or more transcriptional regulatory sequences (e.g., two, three, four, five, seven or more promoters) may be used in combination with one or more polymerases to make nucleic acid molecules complementary to all or a portion of the template. The newly synthesized molecule, according to the invention, may be of equal or shorter length compared to the original template. Mismatch incorporation or strand slippage during the synthesis or extension of the newly synthesized molecule may result in one or a number of mismatched base pairs. Thus, the synthesized molecule need not be exactly complementary to the template. Additionally, a population of nucleic acid templates may be used during synthesis or amplification to produce a population of nucleic acid molecules typically representative of the original template population.

Incorporating: As used herein, the term “incorporating” means becoming a part of a nucleic acid (e.g., DNA) molecule or primer.

Library: As used herein, the term “library” refers to a collection of nucleic acid molecules (circular or linear). In one embodiment, a library may comprise a plurality of nucleic acid molecules (e.g., two, three, four, five, seven, ten, twelve, fifteen, twenty, thirty, fifty, one hundred, two hundred, five hundred one thousand, five thousand, or more), that may or may not be from a common source organism, organ, tissue, or cell. In another embodiment, a library is representative of all or a portion or a significant portion of the nucleic acid content of an organism (a “genomic” library), or a set of nucleic acid molecules representative of all or a portion or a significant portion of the expressed nucleic acid molecules (a cDNA library or segments derived there from) in a cell, tissue, organ or organism. A library may also comprise nucleic acid molecules having random sequences made by de novo synthesis, mutagenesis of one or more nucleic acid molecules, and the like. Such libraries may or may not be contained in one or more vectors (e.g., two, three, four, five, seven, ten, twelve, fifteen, twenty, thirty, fifty, etc.).

Amplification: As used herein, the term “amplification” refers to any in vitro method for increasing the number of copies of a nucleic acid molecule with the use of one or more polypeptides having polymerase activity (e.g., one, two, three, four or more nucleic acid polymerases or reverse transcriptases). Nucleic acid amplification results in the incorporation of nucleotides into a DNA and/or RNA molecule or primer thereby forming a new nucleic acid molecule complementary to a template. The formed nucleic acid molecule and its template can be used as templates to synthesize additional nucleic acid molecules. As used herein, one amplification reaction may consist of many rounds of nucleic acid replication. DNA amplification reactions include, for example, the polymerase chain reaction (PCR). One PCR reaction may consist of 5 to 100 cycles of denaturation and synthesis of a DNA molecule.

Nucleotide: As used herein, the term “nucleotide” refers to a base-sugar-phosphate combination. Nucleotides are monomeric units of a nucleic acid molecule (DNA and RNA). The term nucleotide includes ribonucleoside triphosphates ATP, UTP, CTG, GTP and deoxyribonucleoside triphosphates such as dATP, dCTP, dITP, dUTP, dGTP, dTTP, or derivatives thereof. Such derivatives include, for example, [α-S]dATP, 7-deaza-dGTP and 7-deaza-dATP. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP. In describing certain aspects of the present invention, the present specification may express a nucleotide or nucleotide sequence (e.g., a trinucleotide sequence such as a codon) in the form of only a DNA sequence or an RNA sequence. However, the present invention contemplates, and one of ordinary skill would readily understand, that the expression of a nucleotide or nucleotide sequence only in terms of a DNA sequence or an RNA sequence is intended to refer to the corresponding RNA sequence or DNA sequence, as the case may be. For example, a codon may be expressed only as “TGA” herein, i.e., by the DNA sequence for that codon. It is intended, however, that this expression also refers to the corresponding RNA sequence in which a uracil base (“U”) is substituted for the thymine base (“T”) in the DNA sequence, such that the corresponding RNA sequence is “UGA.” According to the present invention, a “nucleotide” may be unlabeled or detectably labeled by well known techniques. Detectable labels include, for example, radioactive isotopes, fluorescent labels, chemiluminescent labels, bioluminescent labels and enzyme labels.

Nucleic Acid Molecule: As used herein, the phrase “nucleic acid molecule” refers to a sequence of contiguous nucleotides (riboNTPs, dNTPs, ddNTPs, or combinations thereof) of any length. A nucleic acid molecule may encode a full-length polypeptide or a fragment of any length thereof, or may be non-coding. As used herein, the terms “nucleic acid molecule” and “polynucleotide” may be used interchangeably and include both RNA and DNA.

Oligonucleotide: As used herein, the term “oligonucleotide” refers to a synthetic or natural molecule comprising a covalently linked sequence of nucleotides that are joined by a phosphodiester bond between the 3′ position of the pentose of one nucleotide and the 5′ position of the pentose of the adjacent nucleotide.

Polypeptide: As used herein, the term “polypeptide” refers to a sequence of contiguous amino acids of any length. The terms “peptide,” “oligopeptide,” or “protein” may be used interchangeably herein with the term “polypeptide.”

Other terms used in the fields of recombinant nucleic acid technology and molecular and cell biology as used herein will be generally understood by one of ordinary skill in the applicable arts.

II. FlAsH™, ReAsH and LUMIO™

LUMIO™ is the tradename for reagents, FlAsH™ and ReAsH (Invitrogen Corporation; Carlsbad, Calif.), that bind to and label recombinant and fusion proteins of interest. Binding of FlAsH™ (also called LUMIO™ Green) to its target sequence causes the ligand to emit a strong green fluorescence, whereas binding of ReAsH leads to red fluorescence.

II.A. FlAsH™

The Fluorescein Arsenical Hairpin binding (FlAsH™) labeling reagent, EDT₂[4′,5′-bis(1,3,2-dithioarsolan-2-yl)fluorescein-(1,2-ethanedithiol)₂], is a bisarsenical compound that binds to polypeptides comprising the sequence, C—C—X—X—C—C (SEQ ID NO:7), wherein “C” represents cysteine and “X” represents any amino acid other than cysteine (Griffin et al. Science 281:269-272, 1998). Adams et al. (Am Chem Soc. 124:6063-6076, 2002) have reported that the highest affinity is achieved when X—X is proline and glycine. FlAsH tags have been successfully incorporated at either the N- or C-termini of proteins, as well as exposed surface regions within a protein (Griffin et al., 1998; Adams et al., 2002; and Griffin et al. Methods Enzymol. 327:565-78, 2000).

The bisarsenical dye is normally reacted with two ethylenedithiol (EDT) molecules for easier diffusion through the cell membrane. The FLASH™-EDT₂ labeling reagent is non-fluorescent and becomes fluorescent upon binding to the “FLASH-tag” tetracysteine motif. When the FlAsH-EDT₂ dye is not bound to a protein, the small size of the EDT permits the free rotation of the arsenium atoms that quench the fluorescence of the fluorescein moiety. When a C—C—P-G-C—C labeled protein is mixed with the FlAsH-EDT₂ dye, the arsenium atoms of the FlAsH™ dye react with the tetracysteine tag of the protein and form covalent bonds. The product of this reaction does not allow free rotation of the arsenium atoms and, because they no longer quench its fluorescence, the fluorescein moiety becomes fluorescent. The increase of the fluorescence is about 50,000 fold when the FlAsH dye is bound to protein (Griffin et al., 1988).

This quenching of the fluorescence of the FlAsH dye when not bound and recovering the full fluorescence when bound makes it highly suitable for detection of proteins. Although the FlAsH dye can react with other cysteines in the protein molecule that are not part of the FlAsH tag, the affinity for the other cysteines is significantly lower. Therefore, a small amount of a protein containing the FlAsH tag can be detected in the presence of large quantities of other proteins.

The FlAsH-EDT₂ reagent is also useful for in cell assays because this reagent can freely diffuse across the cell membranes of live mammalian cells and bind to proteins engineered to contain the FlAsH-tag. This allows for in vivo detection and subcellular localization of specific proteins without the need for time-consuming immunostaining (Griffin et al., 1998; Adams et al., 2002; and Griffin et al., 2000).

In addition to labeling of specific proteins in live cells, the FlAsH-EDT₂ reagent can also be used to detect FLASH-tagged proteins in SDS-PAGE gels (Adams et al., 2002). Inclusion of the FlAsH-EDT₂ reagent in the sample loading buffer allows rapid detection of recombinant proteins in whole cell lysates using a standard ultraviolet (UV) lightbox, without the need for western blotting or other more laborious protein detection methods.

The FlAsH-EDT₂ reagent can also in affinity purification of proteins comprising the C—C—X—X—C—C sequence. Thorn et al. (A novel method of affinity-purifying proteins using a bis-arsenical fluorescein. Protein Sci. 9:213, 2000) report that kinesin tagged with this sequence binds specifically to FlAsH resin and can be eluted in a fully active form. Thorn et al. reported that the protein obtained with a single FlAsH chromatographic step from crude Escherichia coli lysates is purer than that obtained with nickel affinity chromatography of 6×His tagged kinesin. Further, protein bound to the FlAsH column can be completely eluted by dithiothreitol, which is unlike nickel affinity chromatography, which requires high concentrations of imidazole or pH changes for elution.

II.B. ReAsH

ReAsH is a variant of FlAsH that is useful for electron microscopy (EM), because it can generate singlet oxygen upon illumination. Singlet oxygen drives localized polymerization of the substrate diaminobenzidene (DAB) into an insoluble form that can be viewed by EM. Because the fluorescent label binds directly to the protein of interest, and the DAB polymer deposits directly nearby the fluorophore, the resolution is better than traditional methods, such as immunogold labeling. Additionally, this technique does not require the diffusion of large antibodies into the fixed specimens. See, for example, Daniel and Postma, Molecular Interventions 2:132, 2002; Gaietta et al., Multicolor and electron microscopic imaging of connexin trafficking. Science 296:503, 2002.

II.C. Patent Documents Relating to FlAsH

U.S. Pat. No. 5,932,474 to Tsien et al., entitled “Target Sequences for Synthetic Molecules”.

U.S. Pat. No. 6,054,271 to Tsien et al., entitled “Methods of Using Synthetic Molecules and Target Sequences”.

U.S. Pat. Nos. 6,451,569 and 6,008,378, published U.S. patent application No. 2003/0083373, and published PCT Patent Application WO 99/21013, all to Tsien et al. and all entitled “Synthetic Molecules that Specifically React with Target Sequences”.

III. Transfer RNA and Suppression

In the invention, transfer RNA (tRNA) genes and tRNA molecules are manipulated to cause suppression of a stop codon or, additionally or alternatively, to enhance the production of a protein encoded by a cloned gene having a codon bias from one organism and being expressed in an expression system.

III.A. Suppressor tRNAs

Mutant tRNA molecules that recognize what are ordinarily stop codons suppress the termination of translation of an mRNA molecule and are termed suppressor tRNAs. Three codons are used by both eukaryotes and prokaryotes to signal the end of gene. When transcribed into mRNA, the codons have the following sequences: UAG (amber), UGA (opal) and UAA (ochre). Under most circumstances, the cell does not contain any tRNA molecules that recognize these codons. Thus, when a ribosome translating an mRNA reaches one of these codons, the ribosome stalls and falls of the RNA, terminating translation of the mRNA. The release of the ribosome from the mRNA is mediated by specific factors (see S. Mottagui-Tabar, Nucleic Acids Research 26(11), 2789, 1998). A gene with an in-frame stop codon (TAA, TAG, or TGA) will ordinarily encode a protein with a native carboxy terminus. However, suppressor tRNAs can result in the insertion of amino acids and continuation of translation past stop codons.

A number of such suppressor tRNAs have been found. Examples include, but are not limited to, the supE, supP, supD, supF and supZ suppressors, which suppress the termination of translation of the amber stop codon, supB, gIT, supL, supN, supC and supM suppressors, which suppress the function of the ochre stop codon and glyT, trpT and Su-9 suppressors, which suppress the function of the opal stop codon. In general, suppressor tRNAs contain one or more mutations in the anti-codon loop of the tRNA that allows the tRNA to base pair with a codon that ordinarily functions as a stop codon. The mutant tRNA is charged with its cognate amino acid residue and the cognate amino acid residue is inserted into the translating polypeptide when the stop codon is encountered. For a more detailed discussion of suppressor tRNAs, the reader may consult Eggertsson, et al., (1988) Microbiological Review 52(3):354-374, and Engleerg-Kukla, et al. (1996) in Escherichia coli and Salmonella Cellular and Molecular Biology, Chapter 60, pps 909-921, Neidhardt, et al. eds., ASM Press, Washington, D.C.

Mutations that enhance the efficiency of termination suppressors, i.e., increase the read-through of the stop codon, have been identified. These include, but are not limited to, mutations in the uar gene (also known as the prfA gene), mutations in the ups gene, mutations in the sueA, sueB and sueC genes, mutations in the rpsD (ramA) and rpsE (spcA) genes and mutations in the rplL gene.

Under ordinary circumstances, host cells would not be expected to be healthy if suppression of stop codons is too efficient. This is because of the thousands or tens of thousands of genes in a genome, a significant fraction will naturally have one of the three stop codons; complete read-through of these would result in a large number of aberrant proteins containing additional amino acids at their carboxy termini. If some level of suppressing tRNA is present, there is a race between the incorporation of the amino acid and the release of the ribosome. Higher levels of tRNA may lead to more read-through although other factors, such as the codon context, can influence the efficiency of suppression.

Organisms ordinarily have multiple genes for tRNAs. Combined with the redundancy of the genetic code (multiple codons for many of the amino acids), mutation of one tRNA gene to a suppressor tRNA status does not lead to high levels of suppression. The TAA/UAA stop codon is the strongest, and most difficult to suppress. The TGA/UGA is the weakest, and naturally (in E. coli) leaks to the extent of 3%. The TAG/UAG (amber) codon is relatively tight, with a read-through of ˜1% without suppression. In addition, the amber codon can be suppressed with efficiencies on the order of 50% with naturally occurring suppressor mutants. Suppression in some organisms (e.g., E. coli) may be enhanced when the nucleotide following the stop codon is an adenosine. Thus, the present invention contemplates nucleic acid molecules having a stop codon followed by an adenosine (e.g., having the sequence TAGA, TAAA, and/or TGAA).

Suppression has been studied for decades in bacteria and bacteriophages. In addition, suppression is known in yeast, flies, plants and other eukaryotic cells including mammalian cells. For example, Capone, et al. (Molecular and Cellular Biology 6(9):3059-3067, 1986) demonstrated that suppressor tRNAs derived from mammalian tRNAs could be used to suppress a stop codon in mammalian cells. A copy of the E. coli chloramphenicol acetyltransferase (cat) gene having a stop codon in place of the codon for serine 27 was transfected into mammalian cells along with a gene encoding a human serine tRNA that had been mutated to form an amber, ochre, or opal suppressor derivative of the gene. Successful expression of the cat gene was observed. An inducible mammalian amber suppressor has been used to suppress a mutation in the replicase gene of polio virus and cell lines expressing the suppressor were successfully used to propagate the mutated virus (Sedivy, et al., Cell 50: 379-389 (1987)). The context effects on the efficiency of suppression of stop codons by suppressor tRNAs has been shown to be different in mammalian cells as compared to E. coli (Phillips-Jones, et al., Molecular and Cellular Biology 15(12): 6593-6600 (1995), Martin, et al., Biochemical Society Transactions 21: (1993)) Since some human diseases are caused by nonsense mutations in essential genes, the potential of suppression for gene therapy has long been recognized (see Temple, et al., Nature 296(5857):537-40 (1982)). The suppression of single and double nonsense mutations introduced into the diphtheria toxin A-gene has been used as the basis of a binary system for toxin gene therapy (Robinson, et al., Human Gene Therapy 6:137-143 (1995)).

III.B. Mutant and Variant tRNA Molecules

In some embodiments of the invention, suppression is achieved through the use of a tRNA/codon pairing in which the tRNA recognizes codons having 4, 5, 6 or more nucleotide bases, as opposed to the natural triplet codon. See, for example, Anderson et al., An expanded genetic code with a functional quadruplet codon. Proc Natl Acad Sci USA 101:7566 (Epub 2004 May 11); O'Connor Insertions in the anticodon loop of tRNA1Gln(sufG) and tRNA(Lys) promote quadruplet decoding of CAAA. Nucleic Acids Res. 30:1985, 2002; Moore et al., Decoding of tandem quadruplets by adjacent tRNAs with eight-base anticodon loops. Nucleic Acids Res. 28:3615, 2000; Moore et al., Quadruplet codons: implications for code expansion and the specification of translation step size. J Mol Biol. 298:195, 2000; Bossi et al., Four-base codons ACCA, ACCU and ACCC are recognized by frameshift suppressor sufj. Cell 25:489, 1981.

Suppression can also be achieved using tRNA from organelles, such as mitochondria and chloroplasts, that have a non-standard genetic code. For example, in mammalian, echinoderm and yeast mitochondria, UGA=Trp, rather than stop); in the mitochondria of Ciliate, Dasycladacean and Hexamita, UAA and UAG both=Gln, rather than stop; in Euplotid mitochondria, UGA=Cys; and in flatworm mitochondria, UAA=Tyr and UGA=Trp.

See Keeling and Doolittle. A non-canonical genetic code in an early diverging eukaryotic lineage. EMBO J. 15:2285, 1996; Yokobori et al.

Genetic code variations in mitochondria: tRNA as a major determinant of genetic code plasticity. J Mol Evol. 53:314, 2001; and www.no.embnet.org/Resources/Data/allcodes.php3#SG2.

III.C. Use of Suppressor tRNAs to Conditionally Express Fusion Proteins

Because the methods used to create the nucleic acids of the invention are site specific, the orientation and/or reading frame of a nucleic acid sequence on a first nucleic acid molecule can be controlled with respect to the orientation and/or reading frame of a sequence on a second nucleic acid molecule when all or a portion of the molecules are joined in a recombination and/or topoisomerase-mediated reaction. This control makes the construction of fusions between sequences present on different nucleic acid molecules a simple matter.

In general terms, an open reading frame may be expressed in four forms: native at both amino and carboxy termini, modified at either end, or modified at both ends. The portion of a nucleic acid sequence encoding a polypeptide of interest may be referred to as an open reading frame (ORF). A nucleic acid sequence of interest comprising an ORF of interest may include the N-terminal methionine ATG codon, and a stop codon at the carboxy end, of the ORF, thus ATG-ORF-stop. Frequently, the nucleic acid molecule comprising the sequence of interest will include translation initiation sequences (tis) that may be located upstream of the ATG that allow expression of the gene, thus tis-ATG-ORF-stop. Constructs of this sort allow expression of an ORF as a protein that contains the same amino and carboxy amino acids as in the native, uncloned, protein. When such a construct is fused in-frame with an amino-terminal protein tag, e.g., GST, the tag will have its own tis, thus tis-ATG-tag-tis-ATG-ORF-stop, and the bases comprising the tis of the ORF will be translated into amino acids between the tag and the ORF. In addition, some level of translation initiation may be expected in the interior of the mRNA (i.e., at the ORF's ATG and not the tag's ATG) resulting in a certain amount of native protein expression contaminating the desired protein.

DNA (lower case): tis1-atg-tag-tis2-atg-orf-stop

RNA (lower case, italics): tis1-atg-tag-tis2-atg-orf-stop

Protein (upper case): ATG-TAG-TIS2-ATG-ORF (tis1 and stop are not translated)+contaminating ATG-ORF (translation of ORF beginning at tis2).

Using the methods disclosed herein, one skilled in the art can construct a vector containing a nucleic acid sequence encoding a polypeptide having a detectable activity (e.g., β-lactamase activity) adjacent to a recombination site permitting the in frame fusion of a nucleic acid sequence encoding a polypeptide having a detectable activity (e.g., β-lactamase activity) to the C- and/or N-terminus of the ORF of interest.

Given the ability to rapidly create a number of clones in a variety of vectors, there is a need in the art to maximize the number of ways a single cloned ORF can be expressed without the need to manipulate the construct itself. The present invention meets this need by providing materials and methods for the controlled expression of a C- and/or N-terminal fusion to a target ORF using one or more suppressor tRNAs to suppress the termination of translation at a stop codon. Thus, the present invention provides materials and methods in which a gene construct is prepared flanked with recombination sites.

The construct may be prepared with a sequence coding for a stop codon at the C-terminus of the ORF encoding the protein of interest. In some embodiments, a stop codon can be located adjacent to the ORF, for example, within the recombination site flanking the gene or at or near the 3′ end of the sequence of interest before a recombination site. The target gene construct can be transferred through recombination to various vectors that can provide various C-terminal or N-terminal tags (e.g., GFP, GST, His Tag, GUS, etc.) to the ORF of interest. In a particular embodiment of the invention, an ORF encoding a polypeptide of interest may be inserted into a vector comprising a nucleic acid sequence encoding a polypeptide having β-lactamase activity. When the stop codon is located at the carboxy terminus of the ORF, expression of the ORF with a “native” carboxy end amino acid sequence occurs under non-suppressing conditions (i.e., when the suppressor tRNA is not expressed) while expression of the ORF as a carboxy fusion protein occurs under suppressing conditions. Those skilled in the art will recognize that any suppressors and any codons could be used in the practice of the present invention. Suppressors may insert any amino acid at the position corresponding to the stop codon, for example, Ala, Arg, Asn, Asp, Cys, Gln, Glu, Gly, His, Ile, Leu, Lys, Met, Phe, Pro, Ser, Thr, Trp, Tyr, or Val may be inserted. In some embodiments, serine may be inserted.

III.D. Expressway™ and Tag-On-Demand™ Systems

In some embodiments, the invention relates to, or uses as an assay, Invitrogen's Expressway™ and Tag-On-Demand™ systems.

III.D. 1 .Expressway™

Expressway™ systems are described in detail in the following Manufacturer's Instruction Manuals for these products, all of which are incorporated by reference:

1. Expressway™ In Vitro Protein Synthesis System Manual, Version C, Apr. 11, 2003;

2. Expressway™ Linear Expression System Manual, Version A, 26 Sep. 2003;

3. Expressway™ Linear Expression System with TOPO® Tools Technology, Version A, 26 Sep. 2003;

4. Expressway Plus Expression System Manual, Version A, 26 Sep. 2003; and

5. Expressway Plus Expression System with Lumio Technology Manual, Version B, 27 Feb. 2004.

These manuals can be found on-line at the following respective web addresses:

1. www.invitrogen.com/content/sfs/manuals/expressway_man.pdf)

2. www.invitrogen.com/content/sfs/manuals/expresswaylinear_man.pdf;

3. www.invitrogen.com/content/sfs/manuals/expresswaylinearwithtopotools_man.pdf;

4. www.invitrogen.com/content/sfs/manuals/expresswayplus_man.pdf;

5. www.invitrogen.com/content/sfs/manuals/expresswayplus_lumio_man.pdf;

Two components of Invitrogen's E. coli expression systems, the ExpresswayTM Systems, are a crude cell-free S30 extract and a translation buffer. The S30 extract contains the majority of soluble translational components including initiation, elongation and termination factors, ribosomes and tRNAs from intact cells. The translation buffer contains energy sources such as ATP and GTP, energy regenerating components such as phosphoenol pyruvate/pyruvate kinase, acetyl phosphate/acetate kinase or creatine phosphate/ creatine kinase and a variety of other important co-factors (Zubay, Ann. Rev. Genet. 7:267-87, 1973; Pelham and Jackson, Eur J Biochem. 67:247, 1976; and Erickson and Blobel, Methods Enzymol. 96;38-50, 1983).

The Expressway™ Plus Expression System utilizes a coupled transcription and translation reaction to synthesize active recombinant protein. The Expressway™ Plus System provides all the components for cell-free protein production. The kit includes an E. coli extract containing the cellular machinery required to drive transcription and translation. The IVPS Plus reaction buffer is also included in the kit and contains the required amino acids (except methionine) and an ATP regenerating system for energy. The reaction buffer, methionine, T7 Enzyme Mix, and DNA template of interest, operably linked to a T7 promoter, are mixed with the E. coli extract. As the DNA template is transcribed, the 5′ end of the mRNA is bound by ribosomes and undergoes translation as the 3′ end of the template is still being transcribed.

The Expressway™ Linear Expression System is used for rapid high-yield in vitro expression from linear DNA templates. The system uses an E. coli extract optimized for expression of full-length, active protein from linear templates. As a result, linear templates are more stable during transcription and translation, resulting in higher yields of properly folded products. In the Expressway™ Linear Expression System, at least two options are available for generating T7 promoter-driven templates. The Expressway™ Linear Expression Kit can be used to express PCR templates generated from a plasmid containing the appropriate elements for expression (T7 promoter, ribosome binding site, T7 termination sequence). The Expressway™ Linear Expression Kit with TOPO® Tools includes a 5′ and 3′ element that can be operably joined to a PCR product. The 5′ element contains a T7 promoter, ribosome binding site, and start codon. The 3′ element contains a V5 epitope tag followed by a 6×His region and a T7 terminator. The TOPO® Tools elements are joined to the PCR product in a TOPO® ligation reaction and then amplified by PCR.

The Expressway™ Plus Expression System with Lumio™ Technology kit includes IVPS Lumio™ E. coli Extract, IVPS Plus E. coli Reaction Buffer, RNase A, T7 Enzyme Mix, Methionine, reaction tubes, pEXP3-DEST vector, a control plasmid, and a Lumio™ Green Detection Kit or components thereof. See Keppetipola et al., Rapid Detection of in vitro expressed proteins using Lumio™ Technology. Focus 25.3:7, 2003.

III.D.2.Tag-On-Demand™

An in vivo Tag-On-Demand™ Suppressor Supernatant system is available in a form for use with cultured cells, such as mammalian cells. In this system, a suppressor supernatant is added to cells into which is transfected an expression construct in which a gene or gene fragment of interest is separated by a stop codon from a nucleotide sequence encoding a polypeptide tag. When suppression occurs, read-through of the stop codon takes place and the expressed protein comprises the polypeptide tag. In this system for mammalian cells, the Tag-On-Demand™ Suppressor Supernatant comprises a replication-incompetent adenovirus containing the human tRNA-Ser suppressor gene. See Capone et al., Amber, ochre and opal suppressor tRNA genes derived from a human serine tRNA gene. EMBO J. 4:213, 1985). The Tag-On-Demand™ Suppressor Supernatant Instruction Manual, which is hereby incorporated by reference, can be found at invitrogen.com/content/sfs/manuals/tagondemand_sup ernatant_man.pdf. The Tag-On-Demand™ Gateway® Vector Instruction Manual, also hereby incorporated by reference, can be found at invitrogen.com/content/sfs/manuals/tagondemand_vectors_man.pdf.

In the Tag-On-Demand™ Suppressor Supernatant system, both tRNA generation and protein synthesis take place in vivo, i.e., within cells. This is done in part by introducing a gene encoding a suppressor tRNA into cells. This is in contrast to the present invention, wherein protein synthesis takes place in vitro and suppressor tRNA molecules, or extracts comprising suppressor tRNA molecules, are added to an in vitro protein synthesis system. Moreover, unlike the present invention, the Tag-On-Demand™ Suppressor Supernatant system does not involve codon bias and rare codon tRNAs.

III.E. Codon Bias

E. coli, like other organisms, has a species-specific pattern of codon usage (Sharp et al, 1988). In other words, a correlation exists between the abundance of certain tRNAs in E. coli and the cognate codons to which they correspond (Berg and Kurland, 1997). Codons that occur at low frequency in E. coli have been determined by examining sets of E. coli genes; these include the following: AGG-Arg; AGA-Arg; CGA-Arg; CUA-Leu; AUA-Ile; and CCC-Pro (de Boer and Kastelein, 1986). The lower frequency of these codons has deleterious affects on expression of genes having them in large numbers or clusters, mainly due to misincorporation or frame-shifting (McNulty et al, 2003). Forced over-expression of such genes is not possible because of depletion of endogenous tRNA pools.

Different methods have been developed for overcoming codon bias in E. coli. An in vivo approach has been the construction of different E. coli strains that contain extra copies of rare E. coli tRNA genes. For example, Stratagene's BL21-CodonPlus® and Novagen's Rosetta™ strains over-express different combinations of rare codon tRNA genes.

Patent documents that relate to rare codons include WO 00/44926, “High Level Expression of a Heterologous Protein Having Rare Codons”; and WO 00/36123, “Enhanced Expression of Heterologous Proteins in Recombinant Bacteria Through Reduced Growth Temperature and Co-Expression of Rare tRNA's”

IV. Cloning and Expression

IV.A. Definitions

Target Nucleic Acid Molecule: As used herein, the phrase “target nucleic acid molecule” refers to a nucleic acid segment of interest, preferably nucleic acid that is to be acted upon using the compounds and methods of the present invention. Such target nucleic acid molecules may contain one or more (e.g., two, three, four, five, seven, ten, twelve, fifteen, twenty, thirty, fifty, etc.) genes or one or more portions of genes.

Insert Donor: As used herein, the phrase “Insert Donor” refers to one of the two parental nucleic acid molecules (e.g., RNA or DNA) of the present invention that carries an insert. The Insert Donor molecule comprises the insert flanked on both sides with recombination sites. The Insert Donor can be linear or circular. In one embodiment of the invention, the Insert Donor is a circular nucleic acid molecule, optionally supercoiled, and further comprises a cloning vector sequence outside of the recombination signals. When a population of inserts or population of nucleic acid segments are used to make the Insert Donor, a population of Insert Donors result and may be used in accordance with the invention. In certain portions of the present description, the term “Insert Donor” is used interchangeably with, and should be considered to have the same meaning as, the term “Entry Vector” (or “pENTR”).

Insert: As used herein, the term “insert” refers to a desired nucleic acid segment that is a part of a larger nucleic acid molecule. In many instances, the insert will be introduced into the larger nucleic acid molecule. In most instances, the insert will be flanked by recombination sites, topoisomerase sites and/or other recognition sequences (e.g., at least one recognition sequence will be located at each end). In certain embodiments, however, the insert will only contain a recognition sequence on one end.

Product: As used herein, the term “Product” refers to one the desired daughter molecules comprising the A and D sequences that is produced after the second recombination event during the recombinational cloning process. The Product contains the nucleic acid that was to be cloned or subcloned. In accordance with the invention, when a population of Insert Donors are used, the resulting population of Product molecules will contain all or a portion of the population of Inserts of the Insert Donors and often will contain a representative population of the original molecules of the Insert Donors.

Byproduct: As used herein, the term “Byproduct” refers to a daughter molecule (a new clone produced after the second recombination event during the recombinational cloning process) lacking the segment that is desired to be cloned or subcloned.

Cointegrate: As used herein, the term “Cointegrate” refers to at least one recombination intermediate nucleic acid molecule of the present invention that contains both parental (starting) molecules. Cointegrates may be linear or circular. RNA and polypeptides may be expressed from cointegrates using an appropriate host cell strain, for example E. coli DB3.1 (particularly E. coli LIBRARY EFFICIENCY® DB3.1™ Competent Cells), and selecting for both selection markers found on the cointegrate molecule.

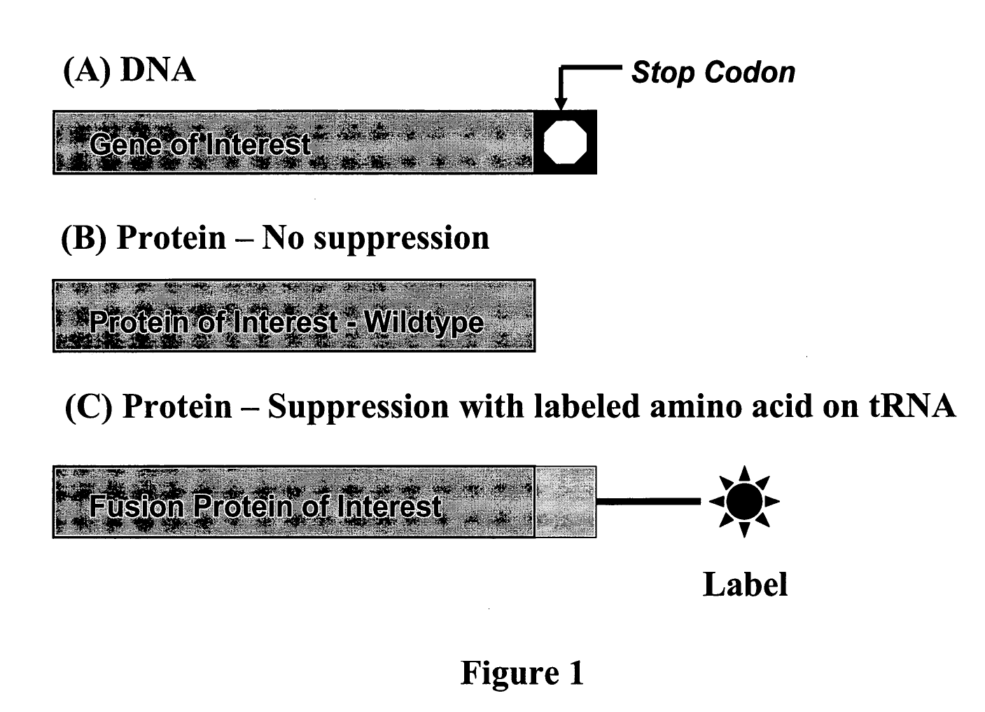

Recognition Sequence: As used herein, the phrase “recognition sequence” or “recognition site” refers to a particular sequence to which a protein, chemical compound, DNA, or RNA molecule (e.g., restriction endonuclease, a modification methylase, topoisomerases, or a recombinase) recognizes and binds. In some embodiments of the present invention, a recognition sequence may refer to a recombination site or topoisomerases site. For example, the recognition sequence for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Other examples of recognition sequences are the attB, attP, attL, and attR sequences, which are recognized by the recombinase enzyme λ Integrase. attB is an approximately 25 base pair sequence containing two 9 base pair core-type Int binding sites and a 7 base pair overlap region. attP is an approximately 240 base pair sequence containing core-type Int binding sites and arm-type Int binding sites as well as sites for auxiliary proteins integration host factor (IHF), FIS and excisionase (Xis) (see Landy, Current Opinion in Biotechnology 3:699-707 (1993)). Such sites may also be engineered according to the present invention to enhance production of products in the methods of the invention. For example, when such engineered sites lack the P1 or H1 domains to make the recombination reactions irreversible (e.g., attR or attp), such sites may be designated attR′ or attP′ to show that the domains of these sites have been modified in some way.

Recombination Proteins: As used herein, the phrase “recombination proteins” includes excisive or integrative proteins, enzymes, co-factors or associated proteins that are involved in recombination reactions involving one or more recombination sites (e.g., two, three, four, five, seven, ten, twelve, fifteen, twenty, thirty, fifty, etc.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Examples of recombination proteins include, but are not limited to, histonelike proteins (IHF, HU, etc.), Cre, Int,, Xis, Flp, Fis, Hin, Gin, ΦC31, Cin, Tn3 resolvase, TndX, XerC, XerD, TnpX, Hjc, SpCCE1, and ParA.

Recombinases: As used herein, the term “recombinases” is used to refer to the protein that catalyzes strand cleavage and re-ligation in a recombination reaction. Site-specific recombinases are proteins that are present in many organisms (e.g., viruses and bacteria) and have been characterized as having both endonuclease and ligase properties. These recombinases (along with associated proteins in some cases) recognize specific sequences of bases in a nucleic acid molecule and exchange the nucleic acid segments flanking those sequences. The recombinases and associated proteins are collectively referred to as “recombination proteins” (see, e.g., Landy, A., Current Opinion in Biotechnology 3:699-707 (1993)).

Numerous recombination systems from various organisms have been described. See, e.g., Hoess, et al., Nucleic Acids Research 14(6):2287 (1986); Abremski, et al., J. Biol. Chem. 261(1):391 (1986); Campbell, J. Bacteriol. 174(23):7495 (1992); Qian, et al., J. Biol. Chem. 267(11):7794 (1992); Araki, et al., J. Mol. Biol. 225(1):25 (1992); Maeser and Kahnmann, Mol. Gen. Genet. 230:170-176) (1991); Esposito, et al., Nucl. Acids Res. 25(18):3605 (1997). Many of these belong to the integrase family of recombinases (Argos, et al., EMBO J. 5:433-440 (1986); Voziyanov, et al., Nucl. Acids Res. 27:930 (1999)). Perhaps the best studied of these are the Integrase/att system from bacteriophage λ (Landy, A. Current Opinions in Genetics and Devel. 3:699-707 (1993)), the Cre/loxP system from bacteriophage P1 (Hoess and Abremski (1990) In Nucleic Acids and Molecular Biology, vol. 4. Eds.: Eckstein and Lilley, Berlin-Heidelberg: Springer-Verlag; pp. 90-109), and the FLP/FRT system from the Saccharomyces cerevisiae 2 μ circle plasmid (Broach, et al., Cell 29:227-234 (1982)).

Recombination Site: A used herein, the phrase “recombination site” refers to a recognition sequence on a nucleic acid molecule that participates in an integration/recombination reaction by recombination proteins. Recombination sites are discrete sections or segments of nucleic acid on the participating nucleic acid molecules that are recognized and bound by a site-specific recombination protein during the initial stages of integration or recombination. For example, the recombination site for Cre recombinase is loxP, which is a 34 base pair sequence comprised of two 13 base pair inverted repeats (serving as the recombinase binding sites) flanking an 8 base pair core sequence (see FIG. 1 of Sauer, B., Curr. Opin. Biotech. 5:521-527 (1994)). Other examples of recombination sites include the attB, attP, attL, and attR sequences described in U.S. patent application Ser. No. 09/517,466, filed Mar. 2, 2000, and Ser. No. 09/732,914, filed Aug. 14, 2003, and in U.S. patent publication No. 2002-0007051-A1—all of which are specifically incorporated herein by reference in their entireties—and mutants, fragments, variants and derivatives thereof, which are recognized by the recombination protein λ Int and by the auxiliary proteins integration host factor (IHF), FIS and excisionase (Xis) (see Landy, Curr. Opin. Biotech. 3:699-707 (1993)).

Recombination sites may be added to molecules by any number of known methods. For example, recombination sites can be added to nucleic acid molecules by blunt end ligation, PCR performed with fully or partially random primers, or inserting the nucleic acid molecules into an vector using a restriction site flanked by recombination sites. See, e.g., U.S. Pat. Nos. 5,888,732; 6,143,557; 6,171,861; 6,270,969; 6,277,608; and 6,720,140; the disclosures of all of which are incorporated herein by reference in their entireties.

Topoisomerase recognition site. As used herein, the term “topoisomerase recognition site” or “topoisomerase site” means a defined nucleotide sequence that is recognized and bound by a site specific topoisomerase. For example, the nucleotide sequence 5′-(C/T)CCTT-3′ is a topoisomerase recognition site that is bound specifically by most poxvirus topoisomerases, including vaccinia virus DNA topoisomerase I, which then can cleave the strand after the 3′-most thymidine of the recognition site to produce a nucleotide sequence comprising 5′-(C/T)CCTT-PO₄-TOPO, i.e., a complex of the topoisomerase covalently bound to the 3′ phosphate through a tyrosine residue in the topoisomerase (see Shuman, J. Biol. Chem. 266:11372-11379, 1991; Sekiguchi and Shuman, Nucl. Acids Res. 22:5360-5365, 1994; each of which is incorporated herein by reference; see, also, U.S. Pat. No. 5,766,891; PCT/US95/16099; and PCT/US98/12372, all of which are also incorporated herein by reference in their entireties). In comparison, the nucleotide sequence 5′-GCAACTT-3′ is the topoisomerase recognition site for type IA E. coli topoisomerase III.

Recombinational Cloning: As used herein, the phrase “recombinational cloning” refers to a method, such as that described in U.S. Pat. Nos. 5,888,732; 6,143,557; 6,171,861; 6,270,969; 6,277,608; and 6,720,140 (the disclosures of all of which are incorporated herein by reference in their entireties), whereby segments of nucleic acid molecules or populations of such molecules are exchanged, inserted, replaced, substituted or modified, in vitro or in vivo. In many instances, the cloning method is an in vitro method.

Cloning systems that utilize recombination at defined recombination sites have been previously described in U.S. Pat. Nos. 5,888,732; 6,143,557; 6,171,861; 6,270,969; 6,277,608; and 6,720,140; in pending U.S. application Ser. No. 09/517,466 filed Mar. 2, 2000; and in published U.S. application no. 2002 0007051-A1, all assigned to the Invitrogen Corporation, Carlsbad, Calif., the disclosures of which are specifically incorporated herein in their entirety. In brief, the GATEWAYS Cloning System (available commercially from Invitrogen Corporation) described in these patents and applications utilizes vectors that contain at least one recombination site to clone desired nucleic acid molecules in vivo or in vitro. In some embodiments, the system utilizes vectors that contain at least two different site-specific recombination sites that may be based on the bacteriophage lambda system (e.g., att1 and att2) that are mutated from the wild-type (att0) sites. Each mutated site has a unique specificity for its cognate partner att site (i.e., its binding partner recombination site) of the same type (for example attB1 with attP1, or attL1 with attR1) and will not cross-react with recombination sites of the other mutant type or with the wild-type attO site. Different site specificities allow directional cloning or linkage of desired molecules thus providing desired orientation of the cloned molecules. Nucleic acid fragments flanked by recombination sites are cloned and subcloned using the GATEWAY® system by replacing a selectable marker (for example, ccdB) flanked by att sites on the recipient plasmid molecule, sometimes termed the Destination Vector. Desired clones are then selected by transformation of a ccdb sensitive host strain and positive selection for a marker on the recipient molecule. Similar strategies for negative selection (e.g., use of toxic genes) can be used in other organisms such as thymidine kinase (TK) in mammals and insects.

Mutating specific residues in the core region of the att site can generate a large number of different att sites. As with the attl and att2 sites utilized in GATEWAY®, each additional mutation potentially creates a novel att site with unique specificity that will recombine only with its cognate partner att site bearing the same mutation and will not cross-react with any other mutant or wild-type att site. Novel mutated att sites (e.g., attB 1-10, attP 1-10, attR 1-10 and attL 1-10) are described in previous patent application Ser. No. 09/517,466, filed Mar. 2, 2000, which is specifically incorporated herein by reference. Other recombination sites having unique specificity (i.e., a first site will recombine with its corresponding site and will not recombine or not substantially recombine with a second site having a different specificity) may be used to practice the present invention. Examples of suitable recombination sites include, but are not limited to, loxP sites; loxP site mutants, variants or derivatives such as loxP511 (see U.S. Pat. No. 5,851,808); frt sites; frt site mutants, variants or derivatives; dif sites; dif site mutants, variants or derivatives; psi sites; psi site mutants, variants or derivatives; cer sites; and cer site mutants, variants or derivatives.

Selectable Marker: As used herein, the phrase “selectable marker” refers to a nucleic acid segment that allows one to select for or against a molecule (e.g., a replicon) or a cell that contains it and/or permits identification of a cell or organism that contains or does not contain the nucleic acid segment. Frequently, selection and/or identification occur under particular conditions and do not occur under other conditions.

Markers can encode an activity, such as, but not limited to, production of RNA, peptide, or protein, or can provide a binding site for RNA, peptides, proteins, inorganic and organic compounds or compositions and the like. Examples of selectable markers include but are not limited to: (1) nucleic acid segments that encode products that provide resistance against otherwise toxic compounds (e.g., antibiotics); (2) nucleic acid segments that encode products that are otherwise lacking in the recipient cell (e.g., tRNA genes, auxotrophic markers); (3) nucleic acid segments that encode products that suppress the activity of a gene product; (4) nucleic acid segments that encode products that can be readily identified (e.g., phenotypic markers such as β-lactamase, β-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic (e.g., Diphtheria toxin) or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication fuictions, e.g., replication in certain hosts or host cell strains or under certain environmental conditions (e.g., temperature, nutritional conditions, etc.).

Selection and/or identification may be accomplished using techniques well known in the art. For example, a selectable marker may confer resistance to an otherwise toxic compound and selection may be accomplished by contacting a population of host cells with the toxic compound under conditions in which only those host cells containing the selectable marker are viable. In another example, a selectable marker may confer sensitivity to an otherwise benign compound and selection may be accomplished by contacting a population of host cells with the benign compound under conditions in which only those host cells that do not contain the selectable marker are viable. A selectable marker may make it possible to identify host cells containing or not containing the marker by selection of appropriate conditions. In one aspect, a selectable marker may enable visual screening of host cells to determine the presence or absence of the marker. For example, a selectable marker may alter the color and/or fluorescence characteristics of a cell containing it. This alteration may occur in the presence of one or more compounds, for example, as a result of an interaction between a polypeptide encoded by the selectable marker and the compound (e.g., an enzymatic reaction using the compound as a substrate). Such alterations in visual characteristics can be used to physically separate the cells containing the selectable marker from those not contain it by, for example, fluorescent activated cell sorting (FACS).

Multiple selectable markers may be simultaneously used to distinguish various populations of cells. For example, a nucleic acid molecule of the invention may have multiple selectable markers, one or more of which may be removed from the nucleic acid molecule by a suitable reaction (e.g., a recombination reaction). After the reaction, the nucleic acid molecules may be introduced into a host cell population and those host cells comprising nucleic acid molecules having all of the selectable markers may be distinguished from host cells comprising nucleic acid molecules in which one or more selectable markers have been removed (e.g., by the recombination reaction). For example, a nucleic acid molecule of the invention may have a blasticidin resistance marker outside a pair of recombination sites and a β-lactamase encoding selectable marker inside the recombination sites. After a recombination reaction and introduction of the reaction mixture into a cell population, cells comprising any nucleic acid molecule can be selected for by contacting the cell population with blasticidin. Those cell comprising a nucleic acid molecule that has undergone a recombination reaction can be distinguished from those containing an unreacted nucleic acid molecules by contacting the cell population with a fluorogenic β-lactamase substrate as described below and observing the fluorescence of the cell population. Optionally, the desired cells can be physically separated from undesirable cells, for example, by FACS.

Selection Scheme: As used herein, the phrase “selection scheme” refers to any method that allows selection, enrichment, or identification of a desired nucleic acid molecules or host cells containing them (in particular Product or Product(s) from a mixture containing an Entry Clone or Vector, a Destination Vector, a Donor Vector, an Expression Clone or Vector, any intermediates (e.g., a Cointegrate or a replicon), and/or Byproducts). In one aspect, selection schemes of the invention rely on one or more selectable markers. The selection schemes of one embodiment have at least two components that are either linked or unlinked during recombinational cloning. One component is a selectable marker. The other component controls the expression in vitro or in vivo of the selectable marker, or survival of the cell (or the nucleic acid molecule, e.g., a replicon) harboring the plasmid carrying the selectable marker. Generally, this controlling element will be a repressor or inducer of the selectable marker, but other means for controlling expression or activity of the selectable marker can be used. Whether a repressor or activator is used will depend on whether the marker is for a positive or negative selection, and the exact arrangement of the various nucleic acid segments, as will be readily apparent to those skilled in the art. In some embodiments, the selection scheme results in selection of, or enrichment for, only one or more desired nucleic acid molecules (such as Products). As defined herein, selecting for a nucleic acid molecule includes (a) selecting or enriching for the presence of the desired nucleic acid molecule (referred to as a “positive selection scheme”), and (b) selecting or enriching against the presence of nucleic acid molecules that are not the desired nucleic acid molecule (referred to as a “negative selection scheme”).

In one embodiment, the selection schemes (which can be carried out in reverse) will take one of three forms. The first, exemplified herein with a selectable marker and a repressor therefore, selects for molecules having segment D and lacking segment C. The second selects against molecules having segment C and for molecules having segment D. Possible embodiments of the second form would have a nucleic acid segment carrying a gene toxic to cells into which the in vitro reaction products are to be introduced. A toxic gene can be a nucleic acid that is expressed as a toxic gene product (a toxic protein or RNA), or can be toxic in and of itself. (In the latter case, the toxic gene is understood to carry its classical definition of “heritable trait.”)

Examples of such toxic gene products are well known in the art, and include, but are not limited to, restriction endonucleases (e.g., Dpnl, Nla3, etc.); apoptosis-related genes (e.g., ASKI or members of the bcl-2/ced-9 family); retroviral genes; including those of the human immunodeficiency virus (HIV); defensins such as NP-1; inverted repeats or paired palindromic nucleic acid sequences; bacteriophage lytic genes such as those from Φ174 or bacteriophage T4; antibiotic sensitivity genes such as rpsL; antimicrobial sensitivity genes such as pheS; plasmid killer genes' eukaryotic transcriptional vector genes that produce a gene product toxic to bacteria, such as GATA-1; genes that kill hosts in the absence of a suppressing function, e.g., kicB, ccdB, Φ174 E (Liu, Q., et al., Curr. Biol. 8:1300-1309 (1998)); and other genes that negatively affect replicon stability and/or replication. A toxic gene can alternatively be selectable in vitro, e.g., a restriction site.

Many genes coding for restriction endonucleases operably linked to inducible promoters are known, and may be used in the present invention (see, e.g., U.S. Pat. No. 4,960,707 (DpnI and DpnII); U.S. Pat. Nos. 5,082,784 and 5,192,675 (KpnI); U.S. Pat. No. 5,147,800 (NgoAIII and NgoAI); U.S. Pat. No. 5,179,015 (FspI and HaeIII): U.S. Pat. No. 5,200,333 (HaeII and TaqI); U.S. Pat. No. 5,248,605 (HpaII); U.S. Pat. Nos. 5,312,746 (Clal); 5,231,021 and 5,304,480 (XhoI and XhoII); U.S. Pat. No. 5,334,526 (AluI); U.S. Pat. No. 5,470,740 (NsiI); U.S. Pat. No. 5,534,428 (SstI/SacI); U.S. Pat. No. 5,202,248 (NcoI); U.S. Pat. No. 5,139,942 (NdeI); and U.S. Pat. No. 5,098,839 (PacI). (See also Wilson, G. G., Nucl. Acids Res. 19:2539-2566 (1991); and Lunnen, K. D., et al., Gene 74:25-32 (1988)).

In the second form, segment D carries a selectable marker. The toxic gene would eliminate transformants harboring the Vector Donor, Cointegrate, and Byproduct molecules, while the selectable marker can be used to select for cells containing the Product and against cells harboring only the Insert Donor.

The third form selects for cells that have both segments A and D in cis on the same molecule, but not for cells that have both segments in trans on different molecules. This could be embodied by a selectable marker that is split into two inactive fragments, one each on segments A and D.

The fragments are so arranged relative to the recombination sites that when the segments are brought together by the recombination event, they reconstitute a functional selectable marker. For example, the recombinational event can link a promoter with a structural nucleic acid molecule (e.g., a gene), can link two fragments of a structural nucleic acid molecule, or can link nucleic acid molecules that encode a heterodimeric gene product needed for survival, or can link portions of a replicon.

Site-Specific Recombinase: As used herein, the phrase “site-specific recombinase” refers to a type of recombinase that typically has at least the following four activities (or combinations thereof): (1) recognition of specific nucleic acid sequences; (2) cleavage of said sequence or sequences; (3) topoisomerase activity involved in strand exchange; and (4) ligase activity to reseal the cleaved strands of nucleic acid (see Sauer, B., Current Opinions in Biotechnology 5:521-527 (1994)).

Conservative site-specific recombination is distinguished from homologous recombination and transposition by a high degree of sequence specificity for both partners. The strand exchange mechanism involves the cleavage and rejoining of specific nucleic acid sequences in the absence of DNA synthesis (Landy, A. (1989) Ann. Rev. Biochem. 58:913-949).

Suppressor tRNA. As used herein, the phrase “suppressor tRNA” is used to indicate a tRNA molecule that results in the incorporation of an amino acid in a polypeptide in a position corresponding to a stop codon in the mRNA being translated.

Homologous Recombination: As used herein, the phrase “homologous recombination” refers to the process in which nucleic acid molecules with similar nucleotide sequences associate and exchange nucleotide strands. A nucleotide sequence of a first nucleic acid molecule that is effective for engaging in homologous recombination at a predefined position of a second nucleic acid molecule will therefore have a nucleotide sequence that facilitates the exchange of nucleotide strands between the first nucleic acid molecule and a defined position of the second nucleic acid molecule. Thus, the first nucleic acid will generally have a nucleotide sequence that is sufficiently complementary to a portion of the second nucleic acid molecule to promote nucleotide base pairing.

Homologous recombination requires homologous sequences in the two recombining partner nucleic acids but does not require any specific sequences. As indicated above, site-specific recombination that occurs, for example, at recombination sites such as att sites, is not considered to be “homologous recombination,” as the phrase is used herein.

Vector: As used herein, the term “vector” refers to a nucleic acid molecule (e.g., DNA) that provides a useful biological or biochemical property to an insert. Examples include plasmids, phages, autonomously replicating sequences (ARS), centromeres, and other sequences that are able to replicate or be replicated in vitro or in a host cell, or to convey a desired nucleic acid segment to a desired location within a host cell. A vector can have one or more recognition sites (e.g., two, three, four, five, seven, ten, etc. recombination sites, restriction sites, and/or topoisomerases sites) at which the sequences can be manipulated in a determinable fashion without loss of an essential biological function of the vector, and into which a nucleic acid fragment can be spliced in order to bring about its replication and cloning. Vectors can further provide primer sites (e.g., for PCR), transcriptional and/or translational initiation and/or regulation sites, recombinational signals, replicons, selectable markers, etc. Clearly, methods of inserting a desired nucleic acid fragment that do not require the use of recombination, transpositions or restriction enzymes (such as, but not limited to, uracil N-glycosylase (UDG) cloning of PCR fragments (U.S. Pat. Nos. 5,334,575 and 5,888,795, both of which are entirely incorporated herein by reference), T:A cloning, and the like) can also be applied to clone a fragment into a cloning vector to be used according to the present invention. The cloning vector can further contain one or more selectable markers (e.g., two, three, four, five, seven, ten, etc.) suitable for use in the identification of cells transformed with the cloning vector.

Subcloning Vector: As used herein, the phrase “subcloning vector” refers to a cloning vector comprising a circular or linear nucleic acid molecule that includes, in many instances, an appropriate replicon. In the present invention, the subcloning vector (segment D) can also contain functional and/or regulatory elements that are desired to be incorporated into the final product to act upon or with the cloned nucleic acid insert (segment A). The subcloning vector can also contain a selectable marker (e.g., DNA).

Vector Donor: As used herein, the phrase “Vector Donor” refers to one of the two parental nucleic acid molecules (e.g., RNA or DNA) of the present invention that carries the nucleic acid segments comprising the nucleic acid vector that is to become part of the desired Product. The Vector Donor comprises a subcloning vector D (or it can be called the cloning vector if the Insert Donor does not already contain a cloning vector) and a segment C flanked by recombination sites. Segments C and/or D can contain elements that contribute to selection for the desired Product daughter molecule, as described above for selection schemes. The recombination signals can be the same or different, and can be acted upon by the same or different recombinases. In addition, the Vector Donor can be linear or circular. In certain portions of the present description, the term “Vector Donor” is used interchangeably with, and should be considered to have the same meaning as, the term “Destination Vector” (or “pDEST”).

Adapter: As used herein, the term “adapter” refers to an oligonucleotide or nucleic acid fragment or segment (e.g., DNA) that comprises one or more recombination sites and/or topoisomerase site (or portions of such sites) that can be added to a circular or linear Insert Donor molecule as well as to other nucleic acid molecules described herein. When using portions of sites, the missing portion may be provided by the Insert Donor molecule. Such adapters may be added at any location within a circular or linear molecule, although the adapters are typically added at or near one or both termini of a linear molecule. Adapters may be positioned, for example, to be located on both sides (flanking) a particular nucleic acid molecule of interest. In accordance with the invention, adapters may be added to nucleic acid molecules of interest by standard recombinant techniques (e.g., restriction digest and ligation). For example, adapters may be added to a circular molecule by first digesting the molecule with an appropriate restriction enzyme, adding the adapter at the cleavage site and reforming the circular molecule that contains the adapter(s) at the site of cleavage. In other aspects, adapters may be added by homologous recombination, by integration of RNA molecules, and the like. Alternatively, adapters may be ligated directly to one or more terminus or both termini of a linear molecule thereby resulting in linear molecule(s) having adapters at one or both termini. In one aspect of the invention, adapters may be added to a population of linear molecules, (e.g., a cDNA library or genomic DNA that has been cleaved or digested) to form a population of linear molecules containing adapters at one terminus or both termini of all or substantial portion of said population.

Adapter-Primer: As used herein, the phrase “adapter-primer” refers to a primer molecule that comprises one or more recombination sites (or portions of such recombination sites) that can be added to a circular or to a linear nucleic acid molecule described herein. When using portions of recombination sites, the missing portion may be provided by a nucleic acid molecule (e.g., an adapter) of the invention. Such adapter-primers may be added at any location within a circular or linear molecule, although the adapter-primers may be added at or near one or both termini of a linear molecule. Such adapter-primers may be used to add one or more recombination sites or portions thereof to circular or linear nucleic acid molecules in a variety of contexts and by a variety of techniques, including but not limited to amplification (e.g., PCR), ligation (e.g., enzymatic or chemical/synthetic ligation), recombination (e.g., homologous or non-homologous (illegitimate) recombination) and the like.

IV.B. Host Cells

The invention also relates to host cells comprising one or more of the nucleic acid molecules invention containing one or more nucleic acid sequences encoding a polypeptide having a detectable activity and/or one or more other sequences of interest (e.g., two, three, four, five, seven, ten, twelve, fifteen, twenty, thirty, fifty, etc.). Representative host cells that may be used according to this aspect of the invention include, but are not limited to, bacterial cells, yeast cells, plant cells and animal cells. In particular embodiments, bacterial host cells include Escherichia spp. cells (particularly E. coli cells and most particularly E. coli strains DH10B, Stbl2, DH5α, DB3, DB3.1 (e.g., E. coli LIBRARY EFFICIENCY® DB3.1™ Competent Cells; Invitrogen Corporation, Carlsbad, Calif.), DB4, DB5, JDP682 and ccdA-over (see U.S. application Ser. No. 09/518,188, filed Mar. 2, 2000, and U.S. provisional Application No. 60/475,004, filed Jun. 3, 2003, by Louis Leong et al., entitled “Cells Resistant to Toxic Genes and Uses Thereof,” the disclosures of which are incorporated by reference herein in their entireties); Bacillus spp. cells (particularly B. subtilis and B. megaterium cells), Streptomyces spp. cells, Erwinia spp. cells, Klebsiella spp. cells, Serratia spp. cells (particularly S. marcessans cells), Pseudomonas spp. cells (particularly P. aeruginosa cells), and Salmonella spp. cells (particularly S. typhimurium and S. typhi cells). Suitable animal host cells include insect cells (most particularly Drosophila melanogaster cells, Spodoptera frugiperda Sf9 and Sf21 cells and Trichoplusa High-Five cells), nematode cells (particularly C. elegans cells), avian cells, amphibian cells (particularly Xenopus laevis cells), reptilian cells, and mammalian cells (most particularly NIH3T3, 293, CHO, COS, VERO, BHK and human cells). Suitable yeast host cells include Saccharomyces cerevisiae cells and Pichia pastoris cells. These and other suitable host cells are available commercially, for example, from Invitrogen Corporation, (Carlsbad, Calif.), American Type Culture Collection (Manassas, Va.), and Agricultural Research Culture Collection (NRRL; Peoria, Ill.).

Nucleic acid molecules to be used in the present invention may comprise one or more origins of replication (ORIs), and/or one or more selectable markers. In some embodiments, molecules may comprise two or more ORIs at least two of which are capable of functioning in different organisms (e.g., one in prokaryotes and one in eukaryotes). For example, a nucleic acid may have an ORI that functions in one or more prokaryotes (e.g., E. coli, Bacillus, etc.) and another that functions in one or more eukaryotes (e.g., yeast, insect, mammalian cells, etc.). Selectable markers may likewise be included in nucleic acid molecules of the invention to allow selection in different organisms. For example, a nucleic acid molecule may comprise multiple selectable markers, one or more of which functions in prokaryotes and one or more of which functions in eukaryotes.

Methods for introducing the nucleic acids molecules of the invention into the host cells described herein, to produce host cells comprising one or more of the nucleic acids molecules of the invention, will be familiar to those of ordinary skill in the art. For instance, the nucleic acid molecules of the invention may be introduced into host cells using well known techniques of infection, transduction, electroporation, transfection, and transformation. The nucleic acid molecules of the invention may be introduced alone or in conjunction with other nucleic acid molecules and/or vectors and/or proteins, peptides or RNAs. Alternatively, the nucleic acid molecules of the invention may be introduced into host cells as a precipitate, such as a calcium phosphate precipitate, or in a complex with a lipid. Electroporation also may be used to introduce the nucleic acid molecules of the invention into a host. Likewise, such molecules may be introduced into chemically competent cells such as E. coli. If the vector is a virus, it may be packaged in vitro or introduced into a packaging cell and the packaged virus may be transduced into cells. Thus nucleic acid molecules of the invention may contain and/or encode one or more packaging signal (e.g., viral packaging signals that direct the packaging of viral nucleic acid molecules). Hence, a wide variety of techniques suitable for introducing the nucleic acid molecules and/or vectors of the invention into cells in accordance with this aspect of the invention are well known and routine to those of skill in the art. Such techniques are reviewed at length, for example, in Sambrook, J., et al., Molecular Cloning, a Laboratory Manual, 2nd Ed., Cold Spring Harbor, N.Y.: Cold Spring Harbor Laboratory Press, pp. 16.30-16.55 (1989), Watson, J. D., et al., Recombinant DNA, 2nd Ed., New York: W.H. Freeman and Co., pp. 213-234 (1992), and Winnacker, E.-L., From Genes to Clones, N.Y.: VCH Publishers (1987), which are illustrative of the many laboratory manuals that detail these techniques and which are incorporated by reference herein in their entireties for their relevant disclosures.

V. Fusion Protein Elements

The fusion proteins of the invention may comprise one or more fusion protein elements. Such elements include, but are not limited to, the following optional fusion protein elements. Optional fusion protein elements may be inserted between the displayed polypeptide and the membrane polypeptide, upstream or downstream (amino proximal and carboxy proximal, respectively) of these and other elements, or within the displayed polypeptide and the membrane polypeptide. A person skilled in the art will be able to determine which optional element(s) should be included in a fusion protein of the invention, and in what order, based on the desired method of production or intended use of the fusion protein.

Detectable polypeptides or reporter proteins are optional fusion protein elements that either generate a detectable signal or are specifically recognized by a detectably labeled agent. Examples of the former class of detectable polypeptide are green fluorescent protein (GFP) and its mutants, D.s. red and its mutants, and phycoerythrin. Other examples of reporter proteins have enzymatic activity that can generate a signal, such as, for example, chloramphenicol acetyl transferase (CAT), luciferase, GUS, beta galactosidase, etc. Examples of the latter class include epitopes such as a “His tag” (6 contiguous His residues, a.k.a. 6×His), the “FLAG tag” the hemmaglutinin tag, and the c-myc epitope. These and other epitopes can be detected using labeled antibodies that are specific for the epitope. Several such antibodies are commercially available.

Attachment (support-binding) elements or purification tags are optionally included in fusion proteins and can be used to attach minicells displaying a fusion protein to a preselected surface or support. Examples of such elements include a “His tag,” which binds to surfaces that have been coated with nickel; streptavidin or avidin, which bind to surfaces that have been coated with biotin or “biotinylated” (see U.S. Pat. No. 4,839,293 and Airenne et al., Protein Expr. Purif. 17:139-145, 1999); and glutathione-s-transferase (GST), which binds to surfaces coated with glutathione (Kaplan et al., Protein Sci. 6:399-406, 1997; U.S. Pat. No. 5,654,176). Calmodulin and domains thereof and maltose binding protein and domains thereof can also be employed as purification tags.. Polypeptides that bind to lead ions have also been described (U.S. Pat. No. 6,111,079).

Spacers (a.k.a. linkers) are amino acid sequences that are optionally included in a fusion protein in between other portions of a fusion protein (e.g., between the membrane polypeptide and the displayed polypeptide, or between an optional fusion protein element and the remainder of the fusion protein). Spacers can be included for a variety of reasons. For example, a spacer can provide some physical separation between two parts of a protein that might otherwise interfere with each other via, e.g., steric hindrance.

VI. Protein Synthesis Systems

In vitro translation systems can include extracts of cells or organisms, and can be from prokaryotic or eukaryotic systems. For use in the present invention, eukaryotic extracts can be, for example, extracts of embryos (such as, for example, Drosophila embryos), reticulocyte lysates, or plant extracts, such as wheat germ extract. The preparation of these extracts is well known in the art of protein synthesis. The extracts may be isolated from a cell, such as a prokaryotic cell (including a bacterial cell such as E. coli) or a eukaryotic cell (including yeast cells, mammalian cells, C. elegans cells, wheat cells, and the like). The extracts comprise ribosomes as well as other components of the cell or organism, such that, when the extracts are provided with a translatable template and supplemented with one or more of a suitable energy source, amino acid(s), salt(s) buffer(s), reducing agent(s), NTPs, etc., and incubated under the appropriate conditions, one or more polypeptides can be synthesized. The protein synthesis systems disclosed and provided herein are not reconstituted systems, as individual extract components are not purfied and titrated. For example, an extract used in an in vitro system can have endogenous tRNAs in addition to tRNAs that may be supplemented using the methods and compositions described herein.

In some embodiments of the present invention, the organism or cells from which an extract is made can have an altered genome that has an altered complement of tRNA genes. That is, the organism or cell (i) has a higher or lower number of one or more endogenous tRNA genes and/or (ii) comprises one or more non-endogenous tRNA, such as synthetic tRNA genes and cloned DNA. The cloned DNA can be, by way of non-limiting example, complementary DNA (cDNA); gene fragments, e.g., open reading frames (ORFs); genomic DNA, and the like. In certain such embodiments, the altered complement of tRNA genes is a genome altered by a process selected from the group consisting of:

(a) addition or deletion of one or more copies of one or more endogenous tRNA genes, which may have a gene product selected from the group consisting of a rare codon tRNA and a suppressor tRNA;

(b) addition of one or more non-endogenous tRNA genes, which may be a tRNA gene selected from the group consisting of a mitochondrial tRNA gene, a tRNA gene from a chloroplast, a tRNA gene from a virus, and a cloned tRNA gene;

(c) addition of one or more mutant tRNA genes, which may be an expanded codon tRNA; and

(d) combinations of one or more of (a), (b) and (c).

In other embodiments, the genome of the organism or cells from which the extract for protein synthesis are unaltered. The present invention provides in vitro synthesis systems in which genetic manipulation of the extract producing cell is not required; rather, in these systems, the desired results (such as efficient stop codon suppression, or enhancement of translation of genes comprising codons that are rare (referreing to their occurrence in the genome of the organism from which the protein synthesis extract is made) can be achieved through supplementation of the extract with exogenous tRNAs or other reagents. This provides versatility, flexibility, and convenience of use to the system.

In addition to a ribosome containing extract from a cell or organism, the in vitro protein synthesis systems of the present invention preferably include: amino acids, salts (such as magnesium), a buffer, a reducing agent, and an energy source (for example PEP, PK, ATP, GTP,etc.) for generating energy for translation. Components of translation systems and optimization of their concentrations is available in the scientific literature on protein translation. Where an in vitro protein synthesis system also performs transcription (an IVTT system), nucleotides and an RNA polymerase are present.

In performing protein synthesis reactions, a template nucleic acid molecule is employed. The template nucleic acid molecule encodes an open reading frame. The template molecule can be RNA (which is directly translated) or DNA (which must first be transcribed to RNA). Where a DNA template is used for in vitro protein synthesis, it preferably has an RNA polymerase promoter recognized by the RNA polymerase of the in vitro transcription reaction operably linked to the sequence encoding the open reading frame.

Supplementation of IVPS System with one or more Rare Codon RNAs

The in vitro protein synthesis compositions of the invention can be supplemented with one or more rare codon tRNAs to improve the efficiency or yield of translation of a protein of interest. In the context of the invention, a “rare codon” is a codon whose frequency in an organism's genes with respect to the frequency of all codons encoding the same amino acid is 0.2 or less. Preferably, the frequency of use of a rare codon as a fraction of all pssible codons for a given amino acid is 0.1 or less. In the context of the translation systems of the invention, a codon is considered rare is it is rare in the genome of the organism from which the translation extract is taken. This is relevant because the abundance of a tRNA that recognizes a rare codon is likely to be very low. The low abundance of such “rare codon tRNAs” in the extract can reduce translation yield. For example, a human gene may use an E. coli rare codon at a much greater frequency than it is used in E. coli.

Thus, in the case of an E. coli translation extract, a rare codon occurs at low frequency in the genes of E. coli. Other organisms from which a protein synthesis extract can be made may have different rare codons.

Because of the ease of use and versatility of the in vitro supplementation of tRNA to an IVPS system, the invention also encompasses supplementing a protein synthesis reaction with tRNAs that do not fall within the definition of “rare” provided above. Depending on the gene or genes whose ORFs are to be synthesized and the source of the extract, it may be desirable to supplement an extract with one or more exogenous tRNAs whether or not they are rare, to increase the efficiency of translation, or, in the case of orthogonal tRNAs or RNAs charged with modified or nonnatural amino acids, to produce novel or useful translation products. Such methods and resulting translation products are within the scope of the invention.

As illustrated in the Examples, the in vitro protein synthesis systems of the present invention are able to greatly improve the efficiency of translation of a gene of a heterologous organism by adding exogenous rare codon tRNAs to the cell extract used in the translation system.

The supplemented tRNA genes can be cloned (for example, from the same or a different species the extract is made from) and either overexpressed and subsequently isolated from an organism. Alternatively a cloned rare codon tRNA can or in vitro transcribed and isolated. The isolated rare codon tRNAs can then be added to IVPS reactions. Just one or several rare codon tRNA genes can be introduced into a single organism for overexpression and isolation. When isolating rare codon tRNAs from an organism in which they are overexpressed, the rare codon tRNAs will in most cases be isolated along with the host organism's normally expressed tRNA. This preparation can be used to supplement the in vitro protein synthesis system, as the rare codon tRNA will be overrepresented (due to induced overexpression). The resulting supplementing tRNA preparation will thus have overrepresented rare codon tRNAs and “background” host cell tRNAs.

The addition of exogenous rare codon tRNAs is versatile, flexible, and rapidly performed. It does not require genetic manipulation of host strains and allows the user to adjust the rare codon tRNA content of the IVPS reaction depending on the codon usage of the gene of interest that is to be translated. The rare codon tRNAs can be added to the extract, for example, prior to the addition of a buffer that includes amino acids, salts, energy molecules etc., or can provided in the buffer, or can be added separately.

In preferred embodiments, the cells used for making a protein synthesis extract are E. coli, and the gene of interest is a mammalian gene. E coli tRNA genes (identified by letter) are well characterized (see, for Example, www.ncbi.nlm.nih.gov/genomes). Nonlimiting examples of rare codon genes of E. coli, are thrU, glyT, leuW, argU, ileX, and proL.

However, rare codon tRNA genes can be isolated from any appropriate organism. The extract used for protein synthesis is also not limited to E. coli or prokaryotic cells or organisms, but also can be from eukaryotic cell or organisms. The supplementing tRNAs need not be produced from tRNA genes of the same species used to make the extract, nor need the supplementing tRNAs be produced in cells of the same species used to make the extract.

The invention includes methods of producing proteins using in vitro synthesis systems supplemented with rare codon tRNAs. In one embodiment, a method is provided for making a protein from an RNA template that includes: adding a mixture of amino acids, one or more rare codon tRNAs, and at least one RNA template that encodes a polypeptide that includes at least one of the rare codons recognized by the one or more rare condon tRNAs; and incubating the mixture to produce a polypeptide encoded by at least one RNA template. The polypeptide can optionally be at least partially purified after synthesis, for example by gel purification, column chromatography, affinity capture, etc. The order or manner of adding reagents, including rare codon tRNAs, is not limiting.

In another embodiment, a method is provided for making a protein from an DNA template that includes: adding a mixture of amino acids, a mixture of ribonucleotides, an RNA polymerase, one or more rare codon tRNAs, and at least one RNA template that encodes a polypeptide that includes at least one of the rare codons recognized by the one or more rare condon tRNAs; and incubating the mixture to produce a polypeptide encoded by at least one RNA template. The polypeptide can optionally be at least partially purified after synthesis, for example by gel purification, column chromatography, affinity capture, etc. The IVTT reaction can take place in one or two steps, that is, buffer adjustments and reagent addition can occur after a first incubation period and a second incubation period can be performed subsequently at the same or a different temperature. The order or manner of adding reagents, including rare codon tRNAs, is not limiting.

Supplementation of IVPS System with One or more Suppressor tRNAs

The in vitro protein synthesis compositions of the invention may comprise or be supplemented with one or more suppressor tRNA molecules, such as, for example, one or more mitochondrial tRNA molecules, one or more tRNA molecules from a chloroplast, one or more tRNAs molecule from a virus, one or more synthetic tRNA molecules, and/or one or more tRNA molecules from a cloned suppressor tRNA gene. In preferred embodiments, a cloned suppressor tRNA gene is expressed in cells from which tRNA is then isolated. The resulting tRNA preparation that includes the suppressor tRNA gene is used to supplement protein synthesis reactions. A cloned suppressor tRNA gene can also be in vitro transcribed, and the resulting suppressor tRNA can be isolated and used to supplement protein synthesis reactions.

The present invention can be applied to cause suppressor tRNAs to insert a naturally-occurring or nonnaturally-occurring amino acid at a stop codon, followed further by an amino acid sequence coding for a fusion protein element, such as one or more of a reporter or detection protein, labeling tag, purification tag or protease cleavage site. One advantage of the invention is that any given gene or gene fragment can be expressed as a fusion protein with a detectably labeled tag (when suppressor tRNA is present), or as wildtype (non-tagged) protein. The wildtype form of the protein, lacking the tag, is free from any adverse affects the tag may have on its structure, activity or molecular interactions. The wildtype protein may be especially useful for in vitro applications. The tagged (detectably labeled) protein can be useful for applications in which protein transport and distribution are being studied or characterized, such as in a cell, tissue, organ or organism, i.e., in vivo applications. In preferred embodiments of the invention, the label of the tagged fusion protein is detectable in cells without disruption to any of the processes therein.

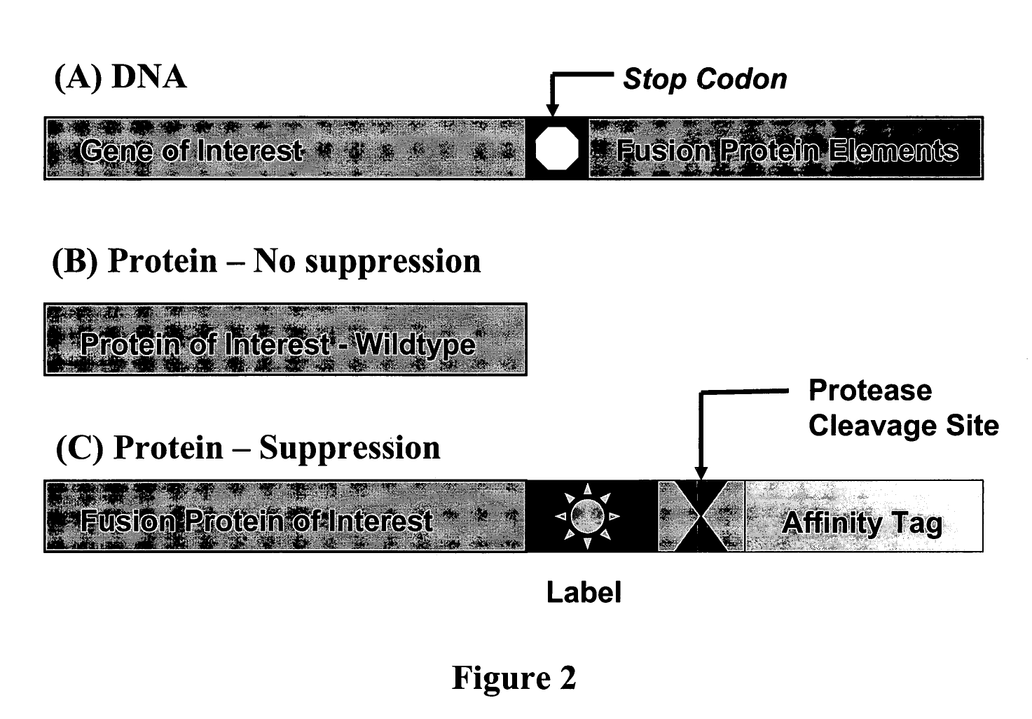

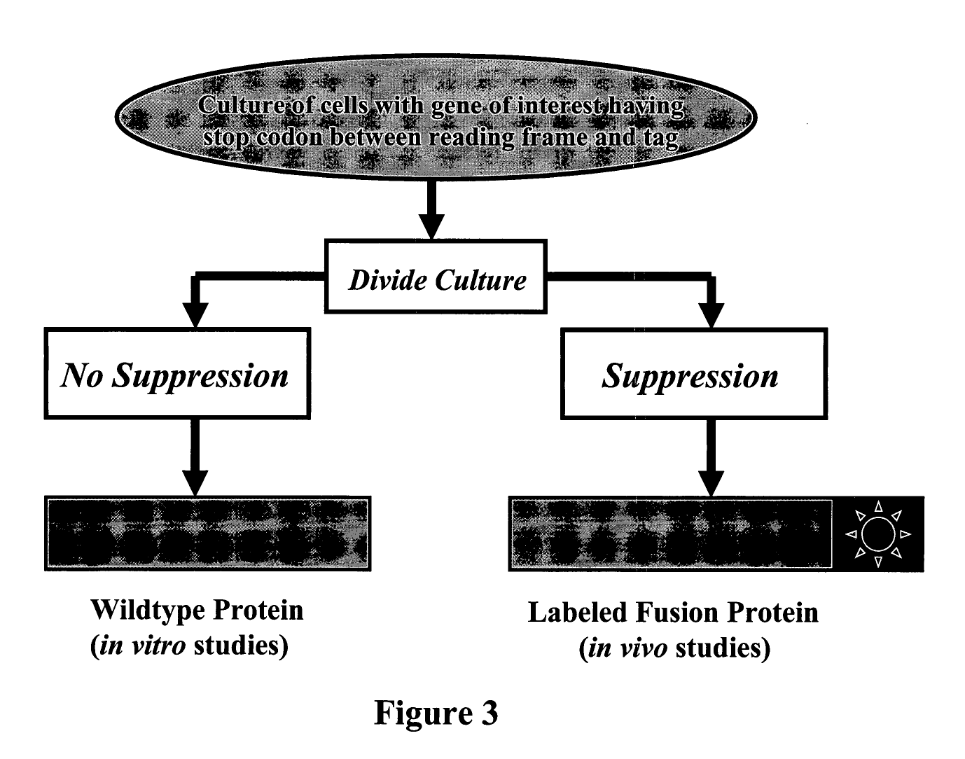

FIG. 2 depicts a protein made without stop codon suppression and versions of fusion proteins having polypeptide labels or tags that may be an amino acid sequence that binds, covalently or non-covalently, to a detectably labeled molecule. Moreover, additional elements besides the label can be added (for example, cleavage sites) using these methods.

As illustrated in the Examples, the in vitro protein synthesis systems of the present invention are able to allow readthrough of a stop codon by adding a suppressor tRNAs to the cell extract used in the translation system. In the illustrative embodiments, the extract is an E. coli S30 extract, and a suppressor tRNA (expressed from the cloned psul gene of phage T4) that recognizes the amber codon (UAG) is used. It is also possible to use a suppressor tRNA that recognizes the ochre codon (UAA) or the opal stop codon (UGA). It is also within the scope of the invention to include more than one suppressor tRNA in an in vitro synthesis. When using multiple suppressor tRNAs, the tRNAs can recognize and suppress one, two, or all three stop codons. In vitro synthesis systems can use RNA templates or DNA templates (in which case rNTPs and RNA polymerase are supplied for the transcription reaction).

The practice of the invention is not limited to E. coli or to prokaryotic translation systems. In vitro protein synthesis systems based on eukaryotic extracts (for example, Drosophila embryo extracts, wheat germ extracts, rabbit reticulocyte extracts) can also be supplemented with suppressor tRNAs.

The invention includes methods of synthesizing fusion proteins using in vitro synthesis systems supplemented with suppressor tRNAs. In one embodiment, a method is provided for making a protein from an RNA template that includes: adding a mixture of amino acids, one or more suppressor tRNAs, and at least one RNA template that has a first open reading frame that terminates in a stop codon that is suppressed by the one or more suppressor tRNAs added and a second open reading frame contiguous with and beginning immediately after the stop codon, such that suppression of the stop codon results in translation of a fuson protein comprising the first and second opend reading frames linked by an amino acid incorporated by the added suppressor tRNA. Preferably, a biochemical energy source for translation is also added to the extract. The suppressor tRNAs can be added to the extract, for example, prior to the addition of a buffer that includes amino acids, salts, energy molecules etc., or can provided in the buffer, or can be added separately.

The amino acids, suppressor tRNA, and RNA template are incubated to synthesize a fusion protein encoded by the RNA template. The protein can optionally be at least partially purified after synthesis, for example by gel purification, column chromatography, affinity capture, etc.

In another embodiment, a method is provided for making a protein from an DNA template that includes: adding a mixture of amino acids, one or more suppressor tRNAs, a mixture of ribonucleotides, an RNA polymerase, and at least one DNA template that has a first open reading frame that terminates in a stop codon that is suppressed by the one or more suppressor tRNAs added and a second open reading frame contiguous with and beginning immediately after the stop codon, such that suppression of the stop codon results in translation of a fuson protein comprising the first and second opend reading frames linked by an amino acid incorporated by the added suppressor tRNA. Preferably, an energy source for translation is also added to the extract. The order of addition of components is not limiting. One or more suppressor tRNAs can be provided in the extract, or in a buffer, or added to the IVPS as a single reagent.

The amino acids, suppressor tRNA, ribonucleotides, RNA polymerase, and DNA template are incubated to synthesize a fusion protein encoded by the DNA template. The protein can optionally be at least partially purified after synthesis, for example by gel purification, column chromatography, affinity capture, etc.

In a related embodiment the methods of the present invention can be used to suppress a stop codon during translation and insert a modified or nonnaturally-occurring amino acid into the protein where the protein would otherwise terminate. In these embodiments, in vitro protein synthesis systems are supplemented with suppressor tRNAs that are charged with modified or nonnaturally-occurring amino acids. For example, orthogonal suppressor tRNAs can incorporate labeled or unnatural amino acids into a polypeptide (see, for example, US20040265952A1). FIG. 1 depicts a scheme in which a label is covalently attached to a protein during protein synthesis. (See Gite et al., Ultrasensitive fluorescence-based detection of nascent proteins in gels. Anal Biochem. 279:218, 2000; Mamaev et al., Cell-free N-terminal protein labeling using initiator suppressor tRNA. Anal Biochem. 326:25, 2004.)

It is within the scope of the invention to combine features to produce new embodiments of the invention. In particular, the invention includes in vitro protein synthesis systems and methods that include the use of one or more rare or unconventional tRNAs (orthogonal or having modified or nonnatural amino acids) and one or more suppressor tRNAs. These systems and methods can be used to produce, for example, fusion proteins or labeled proteins at a greater yield or with greater efficiency due to supplementation of the reaction mixture with rare codon tRNAs.

Inhibiting the Activity of One or More Translation Termination Factors

Certain compositions of the invention may further, or alternatively, comprise or be supplemented with one or more additional components or compositions comprising one or more molecules that inhibit the activity of one or more translation termination factors, such as one or more antibodies that bind to and/or inhibit one or more translation termination factors.

Inhibition can be reversible or irreversible. Inhibition can be by any means, including binding to one or more translation termination factors to inhibit their interaction with the translation machinery, binding to molecules that bind a termination factor, cleaving, degrading, or denaturing a translation termination factor, or removing one or more termination factors from the translation reaction. For example, one or more factors that promote translation termination can be bound by one or more specific bindng partners, and either precipitated out of the translation solution or capture to a solid support. Inhibiting the activity of one or more translation termination factors can use multiple inhibitors, for example a cocktail of antibodies or other inhibitors.

In E. coli, for example, Release Factor 1 (RF1) promotes translation termination at UAA and UAG, and Release Factor 2 (RF2) promotes translation termination at UAA and UGA. Reagents that inhibit RF1 or RF2 activity can be used to increase stop codon suppression by a suppressor tRNA. One or more reagents used for this purpose can partially or essentially completely inhibit the termination-promoting activity of RF1, RF2, or both. Specific binding partners such as antibodies for Release Factors such as RF1, RF2, and eukaryotic release factor (eRF) can be used to deplete a translation mix of these factors and thereby inhibit termination, such as by enhancing suppression by suppressor tRNAs. Multiple inhibitors, such as multiple antibodies to a release factor, can be employed, for example, as an inhibitor cocktail.

In some embodiments, the IVPS composition includes an antibody that recognizes a translation termination factor, such as the E. coli Release Factor 1 (RF1). RF1 acts to terminate translation at the amber (UAG) and ochre (UAA) codons (Craigen et al. Recent advances in peptide chain termination. Mol. Microbiol. 4:861, 1990). It has been shown that depletion of RF1 increases the read-through at amber and ochre stop codons. In genetic studies, purified component systems, extracts containing temperature sensitive mutants of RF1, suppressor tRNAs more efficiently substitute their cognate amino acid at the amber or and ochre stop codons (respectively, Ryden et al., Mapping and complementation studies of the gene for release factor 1. J. Bacteriol. 168:1066, 1986; Shimizu et al., Cell-free translation reconstituted with purified components. Nat. Biotechnol. 19:751, 2001; and Short et al., Effects of release factor 1 on in vitro protein translation and the elaboration of proteins containing unnatural amino acids. Biochemistry 38:8808, 1999). The use of an antibody that binds to, and reduces or eliminates RF1 activity enhances suppression of termination at the amber codon. As described in the Examples, an antibody against RF1 improves suppression of stop codon. Antibodies to E. coli RF2 or the eukaryotic Release Factor, for use in eukaryotic translation systems, can also be used to enhance stop codon suppression.

The invention includes methods of synthesizing fusion proteins using in vitro synthesis systems supplemented with suppressor tRNAs. In one embodiment, a method is provided for making a protein from an RNA template that includes: adding a mixture of amino acids, one or more suppressor tRNAs, a reagent that inhibits the activity of a release factor (RF) and at least one RNA template that has a first open reading frame that terminates in a stop codon that is suppressed by the one or more suppressor tRNAs added and a second open reading frame contiguous with and beginning immediately after the stop codon, such that suppression of the stop codon results in translation of a fuson protein comprising the first and second opend reading frames linked by an amino acid incorporated by the added suppressor tRNA. The reagent that inhibits a release factor inhibits a release factor that normally (when not inhibited) promotes translation termination at the stop codon that links the two open reading frames of the fusion protein. Thus, it does not promote termination and allows suppression of the stop codon by the suppressor tRNA. Preferably, an energy source for translation is also added to the extract. The one or more release factor inhibitors can be added to the extract, to which additional reagents are subsequently added. Alternatively, an inhibitor or inhibitor cocktail can be added at the time other reagents are added to the protein synthesis reaction.

In another embodiment, a method is provided for making a protein from an DNA template that includes: adding a mixture of amino acids, one or more suppressor tRNAs, a reagent that inhibits the activity of an RF, a mixture of ribonucleotides, an RNA polymerase, and at least one DNA template that has a first open reading frame that terminates in a stop codon that is suppressed by the one or more suppressor tRNAs added and a second open reading frame contiguous with and beginning immediately after the stop codon, such that suppression of the stop codon results in translation of a fuson protein comprising the first and second opend reading frames linked by an amino acid incorporated by the added suppressor tRNA. The reagent that inhibits a release factor inhibits a release factor that normally (when not inhibited) promotes translation termination at the stop codon that links the two open reading frames of the fuision protein. Thus, it does not promote termination and allows suppression of the stop codon by the suppressor tRNA. Preferably, an energy source for translation is also added to the extract. The one or more release factor inhibitors can be added to the extract, to which additional reagents are subsequently added. Alternatively, an inhibitor or inhibitor cocktail can be added at the time other reagents are added to the protein synthesis reaction.

The amino acids, suppressor tRNA, ribonucleotides, RNA polymerase, and DNA template are incubated to synthesize a fusion protein encoded by the DNA template. The protein can optionally be at least partially purified after synthesis, for example by gel purification, column chromatography, affinity capture, etc.

The use of inhibitor of release factors and other translation termination factors can also be combined with the supplementation of the reaction mixture with rare codon tRNAs. Preferably but optionally, such methods include the addition of at least one suppressor tRNA to the translation reaction mixture.

VII. Kits

The invention provides kits for use in synthesizing proteins that comprise: an extract of a cell or organism, amino acids, and one or more preparations of one or more rare codon tRNAs. The extract, amino acids, and rare codon tRNAs (or orthogonal tRNAs, or tRNAs charged with modified or nonnatural amino acids) can be provided in separate containers. Alternatively, rare codon or unconventional tRNAs can be provided in the extract or can be provided in a solution or buffer that also comprises amino acids.

The kits can also supply an energy source for translation, and, optionally ribonucleotides which optionally can be provided in a general reaction buffer. RNA polymerase can also be supplied, preferably in a separated tube or vial.

The invention also provides kits for use in synthesizing proteins that comprise: an extract of a cell or organism, amino acids, and one or more preparations of one or more suppressor tRNAs. The extract, amino acids, and suppressor tRNAs can be provided in separate containers. The kits optionally but preferably can include one or more inhibitors of one or more translation termination factors. In preferred embodiments, a kit includes one or more antibodies that inhibit the activity of one or more Release Factors, such as RF1 or RF2. The inhibitors can be provided separately, in a reaction buffer, or in the extract.

The kits may also include one or more rare codon tRNAs, provided either in the extract, as an independent reagent, in a reaction buffer, or in combination with suppressor tRNAs.

The kits can also supply an energy source for translation, and, optionally, ribonucleotides and RNA polymerase.

The kits can also supply expression vectors for cloning sequences having open reading frames.

Kits according to these aspects of the invention may comprise one or more containers, which may contain one or more of the compositions of the present invention. In additional embodiments, the kits may comprise one or more additional components (which may be in the same or different containers) selected from the group consisting of one or more nucleic acid molecules (e.g., one or more nucleic acid molecules comprising one or more nucleic acid sequence encoding a polypeptide having a detectable activity) of the invention, one or more primers, one or more of the molecules and/or compounds and/or compositions of the invention, one or more polymerases, one or more reverse transcriptases, one or more recombination proteins (or other enzymes for carrying out the methods of the invention), one or more topoisomerases, one or more buffers, one or more detergents, one or more restriction endonucleases, one or more nucleotides, one or more terminating agents (e.g., ddNTPs), one or more transfection reagents, pyrophosphatase, and the like. Kits of the invention may also comprise written instructions for carrying out one or more methods of the invention.

The present invention also provides kits that contain components useful for conveniently practicing the methods of the invention. In one embodiment, a kit of the invention contains a first nucleic acid molecule, which comprises a nucleic acid sequence encoding a polypeptide having a detectable activity, and contains one or more topoisomerase recognition sites and/or one or more covalently attached topoisomerase enzymes. Nucleic acid molecules according to this aspect of the invention may further comprise one or more recombination sites. In some embodiments, the nucleic acid molecule comprises a topoisomerase-activated nucleotide sequence. The topoisomerase-charged nucleic acid molecule may comprise a 5′ overhanging sequence at either or both ends and, the overhanging sequences may be the same or different. Optionally, each of the 5′ termini comprises a 5′ hydroxyl group.

In one embodiment, a kit of the invention contains a first nucleic acid molecule, which comprises a nucleic acid sequence encoding a polypeptide having a detectable activity, and contains one or more recombination sites. Nucleic acid molecules according to his aspect of the invention may further comprise one or more topoisomerase sites and/or topoisomerase enzymes.

In addition, the kit can contain at least a nucleotide sequence (or complement thereof) comprising a regulatory element, which can be an upstream or downstream regulatory element, or other element, and which contains a topoisomerase recognition site at one or both ends. In particular embodiments, kits of the invention contain a plurality of nucleic acid molecules, each comprising a different regulatory element or other element, for example, a sequence encoding a tag or other detectable molecule or a cell compartmentalization domain. The different elements can be different types of a particular regulatory element, for example, constitutive promoters, inducible promoters and tissue specific promoters, or can be different types of elements including, for example, transcriptional and translational regulatory elements, epitope tags, and the like. Such nucleic acid molecules can be topoisomerase-activated, and can contain 5′ overhangs or 3′ overhangs that facilitate operatively covalently linking the elements in a predetermined orientation, particularly such that a polypeptide such as a selectable marker is expressible in vitro or in one or more cell types.

The kit also can contain primers, including first and second primers, such that a primer pair comprising a first and second primer can be selected and used to amplify a desired ds recombinant nucleic acid molecule covalently linked in one or both strands, generated using components of the kit. For example, the primers can include first primers that are complementary to elements that generally are positioned at the 5′ end of a generated ds recombinant nucleic acid molecule, for example, a portion of a nucleic acid molecule comprising a promoter element, and second primers that are complementary to elements that generally are positioned at the 3′ end of a generated ds recombinant nucleic acid molecule, for example, a portion of a nucleic acid molecule comprising a transcription termination site or encoding an epitope tag. Depending on the elements selected from the kit for generating a ds recombinant nucleic acid molecule covalently linked in both strands, the appropriate first and second primers can be selected and used to amplify a full length functional construct.

In another embodiment, a kit of the invention contains a plurality of different elements, each of which can comprise one or more recombination sites and/or can be topoisomerase-activated at one or both ends, and each of which can contain a 5′-overhanging sequence or a 3′-overhanging sequence or a combination thereof. The 5′ or 3′ overhanging sequences can be unique to a particular element, or can be common to plurality of related elements, for example, to a plurality of different promoter element. In particular embodiments, the 5′ overhanging sequences of elements are designed such that one or more elements can be operatively covalently linked to provide a useful function, for example, an element comprising a Kozak sequence and an element comprising a translation start site can have complementary 5′ overhangs such that the elements can be operatively covalently linked according to a method of the invention.

The plurality of elements in the kit can comprise any elements, including transcription or translation regulatory elements; elements required for replication of a nucleotide sequence in a bacterial, insect, yeast, or mammalian host cell; elements comprising recognition sequences for site specific nucleic acid binding proteins such as restriction endonucleases or recombinases; elements encoding expressible products such as epitope tags or drug resistance genes; and the like. As such, a kit of the invention provides a convenient source of different elements that can be selected depending, for example, on the particular cells that a construct generated according to a method of the invention is to be introduced into or expressed in. The kit also can contain PCR primers, including first and second primers, which can be combined as described above to amplify a ds recombinant nucleic acid molecule covalently linked in one or both strands, generated using the elements of the kit. Optionally, the kit further contains a site specific topoisomerase in an amount useful for covalently linking in at least one strand, a first nucleic acid molecule comprising a topoisomerase recognition site to a second (or other) nucleic acid molecule, which can optionally be topoisomerase-activated nucleic acid molecules or nucleotide sequences that comprise a topoisomerase recognition site.

In still another embodiment, a kit of the invention contains a first nucleic acid molecule, which comprises a nucleic acid sequence encoding a polypeptide having a detectable activity, and contains a topoisomerase recognition site and/or a recombination site at each end; a first and second PCR primer pair, which can produce a first and second amplification products that can be covalently linked in one or both strands, to the first nucleic acid molecule in a predetermined orientation according to a method of the invention.

Kits of the invention may further comprise (1) instructions for performing one or more methods described herein and/or (2) a description of one or more compositions described herein. These instructions and/or descriptions may be in printed form. For example, these instructions and/or descriptions may be in the form of an insert which is present in kits of the invention.

It will be understood by one of ordinary skill in the relevant arts that other suitable modifications and adaptations to the methods and applications described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

### Preparation of Plasmids and Strains Over-Expressing Rare Codon tRNA (rctRNA) Molecules

1.A. Rare Codon tRNA Plasmid Construction

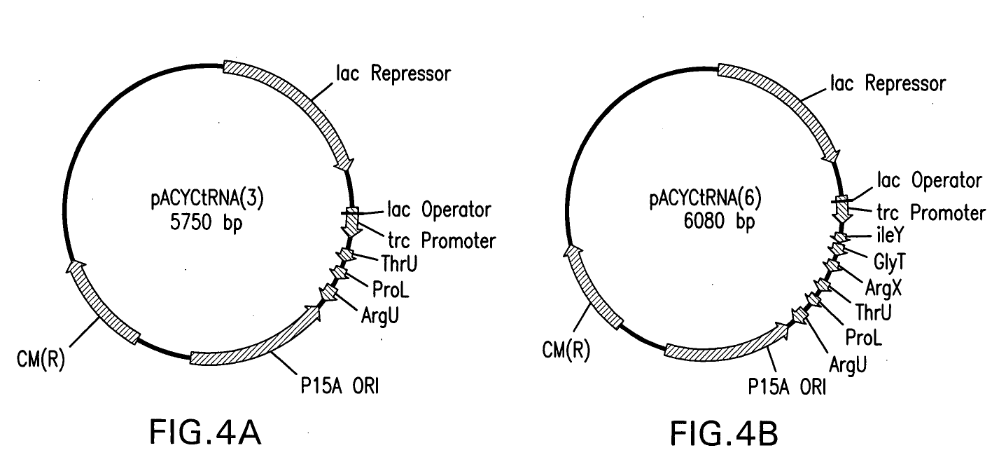

Plasmid pACYCtRNA(3) has 3 tRNA rare codon genes (Thr U, Pro L and Arg U) and pACYCtRNA(6) has 6 (Ile Y, Gly T, Arg X, Thr U, Pro L and Arg U) in a pACYC184 backbone (FIG. 4). Rare tRNA codons were cut from a pTrc His2 plasmid at the Hind III and Sph I sites and placed into the Hind III and Sph I sites over the tet site in the pACYC184 plasmid (New England Biolabs). Both pACYC cassette 1 and pACYC cassette 2 plasmids were transformed into BL2 1 Star™ E. Coli (Invitrogen).

The BL21 Star™, a host strain most commonly used for protein expression because the deletions of the Lon and ompT proteases increase protein stability (Phillips, Van Bogelen and Neidhardt, 1984). Another variant of the BL21 Star strain is the BL21™ Star pLysS, which provides decreased basal-level expression of heterologous genes and is used for expression of proteins that can cause growth inhibition in E. coli. Both the BL21 Star™ and BL21™ Star pLysS strains were used for over-expression of rare tRNAs from pACYCtRNA 3 and pACYCtRNA6. After comparing results from final yields of tRNA, the BL21 Star™ gave the best yield, and cell growth of E. coli did not appear to be compromised by tRNA overexpression.

1.B. Over-Expression of pACYCtRNA(3) and pACYCtRNA(6)

Five milliliter cultures were grown from one colony each of pACYC cassettes 1 and 2, transformed as above, for ˜8 hours using chloremphenicol resistance and were transferred to a 500 ml culture for growth overnight at 37° C. The O.D. of the 500 ml culture was determined the following day, and it was used to seed a 1000 ml culture at a starting O.D. of 0.05. The 1000 ml culture was grown at 37° C. for ˜2. 5 hours to an O.D. of 0.3. The culture was induced with lmM final concentration of IPTG and grown for 3 hrs at 30° C. The cells were harvested by centrifugation at 5000×g for 30 minutes. The supernatant was poured off, and pellets were frozen at 80° C.

1.C. Purification of rctRNA

1.C.1. Lysis and Cell Pellet Preparations of A19 E. coli Harboring pACYCsupT4

Based on the weight of the pellet (e.g. 10 g=30 ml), 3 volumes of bacterial lysis buffer [0.15 M NaCl, 10 mM Tris-HCl (pH 8.0], 1 mM EDTA and 0.1 mg/ml lysozyme) were used for resuspension. The resuspended cells were incubated for 20 minutes at room temperature before adding SDS to a final concentration of 1%. One volume of RNA lysis solution (Micro-to-Midi purification system RNA Lysis Solution, Invitrogen) plus 1% beta-mercaptoethanol (BME) was added, and lysis was completed by using a Polytron for 10 minutes at 4° C. (the Polytron was assembled and used in the hood. Alternatively, samples were lysed by passing the lysate through an 18-21-gauge needle 3-4 times). After lysis, 2 volumes of 3M sodium acetate pH 4.5 and 1 volume of chloroform were added. The solution was mixed well with forceps, transferred to centrifuge bottles and centrifuged for 20 minutes at 4,000 RPM. The supernatant was removed and the volume measured. In a beaker, 0.35 volume of isopropanol was added to precipitate large molecular weight RNA and genomic DNA. Solution was stirred with a pipette to help break-up the large precipitates before passing it over a layer of Miracloth (Calbiochem) into a new beaker. More isopropanol was added to a total of 0.7 volumes. The solution was placed at 4° C. for >30 minutes after which it was centrifuged at 5000 RPM at 4° C. for 30 minutes. The pellet was washed with 80% ethanol and centrifuged at 10,000 RPM at 4° C. for 15 minutes. The supernatant was removed and the pellet was dried overnight at 4° C.

1.C.2. tRNA Purification

The cell pellet from above was dissolved in 10 mM tris pH 8.0, 1 mM EDTA (TE), 250 mM NaCl pH 8. A 10 ml Q sepharose column was prepared by washing twice with RNase/DNase-free water and equilibrating in TE, 250 mM NaCl. A gradient for the column was set up on the FPLC. The column was washed with 4 column volumes of TE, 250 mM NaCl (Port A). TE 1M NaCl, pH 8.0 was placed in port B. A gradient was set-up for a target of 100% B. The sample was loaded by injection. One milliliter fractions were collected at 4° C.

Pool fractions were determined by running 5 μl of collected fractions (chosen according to chart peaks) on 10% TBE-Urea Novex gels. Gel samples were prepared by precipitating 5 μl of the 50 μl reactions in 20 μl of 100% acetone and incubated at 4° C. for >20 minutes. Reactions were pelleted and raised in 20 μl of 1×SDS loading buffer with BME and 5 μl was loaded on either 4-12% or 4-20% pre-poured NOVEX gradient gels. Gels were stained with Coomassie brilliant blue, dried and exposed to MR film. Gels were stained in 0.5 ug/ml ethidium bromide (EtBr)+1×TBE for 5 min. Fractions were pooled that did not contain genomic DNA and large RNA. After fractions were pooled, 1/10 volume of 3M sodium acetate (NaOAC)pH 4.5 and 0.4 volume isopropanol was added. The solution was mixed well and placed at 4° C. overnight.

The next day, the mixture was centrifuged at 10,000 RPM at 4° C. for 30 minutes, and the pellet was discarded. The supernatant containing the tRNA was collected to a new tube (or bottle). More isopropanol was added to bring total volume added to 100%. The solution was mixed well and precipitated at 4° C. for 30 min. After incubation, the sample was centrifuged at 4° C., 10,000 RPM for 20 minutes. The supernatant was removed and the pellet washed with 80% ethanol. The pellet was centrifuged at 10,000 RPM at 4° C. for 10 minutes. The pellet was allowed to dry and dissolved in water. Concentration of tRNA was determined by O.D. (A260/A280; 1 A260 unit=16 μg tRNA) and by 8% TBE-Urea gel analysis.

1.C.3. Isolation and Purification of tRNA Molecules

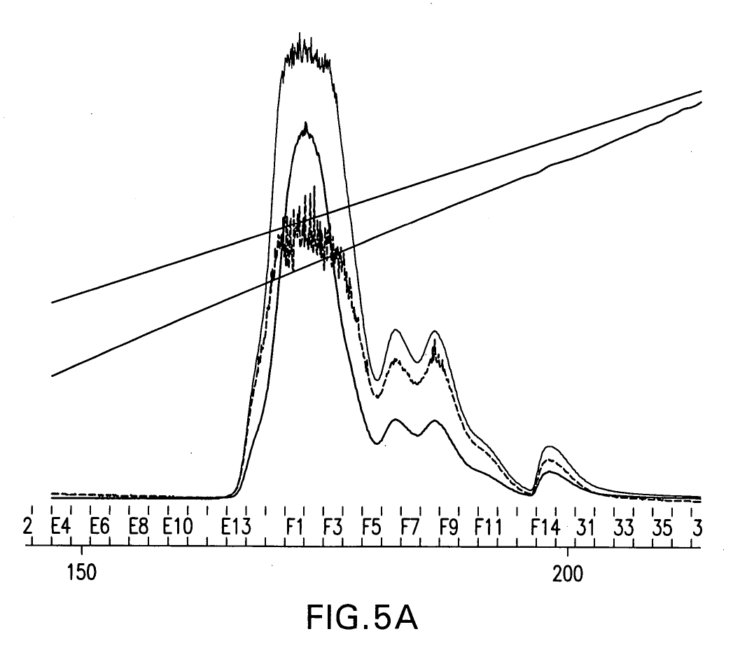

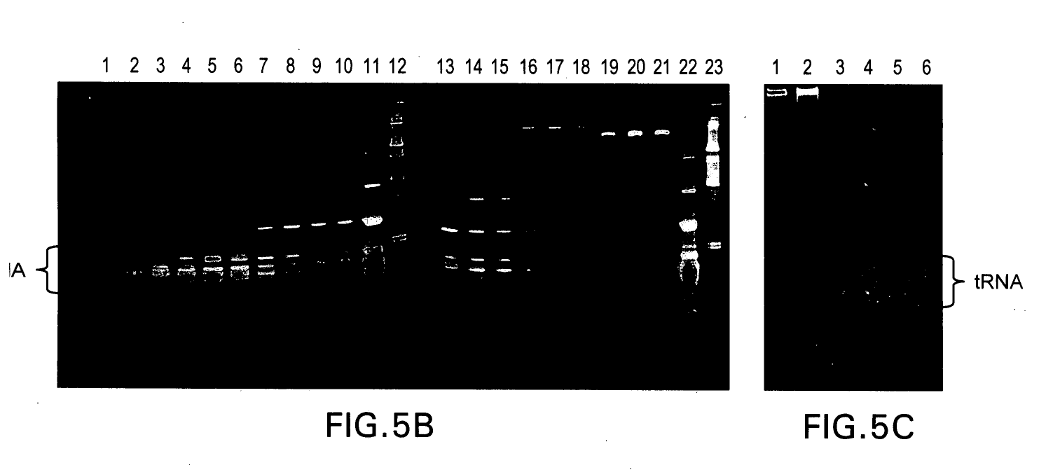

Several methods of lysing the bacterial cells were used before lysates were placed over the Q sepharose column. The starting material before lysis was generally 2-5 g of cell pellet. The one that provided the best yield and gave the least amount of contaminants off the Q-sepharose column was a method, which used a combination of lysozyme, lysis buffer and a polytron to break the cells (see methods and attached protocol). FIG. 5A shows a representative chromatograph of a tRNA lysate resolved from a 10 ml Q sepharose column using a salt gradient. The first major peak, which elutes midway through the gradient, contains the majority of the tRNA (Lanes 2-10, FIG. 5B). The peaks following the tRNA peak contain higher molecular weight RNAs and genomic DNA (Lanes 12-20, FIG. 5B). Final analysis of the purified tRNA shows that the in-house method of purification produces tRNA that has fewer upper and lower molecular weight contaminants than tRNA obtained from Roche (FIG. 5C). Total yield of tRNA from 2-5 g of induced BL21 Star™ induced cells was 5-10 mg of tRNA.

### Example 2

### Evaluation of rctRNA

In order to evaluate the effect of supplementation of the new tRNAs, the following experiments were carried out.

2.A. Linear Expressway Reactions

2.A.1. Materials and Methods

Plasmids—The following plasmids were used for testing expression and activity: pEXPI- Lac Z-DEST (current Expressway™ Plus control, Invitrogen, Carlsbad), Linearized GFP (from pCR2.1 GFP plasmid), and pET21a SsoSSB. All plasmids were prepared using commercial miniprep kits and resuspended in Molecular Biology Grade Water.

2.5× In vitro Protein Synthesis (IVPS) Reaction Buffers—The 2.5× IVPS E. coli Plus reaction buffer minus tRNA was obtained from Invitrogen Corporation, Carlsbad, Calif. (and is a component of the Expressway™ Plus system, available from Invitrogen; Cat. no. 470101).

Protein Synthesis Reactions—For standard Expressway™ Plus—tRNA protein synthesis reactions, 4 μl Dnase/Rnase-free water, 20 μl 2.5× IVPS E. coli Plus Reaction Buffer minus tRNA, 1 μl T7 RNA polymerase mix and 20 μl of IVPS E. coli extract were premixed in 2-ml tubes on ice. Plasmid DNA templates were added at 1 mg and the final volume of the reaction was brought to 50 μl. Reactions were incubated at 37° C. for 2 hours in a thermomixer. After incubation, 5 μl of RNase A (1 mg/ml) was added and reactions were incubated 15 more minutes. Reactions were placed on ice during analysis. For experiments adding either Roche or rare codon tRNAs, reactions were completed as above except the indicated concentrations of Roche or rare tRNA were added to the Expressway™ Plus and Expressway™ Linear S30 Extracts.

TCA Precipitation/Calculation of Yield. Radiolabeled proteins were synthesized by the addition of 0.5 μl of ³⁵S-methionine (3,000 Ci/mmol) to the 50 μl protein synthesis reactions. Total counts were determined by spotting 5 μl of the 50 μl reactions on an individual glass filters and counted directly. Precipitable counts were determined by placing 5 μl of RNase A treated protein synthesis reactions into glass tubes, adding 10% TCA solution and incubating the tubes at 4° C. for 20 minutes. After incubation, the precipitated proteins were passed over glass filters (grade 34 glass fiber) in a filtering apparatus, washed with, 5% TCA, rinsed with 100% ethanol and counted in the scintillation counter. Yield was determined using the following formulas outlined in the Expressway™ Plus manual.

2.B. Expressway™ Linear Reactions

2.B.1. BL21 Star™ tRNA

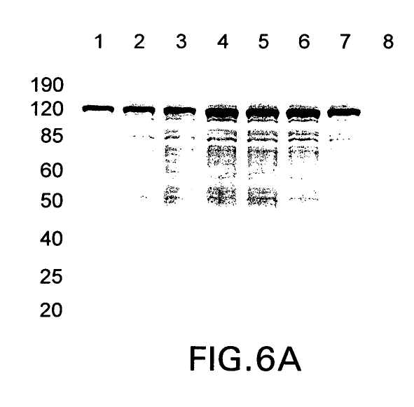

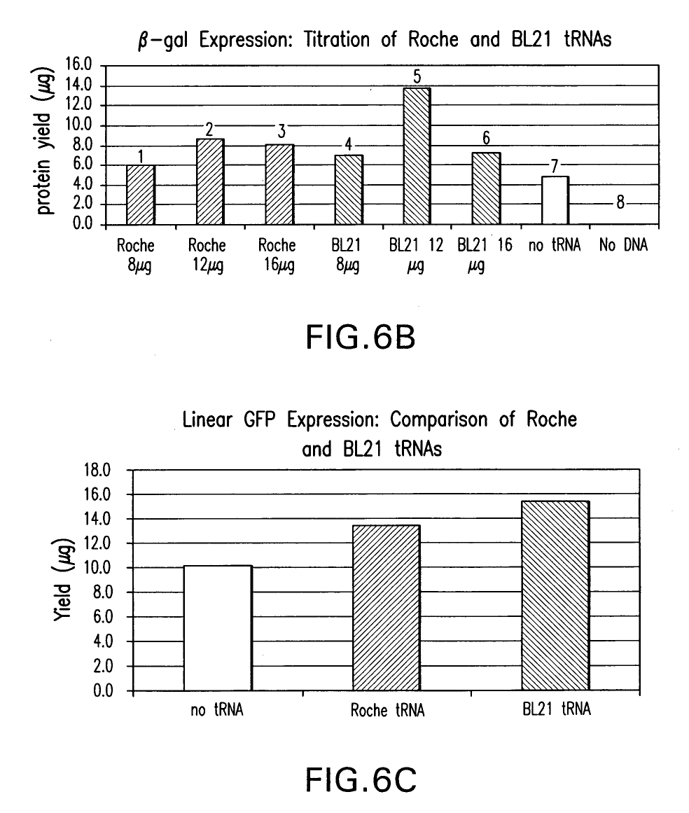

One of the first experiments was to add the in-house purified tRNA to ExpresswayTm Linear reactions. In FIG. 6A, the autoradiograph of beta-gal protein expressed from Expressway™ Linear reactions containing either 8, 12, or 16 mg of Roche tRNA, 8-16 μg of BL21 Star™ in-house purified tRNAs or a reaction without tRNA supplementation shows that the in-house purified tRNA increases protein expression compared to Roche tRNA. The highest yield of beta-gal, as determined by TCA counts, is seen when 12 μg of in-house purified tRNA is added (FIG. 6B). Addition of in-house purified tRNA also increased yield of linear GFP compared to Roche tRNA (FIG. 6C). In general, supplementation of tRNA to the in vitro reactions increases protein yield 30-60% (based on minus tRNA controls).

2.B.2. Expression Using pACYCtRNA(6) tRNA

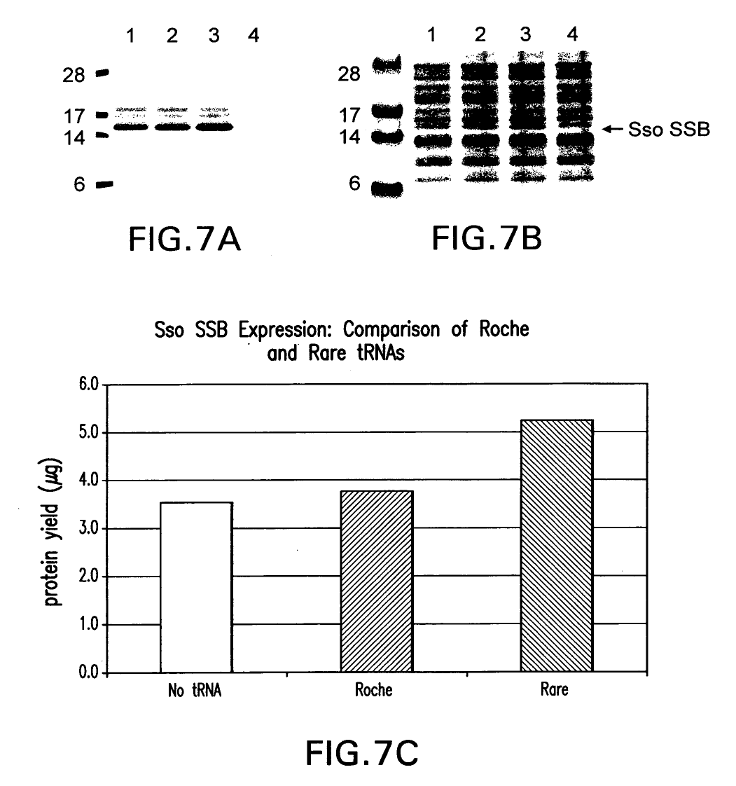

In analyzing the pACYCtRNA(6) tRNA a “rare-codon-protein” was used. Sulfolobus solfataricus single-stranded binding protein (SsoSSB) is a novel crenarchael single-stranded DNA binding protein, which contains AGA, AGG for arginine; ATA for isoleucine; and CTA for leucine, many in pairs or triplets (Haseltine and Kowalczykoski, Mol. Microbiol. 43:1505-1515, 2002). Expression of SsoSSB was compared after the addition of Roche and pACYCtRNA(6) tRNAs in FIG. 7. The pACYCtRNA(6) tRNA increases expression of SsoSSB protein about 40% compared to the Roche tRNA, which does not significantly enhance expression (FIG. 7C). Increased SsoSSB expression after the addition of pACYCtRNA(6) tRNA can clearly be seen in both the autoradiograph and Coomassie-stained gels from these reactions (FIGS. 7A and 7B).

### Example 3

### Preparation of stRNA Via PCR (“PCR stRNA”)

A bacteriophage T4 suppressor tRNA made from the phage T4 psul gene (McClain et al., J. Mol. Biol. 81:157, 1973) was used. This suppressor tRNA is an amber suppressor, which inserts a serine at the UAG codon and is naturally aminoacylated in E. coli (Deutscher et al., J. Biol. Chem. 249:6696, 1974).

The T4 psul gene was amplified from overlapping primers, and the amplified product was used to transcribe in vitro T4 suppressor tRNA. Large-scale transcription reactions of T4 suppressor tRNA were gel-purified or HPLC purified and added to the expression reactions.

3.A. Primers

The phage T4 psul gene was generated using the following overlapping primers:

T7T4tRNA fwd:

(SEQ ID NO:1)

GGATCCTAATACGACTCACTATAGGAGGCGTGGCAGAGTGGTT
and
T4tRNA rev:

(SEQ ID NO:2)

TGGCGGAGGCGATAGGATTTGAACCTATGAGTCGCCGGAGCGACTGCCGG
TTTTAGAGACCGGTG.

3.B. PCR

For PCR, 125ng of each primer was used in a 50 μl reaction containing 1× Platinum® Taq DNA Polymerase High Fidelity buffer (Invitrogen), 200 μM dNTPs, 3 mM MgCl₂, 5 units Platinum® Taq DNA Polymerase High Fidelity (Invitrogen) and water. Products were made using the following cycling conditions: 20 cycles of 95° C., 30 secs; 55° C., 30 secs; 68° C., 2 min. Products were verified for size on a 1% agarose gel and optical densities (O.D. A₂₆₀/A₂₈₀) were measured by spectrophotometer.

### Example 4

### Preparation of Cloned Suppressor tRNA (“cstRNA” and “Total stRNA”)

4.A. Cloning and Expression of stRNA (cstRNA)

A PCR-amplified DNA fragment of the phage T4 psul gene was Topo® cloned into pTrcHis 2A. The cloned T4 psul gene was excised from the pTrcHis 2A plasmid at the HindIII and SphI sites and ligated into a HindIII and SphI digested pACYC184 backbone vector, thus replacing the tet resistance gene in the pACYC184 vector (it retains the camR geneAl9). E. coli cells were transformed with pACYCsupT4 tRNA plasmid, and the cells were plated on LB plates containing chloramphenicol in order to isolate cells containing the pACYCsupT4 tRNA plasmid.

4.B. Purification of Total RNA

4.B.1. Lysis and Cell Pellet Preparations of A19 E. coli Harboring pACYCsupT4 were carried out essentially as described in the preceding Examples.

4.B.2. Chromatography

Cell pellets were dissolved in 10 mM tris pH 8.0, 1 mM EDTA (TE), 250 mM NaCl pH 8. Using FPLC (Amersham ÄKTA™ 10 purifier), a salt gradient was set-up on a 10 ml Q Sepharose starting from 25% TE, 250 mM NaCl to a final concentration of 100% TE, 1M NaCl. The sample was loaded, and one-milliliter fractions were collected at 4° C. Pool fractions were determined by running 5 μl of collected fractions (chosen according to chart peaks) on 10% TBE-Urea Novex gels. Gels were stained in 0.5 ug/ml EtBr+1×TBE for 5 min. Fractions that did not contain genomic DNA and large RNA were pooled.

After fractions were pooled, 1/10 volume of 3M NaOAC pH 4.5 and 0.4 volume isopropanol was added. The solution was mixed well and placed at 4° C. overnight. The mixture was centrifuged at 10,000 RPM at 4° C. for 30 minutes. The supernatant containing the tRNA was collected to a new tube (or bottle). Isopropanol was added to bring total volume added to 100% volume. The solution was mixed well and precipitated at 4° C. for 30 min. After incubation, the sample was centrifuged at 4° C., 10,000 RPM for 20 minutes. The pellet was washed with 80% ethanol and centrifuged at 10,000 RPM at 4° C. for 10 minutes. After air-drying, the pellet was dissolved in water. The concentration of tRNA was determined by O.D. (A₂₆₀/A₂₈₀; 1 A₂₆₀ unit=16 μg tRNA) and by 10% TBE-Urea gel analysis.

This preparation, referred to herein as “Total stRNA”, is a heterogeneous mixture of overexpressed cstRNA and E. coli tRNAs, with the former being present in greater amounts than the latter.

### Example 5

### Preparation of stRNA Transcribed in Vitro (“IVT stRNA”)

5.A. In Vitro Transcription of Suppressor T4 tRNA

5.A. Annealed Oligonucleotides

Two oligonucletoides were used for in vitro transcription (IVT):

T7 forward primer T7Fwd:

| | |
|---|---|
| GGATCCTAATACGACTCACTATATATAGG; | (SEQ ID NO:3) |

and

Rev-T7T4tRNA, consisting of the reverse complement of the complete psui gene (underlined) and a portion of the reverse T7 primer complement:

(SEQ ID NO:4)

TGGCGGAGGCGATAGGATTTGAACCTATGAGTCGCCGGAGCGACTGCCGG
TTTTAGAGACCGGTGCATTAAACCACTCTGCCACGCCTCCTATAGTGAGT
CGTATTAGGATCC.

Twenty-five mM dilutions of two oligonucleotides were mixed in a 1:1 ratio in a microfuge tube. The mixture was incubated at 95 degrees for 5 minutes. The tube was removed and allowed to cool slowly to room temperature (˜20 min). One μl of annealed oligos was used per 40 μl BLOCK-iT™ (Invitrogen) transcription reaction.

5.B. In Vitro Transcription

The annealed oligos, or PCR products generated therewith, were used in 10× scaled-up BLOCK-iT™ (Invitrogen) transcription reactions that were carried out essentially according to the manufacturer's instructions. The 400 μl reactions contained 15 mM rNTPs, 1× BLOCK-iT™ Transcription Buffer, 60 μl BLOCK-iT™ T7 Enzyme Mix, water and 10 μg of suppressor T4 PCR product or 10 μl of annealed oligonucleotides. Reactions were incubated for two hours at 37° C. No DNAse I treatment was performed. Products were verified on 4% agarose gels before further purification.

### Example 6

### Evaluation of Purification Procedures

6.A. Purification Procedures

Suppressor T4 tRNA(stRNA) was produced and purified by a variety of procedures to determine the best and most efficient method for producing preparative quantities of actively suppressing stRNA. Methods used to prepare stRNA included stRNA purified from an E. coli strain A19/pACYCsupT4 (Total stRNA) and in vitro transcribed stRNA (IVT stRNA) purified over a Q sepharose column, and gel-purified IVT stRNA.

6.A.1. “LiCl₂ stRNA”

A portion of the transcription reaction was precipitated with 1/10 volume 7M LiCl₂ and 1 volume isopropanol to generate a LiCl₂ stRNA preparation.

6.A.2. “Gel Pur. IVT stRNA”

Suppressor tRNA from transcription reactions was loaded directly on 10% TBE-Urea Novex gels. Bands corresponding to the suppressor T4 tRNA were extracted using UV-shadowing and eluted from the gel slices in an elution buffer (0.5M NH₄OAC, 10 mM Mg(OAC)₂, 1 mM EDTA and 0.1% SDS) overnight at 4° C. Eluted IVT stRNA was precipitated with 1/10 volume of 3M NaOAC pH 4.5 and 1 volume of isopropanol, incubated at −80° C. for 15 min or −20° C. for ≧30 min and centrifuged at 10,000 RPM at 4° C. for 30 minutes. The pellets were washed with 80% ethanol. After drying, the pellet was dissolved in water. The concentration of tRNA was determined by O.D. (A₂₆₀/A₂₈₀; 1 A₂₆₀ unit=16 μg tRNA) and by 10% TBE-Urea gel analysis.

6.A.3. “FPLC IVT stRNA”

For chromatographic purification of IVT stRNA, transcription reactions were FPLC purified essentially as described above with the following modifications: pooled fractions were precipitated with 1/10 volume of 3M NaOAC pH 4.5 and 1 volume of isopropanol, incubated at −80° C. for 15 min or −20° C. for >30 min and centrifuged at 10,000 RPM (Sorvall SS-34 rotor) at 4° C. for 30 minutes. IVT stRNA pellets were processed essentially the same as described above for.

6.B. Evaluation of Purification Procedures

6.B.1. Content

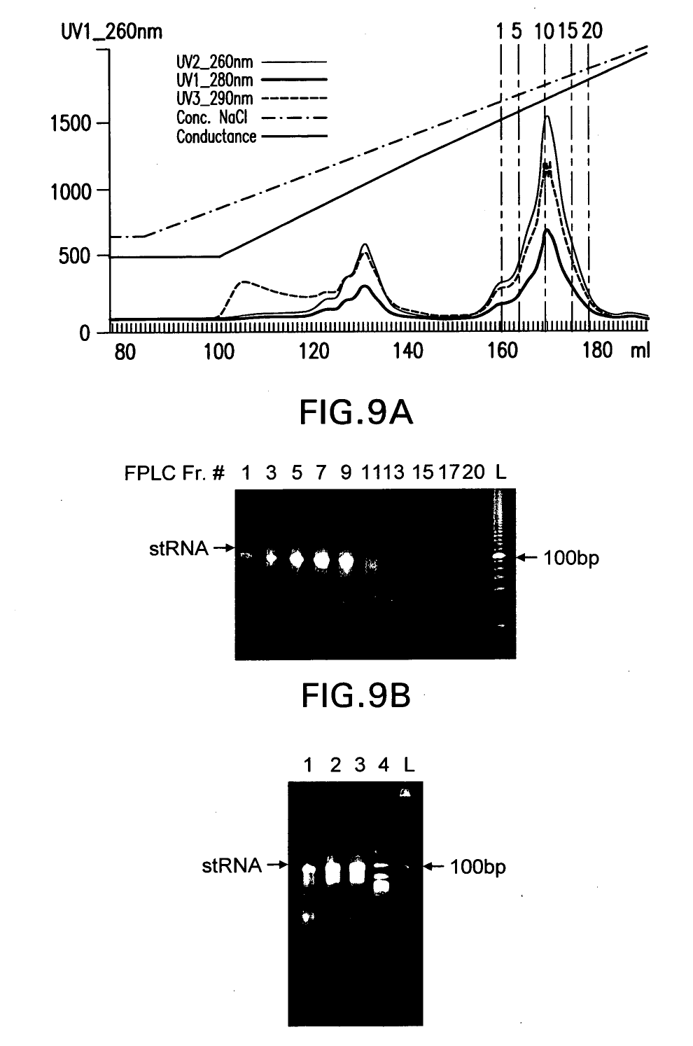

FIG. 9A shows a representative chromatograph of an IVT stRNA preparation fractionated on a Q column using a 25% to a 100% NaCl gradient. Fractions 1-20 were analyzed on a 10% TBE-Urea gel (FIG. 9B). The major band in FPLC fractions 3-9 corresponds to the IVT stRNA (FIG. 9B). Pooled fractions (1-10) were precipitated and compared to equal amounts (based on O.D. readings) of other preparations of stRNA on a 10% TBE-Urea gel (FIG. 9C).

The gel shown in FIG. 9C shows that the four procedures used to purify the stRNA all produce a major band of approximately 100 base pairs. While FPLC fractionation eliminates a majority of the non-stRNA products in the IVT stRNA preparation (compare FPLC IVT stRNA, Lane 1, to LiCl₂ IVT stRNA, Lane 2, in FIG. 9C), the cleanest preparation appears to be the gel purified IVT stRNA (FIG. 9C, Lane 2). The Total stRNA contains not only cstRNA but also total E. coli tRNAs (Lane 4, FIG. 9C). (Although applicants do not want to be bound by any particular theory, the low molecular weight molecules, which are present as faster-migrating material, in lanes 1 and 3 in FIG. 9C may come from incomplete transcripts from the transcription reaction.

6.B.2. Yield

Overall yields from these preparations varied. The final yields from each preparation are as follows: FPLC IVT stRNA prep, 440 μg from a 400 μl transcription reaction; Gel-purified IVT stRNA, 192 μg from a 400 μl transcription reaction; and FPLC Total stRNA, 7200 μg from 2 liters of E. coli cells.

### Example 7

### Titration of Suppressor tRNA Preparations

The suppressor activity of three of the stRNA preparations (FPLC IVT stRNA, Gel-Pur. IVT stRNA and Total stRNA) was investigated by titrating these stRNAs using Expressway™ LumioTm reactions (Invitrogen Corporation; Carlsbad, CA). The DNA construct used to determine the suppression effect of the stRNA additions was pEXP4-SCK (a construct coding for a human kinase ORF similar to creatine kinase), which contains a TAG stop codon at the C-terminus. In the experiments, 0.5 ug, 1 ug, 5 ug, 10 ug, 15 μg or 20 μg of each stRNA was added to the Expressway™ Plus Lumio™ reactions.

For protein synthesis reactions, 20 μl 2.5×IVPS Plus E. coli reaction buffer, 20 μl of IVPS E. coli extract (A19 slyD::kan), 1 μl T7 RNA polymerase mix, and 1 μl of 75 mM methionine were premixed on ice. To the mixture, 0.5 μg, 1 g, 5 μg, 10 μg, 15 μg or 20 μg of each stRNA (FPLC, GEL-P, cstRNA) was added. One microgram of DNA was added and the final volume of the reaction was brought up to 50 μl with water.

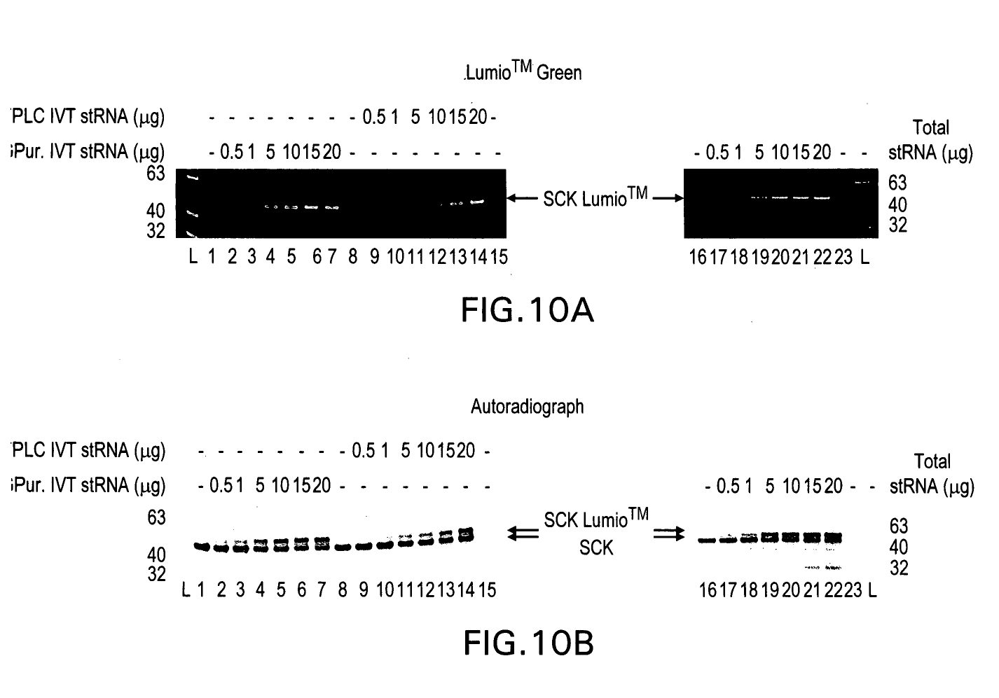

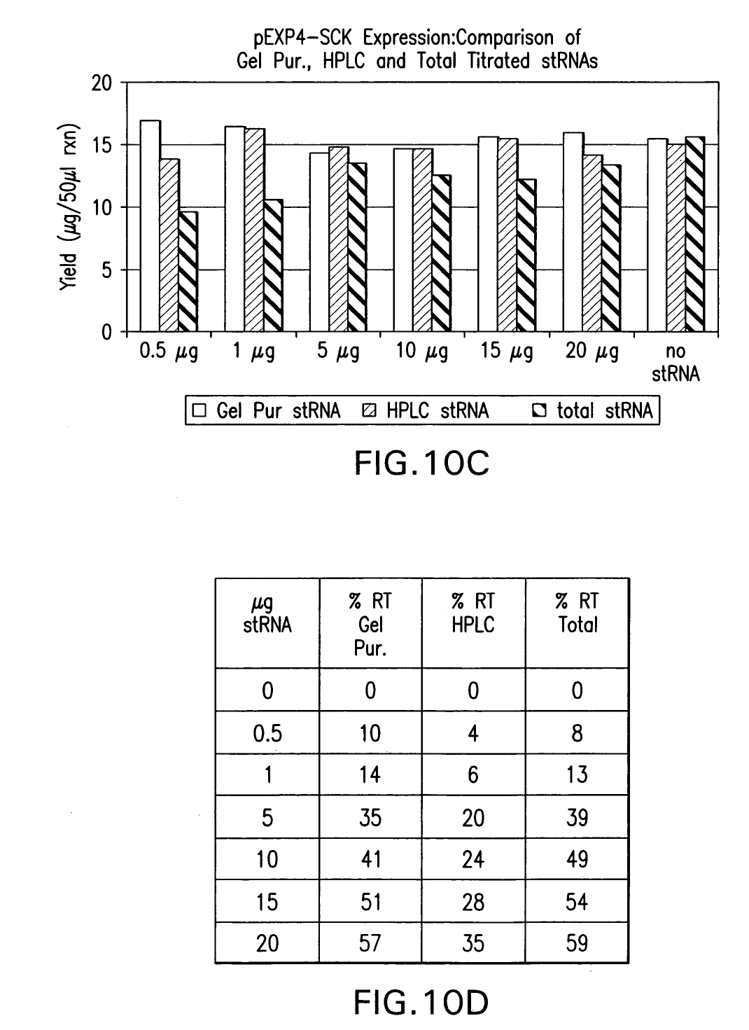

The results show that the read-through at the UAG codon of the ‘similar to creatine kinase’ human ORF increases as concentrations of stRNA increase (FIGS. 10A, 10B and 10C). FIG. 10A illustrates this increase by the ability to detect increasing amounts of the Lumio™ fusion proteins by the Lumio™ Green Reagent.

A difference between Lumio™-tagged SCK and native SCK is visible when ³⁵S-methionine was used in the synthesis reactions (FIG. 10B). Using Phosphorimage analysis, the percent read-through for each stRNA was determined and compared in FIGS. 10C and 10D. Based on this analysis the activity of both the Gel-Pur. IVT stRNA and the Total stRNA are comparable. The reduced activity of the FPLC IVT stRNA prep is most likely due to the dilution of the full-length stRNA as result of fraction pooling.

Unlike either of the IVT stRNAs, addition of Total stRNA increases the yield of SCK at higher concentrations (FIG. 10C), but this may be due in part to the heterogeneous mixture of overexpressed cstRNA and E. coli tRNAs present in Total stRNA.

### Example 8

### A Gateway® Destination Vector Containing a C-Terminal Lumio™ Tag

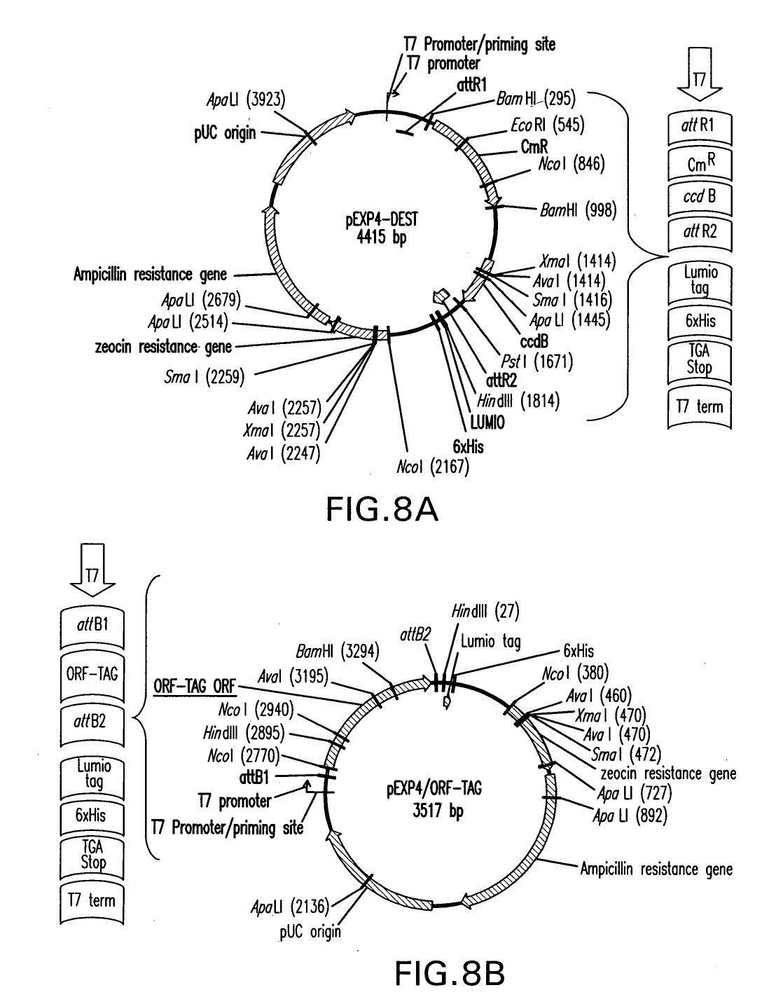

A Gateway® DEST destination vector containing a C-terminal Lumio™ tag was designed, constructed and designated pEXP4-DEST. The plasmid pEXP4-DEST contains LumioTm and 6×His tags followed by a TGA stop codon, which are positioned downstream from attR2, a site-specific recombination sequence (see FIG. 8). Ordinarily, proteins from genes cloned into the pEXP4-DEST vector will be expressed in their native wildtype form. However, if the gene of interest contains a TAG stop codon and the appropriate suppressor tRNA is added to the reaction, a 6×His-Lumio™ fusion will be synthesized. The pEXP4-DEST vector is compatible with the C-terminal labeling of proteins expressed using clones from the Ultimate™ ORF collection (Invitrogen), in which all the clones contain a UAG stop codon, and provides high levels of expression in the LumiOTM S30 extract.

8.A. Construction of pEXP4 Lumio™ Tag Destination Vector

The pEXP4-DEST vector was made by replacing the existing C-terminal cassette in the pEXP2-DEST™ vector (Invitrogen) with the C-terminal cassette from the pET161-DEST (a.k.a. pETDEST42/FlAsH) (Invitrogen) vector (FIG. 8A). Both vectors were digested with BIpI and PstI, and the backbone of the pEXP2-DES™ vector was ligated with the insert from the pET161-DEST vector. Library Efficiency DB3.1 Competent Cells (Invitrogen) were transformed by the ligation mixtures, and the cells were plated on LB plates containing chloramphenicol.

Colonies were screened by DNA miniprep and positive clones were sequenced. The sequence for the pEXP4-DEST vector is provided in the Sequence Listing (SEQ ID NO:5).

8.B. In Vitro Site-Specific Recombination of pENTR-Clone and pEXP4-Destination Vector Using Gateway®Technology pENTR-Clones.

The following Human ORFs Kinases were successfully cloned into the pEXP4 Lumio™ Tag Destination Vector using LR Clonase™ mix with the following clones, which are provided in a Gateway® entry 007462; vector: Creatine kinase B (Genbank #NM_{—}01823, gi: 34335231; Invitrogen catalog number 10H5211); a kinase similar to creatine kinase from muscle (Genbank #BC007462, gi:13938618; IOH7287); a cAMP dependent protein kinase (Genbank#BC016285; gi:16740847; Invitrogen catalog #IOH10103); a serine/threonine protein kinase (Genbank #NM_{—}032037, gi:51477706; IOH10991); and casein kinase epsilon 1 (Genbank# NM_{—}001894; gi:40549399 Invitrogen catalog number 1OH21160).

After incubating for one hour, the mixture was used to transform chemically competent E. coli DH5α cells (Invitrogen). The transformed cells were plated, and colonies picked for DNA isolation. The in vitro recombination reactions were carried out essentially according to the manufacturer's (Invitrogen) protocol. The five resulting vectors, each containing a different human kinase ORF, were used in subsequent Examples.

### Example 9

### ANTI-RF1 Antibody

Another component for an IVPS system of the invention is an antibody that recognizes a translation termination factor, for example the E. coli Release Factor 1 (RF1). RF1 acts in the termination of translation at the amber (UAG) and ochre (UAA) codons (Craigen et al., Mol. Microbiol. 4:861, 1990). A number of laboratories have shown that depletion of RF1 increases the read-through at the amber and ochre stop codons (Shimizu et al., Nat. Biotechnol. 19:751, 2001; Short et al., Biochemistry 38:8808, 1999).

9.A. RF1 Fusion Protein

The RF1-encoding prfl (a.k.a. prfA) gene from E. coli was PCR-amplified and cloned in the vector pRSET-A (Invitrogen). The resulting plasmid pFKI005 (SEQ ID NO:6) encodes a fused protein consisting of RF1 fused at its C-terminal to a TEV cleavage site followed by a 7×His Tag.

9.B. Immunization

The protein was expressed and purified over a nickel-chelating column (Amersham Biosciences). Forty mg of RF1-TEV-7×His (>90% pure) were obtained from 1 liter of cell culture. A fraction of the sample was digested with the TEV Protease (Invitrogen) and was again run over a nickel-chelating column. Three mg of the eluate (>99% pure cleaved protein) was used as an immunogen for antibody production in rabbits (EvoQuest, Invitrogen).

9.C. Purification

9.C.1. Antigen Based Affinity

RF1 antibody was affinity purified by EvoQuest™ using an RF1-TEV-7×His affinity column.

9.C.2. Protein A Affinity

Anti-RF1 antibody was also purified as a population of IgGs using a protein A sepharose column.

9.C.2.a. Crude Sera Preparation

This procedure used 10 ml of sera from rabbit B5142 (4-21-04), B5142E112B (Evoquest project H0320801I). The 10 ml serum was brought to a 50 mM sodium borate concentration by adding 0.5 ml 1 M sodium borate. The pH was checked at 8.5.

9.C.2.b. Buffers:

1M Sodium Borate Stock Solution is made by preparing 1 M boric Acid (F.W. 61.83g) and adjusting the pH with 10 N NaOH. The solution is filter sterilized after preparation.

Column Buffer A: 50 mM Sodium Borate, pH 9, 5% glycerol. For 1 Liter—12.5 ml 1 M sodium borate stock solution, pH 9, 15.5 ml 80% Glycerol, bring up to volume with sterile water after adjusting the pH to 9.0 with 10 NaOH. The solution is filter sterilized after preparation.

Column Buffer B: 100 mM Glycine pH 3. The solution is filter sterilized after preparation.

9.C.2.c. Column Preparation

For this procedure a 1 ml Protein A sepharose column was used for 10 ml of crude sera. A larger volume column can be used for larger volumes of crude sera (e.g., 25 ml column for 150-200 ml of crude sera). The Protein A sepharose was Amersham's nProtein A sepharose 4 Fast flow (17-6002-35). The column was poured and equilibrated in Column Buffer A.

9.C.2.d. FPLC procedure.

For this preparation an AKTA 10 purifier was used with the manual settings. However, a program procedure can be written using the information below:

Equilibrated the 1 ml column with 5 column volumes Column Buffer A at 1 ml/min

10 ml of buffered sample was loaded with a superloop after the column was equilibrated. The sample was loaded at 1 ml/min. The flowthrough was collected in 4 ml fractions.

Washed the column with 10 column volumes of Column Buffer A at 1 ml/min or until the flowthrough peak returned to baseline.

Eluted the sample with 7 column volumes of Column Buffer B collecting 0.5 ml fractions. NOTE: The eluate was collected in 12 mm tubes containing 20 μl of 1 M Tris base pH11.1 to re-equilibrate the pH of the IgGs/antibody after the elution with glycine. Before starting the column procedure, test the pH of 0.5 ml of Column buffer B with 20 μl of Tris base pH 11.1 to make sure the pH is at ˜8.00.

Re-equilibrated the column with Column Buffer A. Store the column in 1×PBS pH 8.0, 0.01% sodium azide.

9.C.2.e. Dialysis

Peak samples (0.5 ml) were dialyzed separately (e.g. fraction 10 and fraction 11 from the peak above were placed in separate slidalyzers) in Pierce 0.5-3 ml, 3500 MW cut-off Slidalyzers (cat# 66330). The dialysis buffer was 500 ml, lXPBS pH 8.0. The samples were dialyzed for 1 hour in 500 ml 1×PBS, pH 8.0 before changing out the buffer (1×PBS, pH 8.0) and dialyzed another hour. The sample was stored at 4° C.

### Example 10

### Reagents and Procedures for In Vitro Protein Synthesis (IVPS)

10.A. Buffers

10.A.1.S30 Wash Buffer

0.01 M Tris-Oac, pH 8.2; 0.014 M Mg(OAc)2; 0.06 M KCl; and 0.006 M 2-Mercaptoethanol (BME).

10.A.2.S30 Resuspension Buffer

0.01 M Tris-Oac, pH 8.2; 0.014 M Mg(OAc)2; 0.06 M KOAc; 0.001 M DTT; and 0.5 mM PMSF.

10.A.3.Translation Buffer (10×)

0.733 M Tris-Oac; 11 mM DTT; 23 mM Mg(OAc)2; 100 μM Amino Acids; 33 mM ATP; 210 mM PEP/K; AND 1260 U/mL Pyruvate Kinase.

Expressway™ Plus with Lumio™ Technology 2.5×IVPS reaction buffer was used except where a −tRNA 2.5×IVPS buffer (i.e., tRNA omitted) is noted.

10.A.4.Final concentration of components in A 50 μl reaction

TABLE 2
IVPS FINAL CONCENTRATIONS

| Component | Final Concentration | |
|---|---|---|
| HEPES (pH 7.5) | 57 | mM |
| DTT | 1.76 | mM |
| Sodium ATP | 1.2 | mM |
| Sodium CTP | 0.86 | mM |
| Sodium GTP | 0.86 | mM |
| Sodium UTP | 0.86 | mM |
| Folinic Acid | 34 | μg/ml |
| Acetyl Phosphate | 30 | mM |
| Potassium Glutamate | 230 | mM |
| Ammonium Acetate | 80 | mM |
| Magnesium Acetate | 12 | mM |
| Camp | 0.66 | mM |
| PEP/K | 30 | mM |
| PEG-8000 | 2% | |
| T7 RNA Polymerase | 65 | μg/ml |
| S30 Extract | 14 | mg/ml |
| Methionine | 1.5 | mM |
| Tyrosine | 500 | μM |
| Other amino acids | 1.25 | mM |

10.B. S30 Extract

Generally, the procedure for preparing S30 extracts is as follows:

10.B.1. Cell Resuspension

Thaw cells at room temperature for 30 min.

Resuspend each gram of cells in 1 ml of chilled (4° C.) S30 buffer with DTT added immediately prior to use (for example, 250 ml S30 buffer for 250 g cells). Swirl the cells gently by hand for a few minutes (without generating froth) to hasten the resuspension process. Place a sterile stir bar into bottle containing cells and stir gently for approximately 15 min to completely resuspend cells. Place on ice immediately. Do not add any more buffer, as volume is critical to final total protein concentration of extract.

Measure the volume of the suspension. The volume (in ml) should be approximately twice the weight of the starting material (in grams).

Example: If there are 50 g of starting material, the volume of resuspended cells is ˜100 ml.

Filter through a piece of sterile cheesecloth into a sterile 1 L side-arm flask.

Remove 5 ml resuspended cells and place into 995 ml water (1:200 dilution) to determine a starting OD. Vortex this sample and read at 590 nm using water as a blank. Record OD on 54423.PBR.

Attach the side-arm flask containing the cells to a vacuum pump and de-gas cells for approximately 15 min. Swirl cells occasionally to promote degassing. Once cells are degassed, be careful not to swirl or generate bubbles.

10.B.2. Cell Disruption

Use the Emulsiflex C50 homogenizer to disrupt cells. Do not substitute mini-Gaulin for the Emulsiflex.

Homogenizer must be chilled for 1 h before use.

Turn on the compressed air outlet to 115-120 psi, and set timer to 60 min.

Set homogenizing pressure to 25,000 psi.

Adjust the black regulator knob to a reading of 80-85 psi.

Place a sterile 0.5 L container at the outlet receiving reservoir.

Fill the inlet reservoir with the de-gassed and filtered cell suspension.

Press the START button to disrupt the cells. Ensure that the pressure is at 25,000-30,000 psi. The homogenizer may stall if the pressure exceeds 30,000 psi. If so, very slowly lower the regulator gauge in small increments to restart the flow. Never lower the pressure below 25,000 psi. It should take approximately 15-20 min to pass 500 ml cell suspension through the homogenizer.

10.B.3.Determine the Efficiency of Lysis for the First Pass:

Gently swirl the lysate to mix.

Prepare a 1:200 dilution by placing 5 ml lysed cells into 995 ml water.

Read the OD at 590 nm using water as blank.

Calculate efficiency of lysis as follows. Record on 54423.PBR.

(First Pass OD590/initial OD590, see above)×100=% not lysed

100−% not lysed=% efficiency of lysis

Efficiency of lysis should be greater than 90%. If less then 90%, pass the cell suspension through the homogenizer again.

Immediately add 1 M DTT to lysate to a final concentration of 1 mM (e.g., 250 ml 1 M DTT per 250 ml lysate).

Centrifuge at 16,000 rpm (30,000×g) in the SS34 rotor for 40 min at 4° C. Do not exceed 35 ml per SS34 centrifuge tube (depending on volume, more than one SS34 rotor may be needed).

During centrifugation, prepare 75 ml pre-incubation mix: 5× Pre-incubation Mix, Final concentration: 0.44 M Tris, pH 8.2 at 22C, 13.8 mM magnesium acetate, 20 mM ATP, pH 7, 126 mM phophoenol pyruvate (PEP), 60 micromolar amino acid mix (-met); 60 micromolar methionine, 10.08 units per mL pyruvate kinase (PK)

Remove the upper four-fifths of supematant with a sterile plastic graduated pipet and collect in a sterile 1 L container. Be careful to not pour off the supernatant because the pellet is very loose.

Measure the volume of supematant and record on 54423.PBR. The volume (in ml) will be approximately the same as the weight of starting material (Example: For 50 g cells, the volume of supernatant is ˜50 ml).

Add 5 ml pre-incubation mix per 25 ml supernatant (Example: 250 ml supernatant will require 50 ml pre-incubation mix).

Incubate in a 37° C. shaking water bath, shaking gently 150 rpm for 80 min. Do not allow the solution to shake enough to form bubbles

10.B.4. Dialysis

Dialyze 3×45 min with 50 volumes of S30 buffer (containing DTT) at 4° C. (For example: 250 ml lysate is dialyzed in 12.5 L S30 buffer per change). Use ¾-inch dialysis tubing with a molecular weight exclusion limit of 12,000 to 14,000 daltons. Rinse well with distilled water prior to use.

Pour dialyzed material into sterile, dedicated SS-34 centrifuge tubes.

Centrifuge at 4,000 rpm (3000×g) with the SS-34 and rotor for 12 min at 4° C. Remove supernatant using a sterile plastic graduated pipet. Do not pour off the supernatant because the pellet is very loose. Immediately place on ice.

Measure the volume of supernatant and record.

Mix the supernatant well by gently swirling and remove one 200 ml aliquot for Bradford analysis of total protein concentration.

Distribute in 25 ml aliquots in 50 ml conical tubes.

Freeze all aliquots in liquid nitrogen using a Cyromed.

Alternatively, freeze aliquots by submerging in dry ice for 30 min.

Remove samples from Cryomed into dry ice bucket and transport immediately to storage at −80° C.

10.B.5. Protein Determination

The following day, thaw an aliquot and perform a Bradford assay. Total protein should be 28 to 42 mg/ml.

10.B.6.Variations

The S30 extract from Expressway™ Plus with Lumio™ Technology was used. Briefly, E. coli IVPS S30 extract was made according to the preceding protocol with the following changes. E. coli IVPS S30 Extract was made from the A19 slyD::kan strain. The A19 slyD::kan strain requires 50 mg/ml kanamycin antibiotic during 6-8 hour and overnight growth. Note that the antibiotic is not required during the fermentation. The cell pellet was resuspended in S30 Buffer containing 0.5 mM PMSF before lysing. The preincubation time was changed to 150 minutes. Before aliquoting the S30 a fmal concentration of 0.425 g/ml E. coli tRNA was added (0.17mg/ml final concentration of total tRNA in a 50 μl reaction).

10.C. Amino Acid Mixes

Amino acid mixtures were prepared according to the following procedure. It is critical that all of the amino acid components are included in the final amino acid mix. The final mix will contain a final concentration of 50 mM for each component.

All amino acids used in the preparation were ordered as a single unit of powdered material from Sigma. Amino acids were added in the order written in Table 3, below.

TABLE 3
Components of Amino Acid Mixtures

| | | Amount to make |
|---|---|---|
| Amino Acid | M.W (g/mol) | 100 ml of 50 mM mix |
| Alanine | 89.1 | 0.45 g |
| Arginine | 174.2 | 0.87 g |
| Asparagine | 150.1 | 0.75 g |
| Aspartate (Aspartic acid) | 133.1 | 0.67 g |
| Cysteine | 121.2 | 0.61 g |
| Glutamate (Glutamic acid) | 147.1 | 0.74 g |
| Glutamine | 146.1 | 0.73 g |
| Glycine | 75.1 | 0.38 g |
| Histidine | 155.2 | 0.78 g |
| Isoleucine | 131.2 | 0.66 g |
| Leucine | 131.2 | 0.66 g |
| Lysine | 182.7 | 0.91 g |
| Methionine | 149.2 | 0.74 g |
| Phenylalanine | 165.2 | 0.83 g |
| Proline | 115.1 | 0.58 g |
| Serine | 105.1 | 0.53 g |
| Threonine | 119.1 | 0.60 g |
| Tryptophan | 204.2 | 1.00 g |
| Tyrosine | 181.2 | 0.91 g |
| Valine | 117.2 | 0.59 g |

Weigh the first component accurately to +0.01 g. Add weighed powder into an appropriately sized sterile container with a screw cap lid.

Repeat weighing procedure with the next component, and add to the container. Continue until all 20 amino acids are weighed and added to the sterile container. Once all 20 components are combined, add GIBCO water to a final volume of 100 ml.

Place on a Labquake and gently rock to get as much of the mix into solution as possible (˜30 to 120 min at room temperature). The final mix will be a slurry, not a completely dissolved solution.

Aliquot the slurry into sterile 50-ml Falcon tubes at 20 ml/container. It is critical that the slurry be extremely well mixed so that the insoluble components are evenly distributed immediately before aliquotting. Do not aliquot if the slurry has been settling for more than 10 s without stirring.

The mix can be stored at −20° C. for up to 2 years.

10.D. Protein Synthesis Reactions

For protein synthesis reactions, 20 μl 2.5× IVPS Plus E. coli reaction buffer, 20 μl of IVPS E. coli extract (A19 slyD::kan), 1 μl T7 RNA polymerase mix, and 1 μl of 75 mM methionine were premixed on ice. One microgram of DNA was added and the final volume of the reaction was brought up to 50 μl with water. Where indicated, stRNAs (10 μg/50 μl rxn) total tRNA with overexpressed sup T4 tRNA or purified anti-RFl antibody/sup T4suptRNA (8 μg and 10 μg/50 μl rxn) mix was were added where noted. Where indicated, various concentrations of stRNA were titrated into the reactions.

10.E. TCA Precipitation/Calculation of Yield

Radiolabeled proteins were synthesized with the addition of 1 μl of [35S] methionine (1135 Ci/mmol) to the 50 ml protein synthesis reactions. Total counts were determined by spotting 5 μl of the 50 μl reactions on individual glass filters and counting directly. Precipitable counts were determined by placing 5 μl of RNase A treated protein synthesis reactions into glass tubes, adding 10% TCA solution and incubating the tubes at 4° C. for 20 minutes. After incubation, the precipitated proteins were passed over glass filters (grade 34 glass fiber) in a filtering apparatus, washed with 5% TCA, rinsed with 100% ethanol and counted in the scintillation counter. Refer to the Expressway™ plus manual for details about yield calculation.

10.F. SDS Page

Gel samples were prepared by precipitating 5 μl of the 50 μl reactions with 20 μl of 100% acetone and incubating at 4° C. for ˜20 minutes. Reactions were pelleted in a microcentrifuge, aspirated to remove acetone, and resuspended in 20 μl of corresponding 1×SDS sample buffer containing 20 mM Lumio™ Green detection reagent, essentially according to the Lumio™ detection protocol (Invitrogen). The samples were heated at 70° C. and 2 μl of in Gel enhancer was added. Five ml were loaded on to 4-12% Bis/Tris NuPAGE gradient gels.

### Example 11

### Effect of Antibody to RF1 on IVPS

One μl of purified RF1 antibody (8 μg/μl) was added per 20 μl E. coli S30 extract. Antibody and S30 were premixed and frozen at −80° C. before use. A mix of RF1 antibody (8 μg/μl) and stRNA (10 μg/μl in water) was made in a 1:1 ratio.

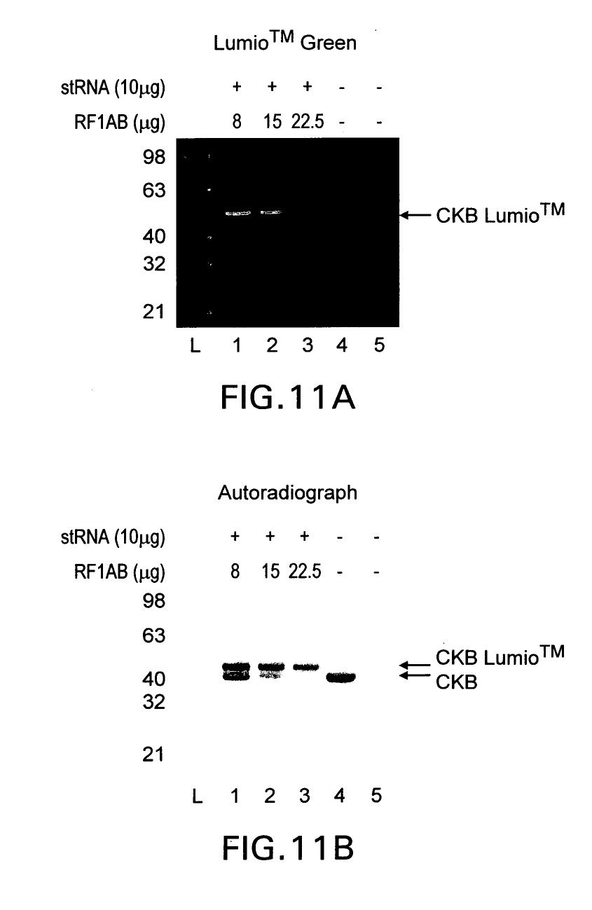

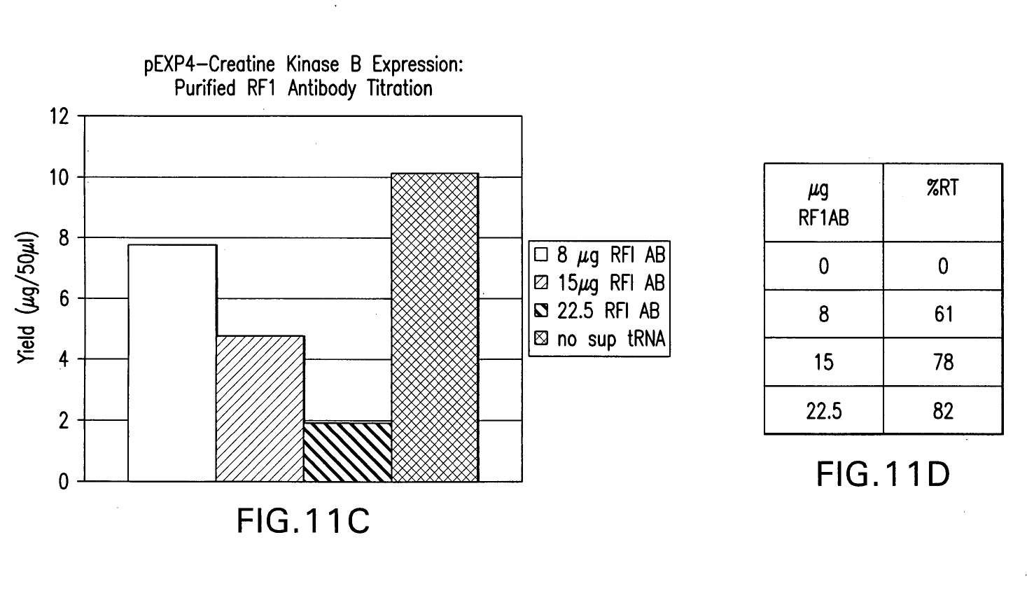

Transcription-translation reactions were set-up using a pEXP4 expression plasmid encoding the human creatine kinase B ORF ending in the UAG stop codon, 10 μg of Total stRNA, and variable amounts of the purified antibody. Addition of the purified RF1 antibody to the expression reactions significantly increases the efficiency of read-through (determined by Phosphorimager analysis comparison of densities between native and LumioTM fusion bands) at the UAG codon (FIGS. 11A, 11B and 11C). The shift from the native creatine kinase B to a Lumio™-fusion protein is clearly seen on the autoradiograph (FIG. 11B). The fact that one fluorescent band is visible by in-gel detection indicates that the gel-shift is due to the addition of the Lumio™ tag (FIGS. 11A and 11B). Although the amount of read-through increased when higher concentrations of antibody were added to the expression reactions, the total yield of protein decreased proportionately (FIG. 11D). Based on these results, 8 μg of RF1 antibody was typically used in other IVPS experiments.

### Example 12

### Expression and Read-Through of the Stop Codons of pEXP4 Human ORF Clones

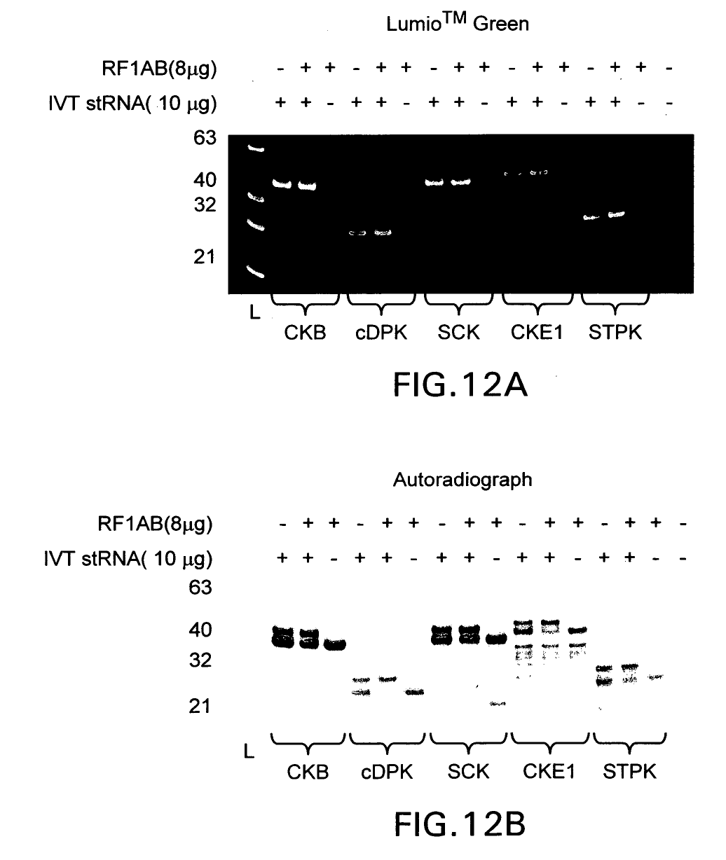

Initial experiments tested the addition of RF1 antibody and stRNA exogenously to the expression reactions. Five different human ORFs, each of which ended in the UAG (amber) stop codon, were expressed in the presence or absence of a mixture of 8 μg of purified RF1 antibody and 10 μg of either Total stRNA or Gel-Pur. IVT stRNA. A representative Lumio™ Green gel and autoradiograph are shown in FIGS. 12A and 12B. The audioradiograph most clearly shows that a higher ratio of Lumio™ tag fusions for each of the five human ORF proteins is seen with the addition of both the stRNA and RF1 antibody compared to addition of the stRNA alone (FIG. 12B). A summary of the read-through efficiencies and protein yields from five experiments are shown in Tables 4 and 5.

TABLE 4
Average Percent Read-Through (R-T) at the UAG Codon for
Five Human ORFs as Determined by Phosphorimagor Analysis

| | | % R-T stRNA | % R-T |
|---|---|---|---|
| | % R-T stRNA | (10 μg) and | RF1 only |
| Human ORF | only (10 μg) | RF1 Ab (8 μg) | (8 μg) |
| Creatine Kinase B (CKB) | 38.3 ± 8.3 | 54.0 ± 5.0 | 0 |
| cAMP-dependent protein | 42.0 ± 5.6 | 67.0 ± 3.4 | 0 |
| kinase (CDPK) | | | |
| Kinase Similar to | 43.0 ± 8.0 | 59.3 ± 5.4 | 0 |
| Creatine Kinase (SCK) | | | |
| Casein Kinase Epsilon 1 | 43.7 ± 8.3 | 65.0 ± 8.2 | 0 |
| (CKE1) | | | |
| Serine/Threonine Protein | 42.7 ± 3.0 | 59.0 ± 5.0 | 15.7 ± 4.0 |
| Kinase (STPK) | | | |

TABLE 5
Average Yield of Five Human ORFs with the
Additions of stRNA and RF1 Antibody as Determined
by ³⁵S-Methionine Incorporation

| | | % R-T stRNA | % R-T |
|---|---|---|---|
| | % R-T stRNA | (10 ug) and | RF1 only |
| Human ORF | only (10 μg) | RF1 Ab (8 μg) | (8 μg) |
| Creatine Kinase B (CKB) | 13.6 ± 3.0 | 14.0 ± 6.4 | 14.8 ± 2.1 |
| cAMP-dependent protein | 10.8 ± 3.8 | 10.9 ± 4.3 | 6.9 ± 0.5 |
| kinase (CDPK) | | | |
| Kinase Similar to | 17.1 ± 4.1 | 15.6 ± 2.2 | 14.2 ± 1.4 |
| Creatine Kinase (SCK) | | | |
| Casein Kinase Epsilon 1 | 11.4 ± 3.1 | 9.8 ± 1.8 | 9.5 ± 0.2 |
| (CKE1) | | | |
| Serine/Threonine | 11.8 ± 2.3 | 10.5 ± 2.9 | 4.3 ± 0.3 |
| Protein Kinase (STPK) | | | |

While addition of the stRNA alone provides similar levels of read-through efficiency for each protein (Table 4, column 1), the level of increase in read-through with addition of both RF1 antibody and stRNA is protein dependent. Differences in read-through efficiencies for each protein may depend on relative expression levels, as there appears to be a correlation between higher yield of protein and lower percentage of read-through (Tables 4 and 5, column 2). The difference in read-through efficiency could also depend on the context of amino acids surrounding the UAG (Namy et al., EMBO Rep. 2:787, 2001). This preference for a particular codon context at the UAG could explain why a small percent of fusion protein is made when RF1 antibody alone is added to the serine/threonine protein kinase expression reaction (Table 4, column 3).

### Example 13

### Effect of Freeze/Thaws on RF1 Antibody Addition

For freeze/thaw reactions, RF1 antibody (8 μg/μl), anti-RF1 antibody/ sup T4suptRNA (8 μg and 10 μg/50 μl rxn) mix and RF1 antibody/IVPS E. coli extract (8 μg/20 μl extract) mix were frozen using liquid nitrogen then thawed for 5 times before adding to reactions. Reactions were incubated at 37° C. for 2 hours in a Thermomixer (Brinkmann) or placed in 96-well plate in a Fluorometer (Costar Black plate). After incubation, reactions that contained ³⁵S-methionine were subjected to RNase A treatment. After incubation, (5 μl of RNase A (lmg/ml) was added (if it was radiolabeled) and reactions were incubated 15 minutes at 37° C.

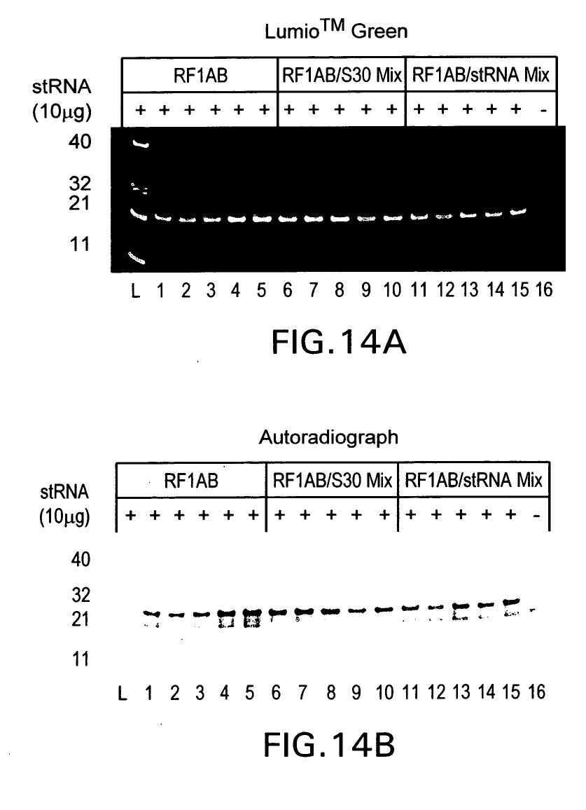

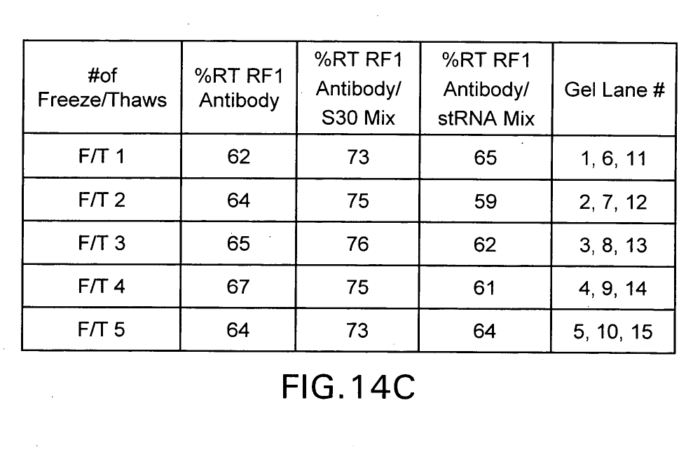

The read-through capabilities of (1) the RF1 antibody alone (with subsequent stRNA addition), (2) the RF1 antibody/suptRNA mixture and (3) an RF1 antibody/S30 extract mixture (with subsequent stRNA addition) were tested after multiple freeze thaws. The results from expression reactions using cAMP Dependent Protein Kinase show that the RF1 antibody most effectively improves read-through when added to the S30 extract (FIGS. 14A and 14B). The efficiency of read-through does not significantly diminish for each type of addition even after 5 freeze/thaws (FIG. 14C).

### Exmaple 14

### Protein Detection

14.A. Real-Time Lumio™ Green Incorporation.

Real Time Labeling of Lumio™ green reagent to the Lumio™-tagged Protein. Real-time incorporation of Lumio™ was measured directly from 50 ml in vitro protein synthesis reactions with 20 mM FlAsH-EDT₂Lumio™ green detection reagent substrate in a 96 well plate at 37° C. In-gel detection of the protein bands was performed using a Typhoon™ 8600 Variable-mode Imager (Molecular Dynamics) equipped with a green 532 nm laser and a fluorescein 526 SP emission filter. Readings were collected at 5-minute intervals over a 2-hour incubation period. A UV box may also be used.

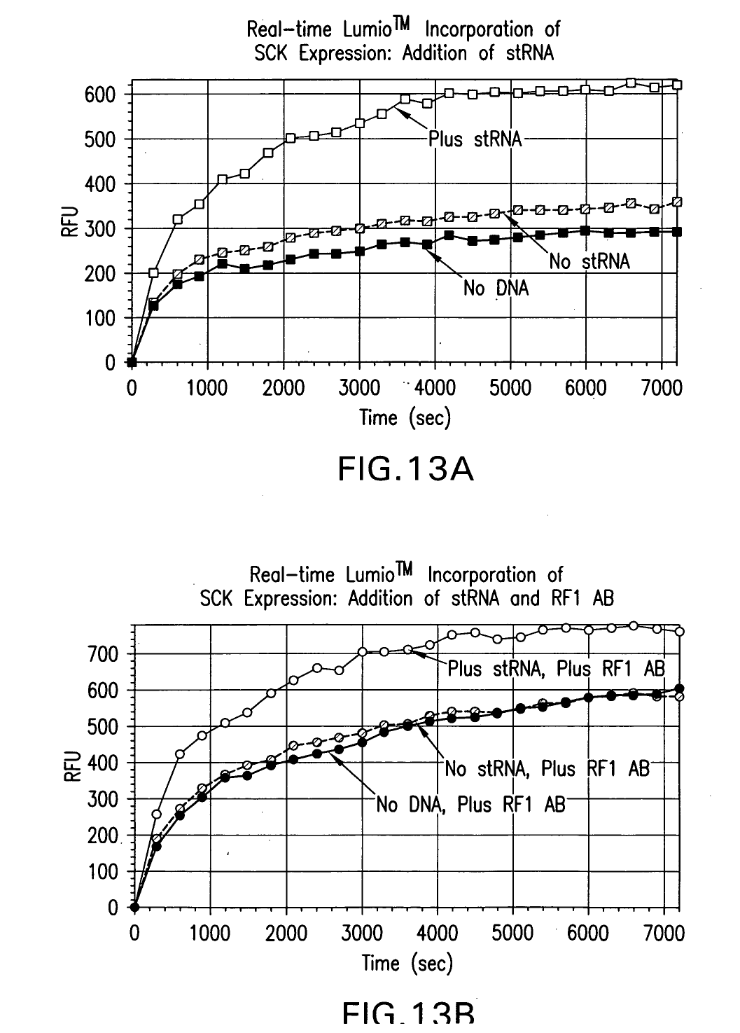

One of the advantages to of the Lumio™ Technology is the ability to monitor real-time protein expression directly in the cell-free extract. For detecting synthesis in real-time, the Lumio™ Green Detection Reagent can be added directly to the Tag-on-Demand™ expression reaction before incubation at 37° C., and Lumio™ incorporation can be observed as a Lumio™ fusion protein is being expressed. In a reaction expressing a kinase similar to creatine kinase ORF (pEXP4-SCK), the Lumio™ signal fluorescence was detected 2-fold above the pEXP4-SCK DNA control if stRNA was added to the reaction (FIG. 13A). When both the RF1 antibody and stRNA are added to the reaction, the Lumio™ signal increases but some of this increase is due to an increase in Lumio™ background signal. This increase is due to the RF1 antibody since the no DNA control reaction has the same level of signal as the pEXP4-SCK DNA control alone (FIG. 13B).

14.B. Western Blot Analysis of pEXP4 Human ORF Clones

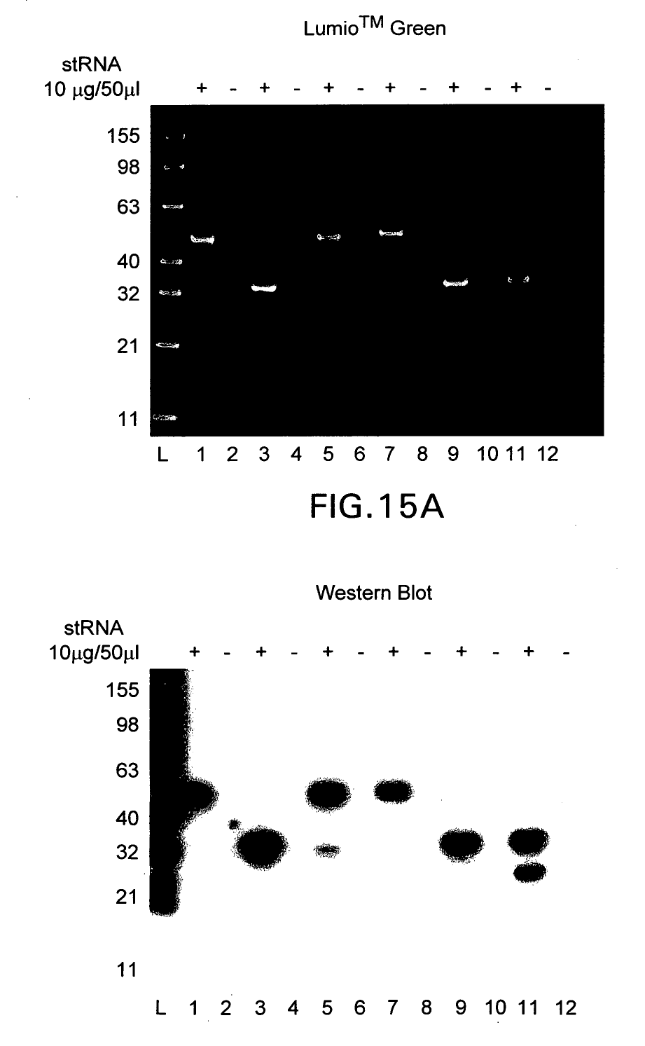

In addition to the tetracysteine t“FLASH-tag”, the pEXP4 vector also includes a 6-Histidine tag. Experiments were done to determine if detection of the His-tag is possible from the same gel as that used for in-gel detection of the tetracysteine tag. Human ORFs in the pEXP4 vector were expressed in reactions with and without the addition of suppressor T4 tRNA, loaded on a 4-12% NuPage™ Bis/Tris gel and detected using the Lumio™ Green in-gel detection kit (FIG. 15). After scanning the gel, the gel was transferred to nitrocellulose, probed with an anti-His (C-terminal)-HRP antibody. The results of the Western blot show that antibody detection of the 6-Histidine tag is possible with similar intensity to in-gel detection of the tetracysteine tag with the Lumio™ Green Reagent.

### Example 15

### Kits

An Expressway™ Lumio™ Tag-On-Demand™ Kit of the invention comprises the following components in individual containers.

IVPS E. coli S30 Extract—A19 slyD::kan strain and purified RF1 antibody (4×100 ml aliquots in 1.5 ml tubes)

2.5 ×IVPS Plus Reaction Buffer Minus tRNA

75 mM Methionine

Tag-On-Demand stRNA Mixture (10 mg/ml FPLC purified IVT expressed stRNA)

DNase/RNase-Free Distilled Water

RNase A

T7 RNA polymerase enzyme

2 ml screw-cap tubes

pEXP4-DES™ Vector

pEXP4-control plasmid (Human ORF kinase)

Lumio™ Green Detection Kit (Invitrogen)

All patents, patent publications, patent applications and other published references mentioned herein are hereby incorporated by reference in their entirety as if each had been individually and specifically incorporated by reference herein.

Examples are intended to illustrate the invention and do not by their details limit the scope of the claims of the invention. While preferred illustrative embodiments of the present invention are described, it will be apparent to one skilled in the art that various changes and modifications may be made therein without departing from the invention, and it is intended in the appended claims to cover all such deviations and modifications that fall within the true spirit and scope of the invention.

1. An in vitro protein synthesis system, comprising:
at least one extract of a cell or organism;
exogenous amino acids; and
one or more exogenous rare codon tRNAs.

2.-6. (canceled)

7. The in vitro protein synthesis system of claim 1, wherein said at least one cell extract is from prokaryotic cells.

8. The in vitro protein synthesis system of claim 7, wherein said at least one cell extract is from E. coli.

9. The in vitro protein synthesis system of claim 7, wherein said at least one rare codon tRNA is a tRNA that recognizes a codon that is present at a higher frequency in eukaryotic ORFs than in prokaryotic ORFs.

10. The in vitro protein synthesis system of claim 8, wherein said at least one rare codon tRNA is a tRNA that recognizes a codon that is present at a higher frequency in human ORFs than in E. coli ORFs.

11. The in vitro protein synthesis system of claim 10, wherein at least one of said one or more rare codon tRNAs is encoded by E. coli ileY, glyT, argX, thrU, proL or argU.

12.-13. (canceled)

14. The in vitro protein synthesis system of claim 1, further comprising at least one exogenous energy source.

15. The in vitro protein synthesis system of claim 1, further comprising at least one exogenous template nucleic acid molecule that comprises at least one open reading frame.

16. The in vitro protein synthesis system of claim 15, wherein said at least one open reading frame comprises at least one codon recognized by at least one of said one or more rare codon tRNAs.

17. (canceled)

18. The in vitro protein synthesis system of claim 1, further comprising rNTPs.

19. The in vitro protein synthesis system of claim 18, further comprising an RNA polymerase.

20. The in vitro protein synthesis system of claim 19, further comprising a DNA molecule that comprises at least one open reading frame.

21. The in vitro protein synthesis system of claim 20, wherein said at least one open reading frame comprises at least one codon recognized by at least one of said one or more rare codon tRNAs.

22. (canceled)

23. The in vitro protein synthesis system of claim 20, wherein said DNA molecule is provided in an expression vector.

24. A method of making a protein, comprising:
(a) adding to an extract of a cell or organism:
amino acids, one or more rare codon tRNAs, and
at least one ribonucleic acid template comprising at least one of the rare codons recognized by the one or more rare codon tRNAs; and
(b) incubating the extract to synthesize at least one protein encoded by the at least one ribonucleic acid template.

25.-27. (canceled)

28. A method of making a protein, comprising:
(a) adding to an extract of a cell or organism:
ribonucleotides,
an RNA polymerase,
amino acids,
one or more rare codon tRNAs, and
at least one deoxyribonucleic acid template comprising at least one of the rare codons recognized by the one or more rare codon tRNAs; and
(b) incubating the extract to synthesize at least one protein encoded by the at least one ribonucleic deoxyribonucleic acid template.

29. (canceled)

30. The method of claim 28, further comprising adding at least one exogenous energy source to said extract.

31. The method of claim 28, further comprising at least partially purifying said at least one protein.

32. A kit for in vitro protein synthesis, comprising:
an extract of a cell or organism;
amino acids; and
one or more rare codon tRNAs.

33.-35. (canceled)

36. The kit of claim 32, further comprising an expression vector.

37. The kit of claim 32, further comprising at least one exogenous energy source.

38. The kit of claim 32, wherein said extract is an E. coli S30 extract.

39.-170. (canceled)

171. The in vitro protein synthesis system of claim 1, wherein said exogenous rare codon tRNAs is a suppressor tRNA.

172. The in vitro protein synthesis system of claim 171, wherein said extract is from prokaryotic cells.

173. The in vitro protein synthesis system of claim 172, wherein said extract is from E. coli.

174. The in vitro protein synthesis system of claim 173, wherein at least one of said one or more suppressor tRNAs is a suppressor tRNA that recognizes the amber stop codon.

175. The in vitro protein synthesis composition of claim 174, wherein said suppressor tRNA that recognizes the amber stop codon is charged with serine.

176. The in vitro protein synthesis system of claim 173, further comprising at least one reagent that at least partially inhibits the activity of a release factor (RF).

177. The in vitro protein synthesis system of claim 176, wherein said at least one reagent that at least partially inhibits the activity of a release factor (RF) is at least one reagent that at least partially depletes a release factor.

178. The in vitro protein synthesis system of claim 177, wherein said at least one reagent that at least partially depletes a release factor is a specific binding partner for a release factor.

179. The in vitro protein synthesis system of claim 178, wherein said specific binding partner for a release factor is an antibody that specifically binds a release factor.

180. The in vitro protein synthesis system of claim 179, wherein at least one of said one or more suppressor tRNAs suppresses a UAA stop codon or a UAG stop codon, and said antibody specifically binds to RF1.

181. The in vitro protein synthesis system of claim 180, wherein said one or more suppressor tRNAs is a single suppressor tRNA that suppresses a UAG (amber) stop codon.

182. The in vitro protein synthesis system of claim 179, wherein said extract is an E. coli S30 extract, at least one of said one or more suppressor tRNAs suppresses a UAA stop codon or a UGA stop codon, and said antibody specifically binds to RF2.

183. The in vitro protein synthesis system of claim 179, further comprising ribonucleotide triphosphates (rNTPs).

184. The in vitro protein synthesis system of claim 183, further comprising an RNA polymerase.

185. The in vitro system of claim 184, further comprising at least one exogenous DNA template that comprises at least one open reading frame that terminates in a stop codon that is suppressed by at least one of said one or more suppressor tRNAs.

186. The in vitro protein synthesis system of claim 185, wherein said at least one exogenous nucleic acid template comprises a first open reading frame that terminates in said stop codon that is suppressed by at least one of said one or more suppressor tRNAs, and a second open reading frame contiguous with said stop codon, such that suppression of said stop codon results in translation of a fusion protein comprising said first and said second open reading frames linked by the amino acid incorporated by said at least one suppressor tRNA.


SEQUENCE LISTING (7 sequences)

SEQ ID NO: 1 - DNA, 43 bp, Artificial Sequence
  T7T4tRNA Forward Primer
ggatcctaatacgactcactataggaggcgtggcagagtggtt

SEQ ID NO: 2 - DNA, 65 bp, Artificial Sequence
  T4tRNA Reverse Primer
tggcggaggcgataggatttgaacctatgagtcgccggagcgactgccggttttagagaccggtg

SEQ ID NO: 3 - DNA, 29 bp, Artificial Sequence
  T7 forward primer used for in vitro transcription (IVT)
ggatcctaatacgactcactatatatagg

SEQ ID NO: 4 - DNA, 113 bp, Artificial Sequence
  T7 reverse primer used in vitro transcription (IVT)
tggcggaggcgataggatttgaacctatgagtcgccggagcgactgccggttttagagaccggtgcattaaaccactctgccacgcctcctatagtgagtcgtattaggatcc

SEQ ID NO: 5 - DNA, 4415 bp, Artificial Sequence
  pEXP4-DEST vector sequence
gatctcgatcccgcgaaattaatacgactcactatagggagaccacaacggtttccctctagaaataattttgtttaactttaagaaggaattatcaacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggtgatcaattcgaagcttgaagctggtggctgttgtcctggctgttgcggtggcggcaccggtcatcatcaccatcaccattgagtttgatccggctgctaacaaagcccgaaaggaagctgagttggctgctgccaccgctgagcaataactagcataaccccttggggcctctaaacgggtcttgaggggttttttgctgaaaggaggaactatatccggattaacgcttacaatttaggtggcacttttcggggaaatgtgcgcggaacccctatttgtttatttttctaaatacattcaaatatgtatccgctcatgagacaataaccctgataaatgcttcaataatgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagagtgacaccacgatgcctgtagcaatgccaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaacgcgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagacgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 6 - DNA, 3892 bp, Artificial Sequence
  pFKI005 plasmid sequence
gatctcgatcccgcgaaattaatacgactcactatagggagaccacaacggtttccctctagaaataattttgtttaactttaagaaggagatatacatatggggccttctatcgttgccaaactggaagccctgcatgaacgccatgaagaagttcaggcgttgctgggtgacgcgcaaactatcgccgaccaggaacgttttcgcgcattatcacgcgaatatgcgcagttaagtgatgtttcgcgctgttttaccgactggcaacaggttcaggaagatatcgaaaccgcacagatgatgctcgatgatcctgaaatgcgtgagatggcgcaggatgaactgcgcgaagctaaagaaaaaagcgagcaactggaacagcaattacaggttctgttactgccaaaagatcctgatgacgaacgtaacgccttcctcgaagtccgagccggaaccggcggcgacgaagcggcgctgttcgcgggcgatctgttccgtatgtacagccgttatgccgaagcccgccgctggcgggtagaaatcatgagcgccagcgagggtgaacatggtggttataaagagatcatcgccaaaattagcggtgatggtgtgtatggtcgtctgaaatttgaatccggcggtcatcgcgtgcaacgtgttcctgctacggaatcgcagggtcgtattcatacttctgcttgtaccgttgcggtaatgccagaactgcctgaggcagaactgccggacatcaacccagcagatttacgcattgatactttccgctcgtcaggggcgggtggtcagcacgttaacaccaccgattcggcaattcgtattactcacttgccgaccgggattgttgttgaatgtcaggacgaacgttcacaacataaaaacaaagctaaagcactttctgttctcggtgctcgcatccacgctgctgaaatggcaaaacgccaacaggccgaagcgtctacccgtcgtaacctgctggggagtggcgatcgcagcgaccgtaaccgtacttacaacttcccgcaggggcgcgttaccgatcaccgcatcaacctgacgctctaccgcctggatgaagtgatggaaggtaagctggatatgctgattgaaccgattatccaggaacatcaggccgaccaactggcggcgttgtccgagcaggaaggtagtgaaaacctgtacttccagtcaggtagtcatcaccatcaccatcaccattaataagcttgatccggctgctaacaaagcccgaaaggaagctgagttggctgctgccaccgctgagcaataactagcataaccccttggggcctctaaacgggtcttgaggggttttttgctgaaaggaggaactatatccggatctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcag

SEQ ID NO: 7 - protein, 6 aa, Artificial Sequence
  Sequence bound by FlAsH labeling reagent
CCXXCC